SEQUENCE LISTING (114 sequences)

SEQ ID NO: 1 - protein, 133 aa, synthetic construct
MKLSLKAGAAITAFVFAASPVLAASGADALKALNKDNDDSLEIAEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 2 - protein, 111 aa, synthetic construct
MASGADALKALNKDNDDSLEIAEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 3 - skipped

SEQ ID NO: 4 - protein, 111 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 5 - protein, 115 aa, synthetic construct
MASGADALKALNKDNDDSLEIAEVIHAGATTFTAINPDGDTTLESGETKGRLTEKDWARANKDGDQTLEMDEWLKILRTRFKRADANKDGKLTAAELDSKAGQGVLVMIMKGSGC

SEQ ID NO: 6 - protein, 115 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGSGC

SEQ ID NO: 7 - protein, 111 aa, Xanthobacter flavus
MLTGKEFLRKYNKDKDSTVEIVEAIDLGTKVFKAINPDKDKTLEAAETKGRLSDEDWAQFNKDGDKTLELDEWLIIVRKRFNDADANKDGKLTEAELDAPAGQQLILLIAK

SEQ ID NO: 8 - protein, 110 aa, synthetic construct
MGKAADAIQALDPDKDGTIDLNEAKAGAKAVFEKINPDGDGTLEVKELKGRLTKKELDAADPDNDGTLDMQEYEAVVTKQFELANPDNDGTVDEKELKTKEGKKLLKLIY

SEQ ID NO: 9 - protein, 130 aa, Methyloligella sp.
MAAILTIAGAVTVAAGGAAFAGKAADAIQALDPDKDGTIDLNEAKAGAKAVFEKINPDGDGTLEVKELKGRLTKKELDAADPDNDGTLDMQEYEAVVTKQFELANPDNDGTVDEKELKTKEGKKLLKLIY

SEQ ID NO: 10 - protein, 113 aa, Methyloligella halotolerans
MADAEISDTMKVVDPDMDNALTLEEAQAAGAKVFKKLNTDDDNTLEADELKGRVSERQLKKADPDDDGSLDMAEYEALIKKRFEAANPDGDDTIESDELETKKGKKLLELIQE

SEQ ID NO: 11 - protein, 113 aa, synthetic construct
MAPTTTTKVDIDAFDPDKDGTIDLKEALAAGSAAFDKLDPDKDGTLDAKELKGRVSEADLKKLDPDNDGTLDKKEYLAAVEAQFKAANPDNDGTIDKRELASPAGSALVNLIR

SEQ ID NO: 12 - protein, 113 aa, synthetic construct
MAPTTTTKVDIDAFDPDKDGTIDLKEALAAGSAAFDKLDPDKDGTLDAKELKGRVSEADLKKLDPDNDGTLDKKEYLAAVEAQFKAANPDNDGTIDRRELASPAGSALVNLIR

SEQ ID NO: 13 - protein, 113 aa, synthetic construct
MAPTTTTKVDIAAFDPDKDGTLDLKEALAAGSAAFDKLDPDKDGTLDAKELKGRVSEADLKKLDPDNDGTLDKKEYLAAVEAQFKAADPDNDGTLDARELASPAGSALVNLIR

SEQ ID NO: 14 - protein, 112 aa, Hyphomicrobium sp.
MGHHNCKAEMAYLNPDHDGTIDWREARRAAVRLFHKLDPDHDGTLDMKEVRGRVGILSFARFNPDRDGKLDKHEWLALVKHRFHRANPDKDGTIDCRELHSLAGRKLLRVLM

SEQ ID NO: 15 - protein, 117 aa, Hyphomicrobium sp.
MGHRSAKAHPSCPALNAIDPDGDGAMTLGEAKRAAIKTFMKLNKDGDITLELDELGGRMSAAAFAQADLIKGRGISLGEYLIEVRRRFKWANPDKDHTIECDELHSKYGRLLARLLK

SEQ ID NO: 16 - protein, 239 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGGSGGSGGSGGSGGSGGSASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 17 - protein, 251 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGGSGGSGGSGGSGGSGGSGGSGGSGGSGGSASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 18 - protein, 257 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGGSGGSGGSGGSGGSGGSGGSGGSGGSGGSGGSGGSASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 19 - protein, 263 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGGSGGSGGSGGSGGSGGSGGSGGSGGSGGSGGSGGSGGSGGSASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 20 - protein, 263 aa, synthetic construct
MASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMKGSGGSGAEAAAKEAAAKAGGSGGSAEAAAKEAAAKAGSGGSGASGADALKALNKDNDDSLEIAEVIHAGATTFTAINPDGDTTLESGETKGRLTEKDWARANKDGDQTLEMDEWLKILKTRFKRADANKDGKLTAAELDSKAGQGVLVMIMK

SEQ ID NO: 21 - protein, 113 aa, synthetic construct
MAPTTTTKVDIAAFDPDKDGTIDLKEALAAASAAFDKLDPDKDGTLDAKELKGRVSEADLKKLDPDNDGTLDKKEYLAAVEAQFKAANPDNDGTIDARELASPAGSALVNLIR

SEQ ID NO: 22 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLGAVEAQFKAANPDNDGTIDARELASPAGSALVNLIR

SEQ ID NO: 23 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAGVEAQFKAANPDNDGTIDARELASPAGSALVNLIR

SEQ ID NO: 24 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAGEAQFKAANPDNDGTIDARELASPAGSALVNLIR

SEQ ID NO: 25 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAVEGQFKAANPDNDGTIDARELASPAGSALVNLIR

SEQ ID NO: 26 - protein, 111 aa, synthetic construct
MASGADALKALNKDNDDSLEAAEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 27 - protein, 111 aa, synthetic construct
MASGADALKALNKDNDDSLEVAEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 28 - protein, 111 aa, synthetic construct
MASGADALKALNKDNDDSLEINEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 29 - protein, 111 aa, synthetic construct
MASGADALKALNKDNDDSLEISEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 30 - protein, 111 aa, synthetic construct
MASGADALKALNKDNDDSLEITEVIHAGATTFTAINPDGDTTLESGETKGRLTEKDWARANKDGDQTLEMDEWLKILRTRFKRADANKDGKLTAAELDSKAGQGVLVMIMK

SEQ ID NO: 31 - protein, 111 aa, synthetic construct
MASGADALKALNKDNDDSLEIAEVAHAGATTFTAINPDGDTTLESGETKGRLTEKDWARANKDGDQTLEMDEWLKILRTRFKRADANKDGKLTAAELDSKAGQGVLVMIMK

SEQ ID NO: 32 - protein, 111 aa, synthetic construct
MASGADALKALNKDNDDSLEIAEVVHAGATTFTAINPDGDTTLESGETKGRLTEKDWARANKDGDQTLEMDEWLKILRTRFKRADANKDGKLTAAELDSKAGQGVLVMIMK

SEQ ID NO: 33 - protein, 111 aa, synthetic construct
MASGADALKALNKDNDDSLEIAEVIHAGATTFTAINPDGDTTLESGETKGRLTEKDWARANKDGDQTLELDEWLKILRTRFKRADANKDGKLTAAELDSKAGQGVLVMIMK

SEQ ID NO: 34 - protein, 111 aa, synthetic construct
MASGADALKALNKDNDDSLEIAEVIHAGATTFTAINPDGDTTLESGETKGRLTEKDWARANKDGDQTLEADEWLKILRTRFKRADANKDGKLTAAELDSKAGQGVLVMIMK

SEQ ID NO: 35 - protein, 111 aa, synthetic construct
MASGADALKALNKDNDDSLEIAEVIHAGATTFTAINPDGDTTLESGETKGRLTEKDWARANKDGDQTLEDDEWLKILRTRFKRADANKDGKLTAAELDSKAGQGVLVMIMK

SEQ ID NO: 36 - protein, 111 aa, synthetic construct
MASGADALKALNKDNDDSLEIAEVIHAGATTFTAINPDGDTTLESGETKGRLTEKDWARANKDGDQTLEMAEWLKILRTRFKRADANKDGKLTAAELDSKAGQGVLVMIMK

SEQ ID NO: 37 - protein, 111 aa, synthetic construct
MASGADALKALNKDNDDSLEIAEVIHAGATTFTAINPDGDTTLESGETKGRLTEKDWARANKDGDQTLEMNEWLKILRTRFKRADANKDGKLTAAELDSKAGQGVLVMIMK

SEQ ID NO: 38 - protein, 111 aa, synthetic construct
MASGADALKALNKDNDDSLQIAEVIHAGATTFTAINPDGDTTLQSGETKGRLTEKDWARANKDGDQTLQMDEWLKILRTRFKRADANKDGKLTAAELDSKAGQGVLVMIMK

SEQ ID NO: 39 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAVEAQFKAADPDNDGTIDARELASPGGSALVNLIR

SEQ ID NO: 40 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAVEAQFKAADPDNDGTIDARELASPAGSGLVNLIR

SEQ ID NO: 41 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAVEGQFKAADPDNDGTIDARELASPAGSALVNLIR

SEQ ID NO: 42 - protein, 113 aa, synthetic construct
MAPTTTTKVDIAAFDPDKDGTIDLKEALAAGSAAFDKLDPDKDGTLDAKELKGRVSEADLKKLDPDNDGTLDKKEYLAAVEAQFKAADPDNDGTIDARELASPAGSALVNLIR

SEQ ID NO: 43 - protein, 18 aa, synthetic construct
GGSGGSGGSGGSGGSGGS

SEQ ID NO: 44 - protein, 111 aa, synthetic construct
  VARIANT 78: note = X is any canonical residue other than R
MASGADALKALNKDNDDSLEIAEVIHAGATTFTAINPDGDTTLESGETKGRLTEKDWARANKDGDQTLEMDEWLKILXTRFKRADANKDGKLTAAELDSKAGQGVLVMIMK

SEQ ID NO: 45 - DNA, 336 bp, synthetic construct
  source 1..336: mol_type = other DNA
atggcaagtggcgcggatgctttgaaggcgcttaacaaagacaatgacgattcgctggaaattgcagaggtaatccacgcaggcgcaactacgttcacggcaatcaacccggacggagacacaactttggagagcggagagacgaaaggacgcttgacagaaaaggattgggctagagctaataaagacggggaccagacgttggaaatggacgaatggctgaagatcctgcgtactagatttaaaagagccgatgctaataaggatggcaaattaacggctgcggagttggattccaaagcggggcaaggggtattggtcatgatcatgaaatga

SEQ ID NO: 46 - DNA, 336 bp, synthetic construct
  source 1..336: mol_type = other DNA
atggcaagtggcgcggatgctttgaaggcgcttaacaaagacaatgacgattcgctggaaattgcagaggtaatccacgcaggcgcaactacgttcacggcaatcaacccggacggagacacaactttggagagcggagagacgaaaggacgcttgacagaaaaggattgggctagagctaataaagacggggaccagacgttggaaatggacgaatggctgaagatcctgaaaactagatttaaaagagccgatgctaataaggatggcaaattaacggctgcggagttggattccaaagcggggcaaggggtattggtcatgatcatgaaatga

SEQ ID NO: 47 - DNA, 348 bp, synthetic construct
  source 1..348: mol_type = other DNA
atggcaagtggcgcggatgctttgaaggcgcttaacaaagacaatgacgattcgctggaaattgcagaggtaatccacgcaggcgcaactacgttcacggcaatcaacccggacggagacacaactttggagagcggagagacgaaaggacgcttgacagaaaaggattgggctagagctaataaagacggggaccagacgttggaaatggacgaatggctgaagatcctgcgtactagatttaaaagagccgatgctaataaggatggcaaattaacggctgcggagttggattccaaagcggggcaaggggtattggtcatgatcatgaaaggcagcggctgctga

SEQ ID NO: 48 - DNA, 348 bp, synthetic construct
  source 1..348: mol_type = other DNA
atggcaagtggcgcggatgctttgaaggcgcttaacaaagacaatgacgattcgctggaaattgcagaggtaatccacgcaggcgcaactacgttcacggcaatcaacccggacggagacacaactttggagagcggagagacgaaaggacgcttgacagaaaaggattgggctagagctaataaagacggggaccagacgttggaaatggacgaatggctgaagatcctgaaaactagatttaaaagagccgatgctaataaggatggcaaattaacggctgcggagttggattccaaagcggggcaaggggtattggtcatgatcatgaaaggcagcggctgctga

SEQ ID NO: 49 - protein, 111 aa, synthetic construct
GHHNCKAEMAYLNPDHDGTIDWREARRAAVRLFHKLDPDHDGTLDMKEVRGRVGILSFARFNPDRDGKLDKHEWLALVKHRFHRANPDKDGTIDCRELHSLAGRKLLRVLM

SEQ ID NO: 50 - DNA, 336 bp, synthetic construct
  source 1..336: mol_type = other DNA
atgctcacaggtaaagagtttctccggaaatataataaagataaggacagtaccgtcgagatcgtcgaagcgatcgatttgggtaccaaagtttttaaggcgatcaacccagataaagataagaccctcgaggctgccgagacaaagggtcgacttagtgacgaagattgggcacagttcaacaaggatggggataaaacattagaactggatgaatggctgattattgttagaaaaagatttaacgatgctgatgcaaataaggatggtaaactgacggaagcggaattggatgccccagccggacagcagctgatcctgctgatcgcgaagtga

SEQ ID NO: 51 - protein, 12 aa, synthetic construct
NKDNDDSLEIAE

SEQ ID NO: 52 - protein, 12 aa, synthetic construct
NKDKDSTVEIVE

SEQ ID NO: 53 - protein, 12 aa, synthetic construct
DPDKDGTIDLNE

SEQ ID NO: 54 - protein, 12 aa, synthetic construct
DPDMDNALTLEE

SEQ ID NO: 55 - protein, 12 aa, synthetic construct
DPDKDGTIDLKE

SEQ ID NO: 56 - protein, 12 aa, synthetic construct
DPDKDGTLDLKE

SEQ ID NO: 57 - protein, 12 aa, synthetic construct
NPDHDGTIDWRE

SEQ ID NO: 58 - protein, 12 aa, synthetic construct
DPDGDGAMTLGE

SEQ ID NO: 59 - protein, 12 aa, synthetic construct
NKDNDDSLEAAE

SEQ ID NO: 60 - protein, 12 aa, synthetic construct
NKDNDDSLEVAE

SEQ ID NO: 61 - protein, 12 aa, synthetic construct
NKDNDDSLEINE

SEQ ID NO: 62 - protein, 12 aa, synthetic construct
NKDNDDSLEISE

SEQ ID NO: 63 - protein, 12 aa, synthetic construct
NKDNDDSLEITE

SEQ ID NO: 64 - protein, 12 aa, synthetic construct
NKDNDDSLQIAE

SEQ ID NO: 65 - protein, 12 aa, synthetic construct
NPDGDTTLESGE

SEQ ID NO: 66 - protein, 12 aa, synthetic construct
NPDKDKTLEAAE

SEQ ID NO: 67 - protein, 12 aa, synthetic construct
NPDGDGTLEVKE

SEQ ID NO: 68 - protein, 12 aa, synthetic construct
NTDDDNTLEADE

SEQ ID NO: 69 - protein, 12 aa, synthetic construct
DPDKDGTLDAKE

SEQ ID NO: 70 - protein, 12 aa, synthetic construct
DPDHDGTLDMKE

SEQ ID NO: 71 - protein, 12 aa, synthetic construct
NKDGDITLELDE

SEQ ID NO: 72 - protein, 12 aa, synthetic construct
NPDGDTTLQSGE

SEQ ID NO: 73 - protein, 12 aa, synthetic construct
NKDGDQTLEMDE

SEQ ID NO: 74 - protein, 12 aa, synthetic construct
NKDGDKTLELDE

SEQ ID NO: 75 - protein, 12 aa, synthetic construct
DPDNDGTLDMQE

SEQ ID NO: 76 - protein, 12 aa, synthetic construct
DPDDDGSLDMAE

SEQ ID NO: 77 - protein, 12 aa, synthetic construct
DPDNDGTLDKKE

SEQ ID NO: 78 - protein, 12 aa, synthetic construct
NPDRDGKLDKHE

SEQ ID NO: 79 - protein, 12 aa, synthetic construct
DLIKGRGISLGE

SEQ ID NO: 80 - protein, 12 aa, synthetic construct
NKDGDQTLELDE

SEQ ID NO: 81 - protein, 12 aa, synthetic construct
NKDGDQTLEADE

SEQ ID NO: 82 - protein, 12 aa, synthetic construct
NKDGDQTLEDDE

SEQ ID NO: 83 - protein, 12 aa, synthetic construct
NKDGDQTLEMAE

SEQ ID NO: 84 - protein, 12 aa, synthetic construct
NKDGDQTLEMNE

SEQ ID NO: 85 - protein, 12 aa, synthetic construct
NKDGDQTLQMDE

SEQ ID NO: 86 - protein, 12 aa, synthetic construct
DANKDGKLTAAE

SEQ ID NO: 87 - protein, 12 aa, synthetic construct
DANKDGKLTEAE

SEQ ID NO: 88 - protein, 12 aa, synthetic construct
NPDNDGTVDEKE

SEQ ID NO: 89 - protein, 12 aa, synthetic construct
NPDGDDTIESDE

SEQ ID NO: 90 - protein, 12 aa, synthetic construct
NPDNDGTIDKRE

SEQ ID NO: 91 - protein, 12 aa, synthetic construct
DPDNDGTLDARE

SEQ ID NO: 92 - protein, 12 aa, synthetic construct
NPDKDGTIDCRE

SEQ ID NO: 93 - protein, 12 aa, synthetic construct
NPDKDHTIECDE

SEQ ID NO: 94 - protein, 12 aa, synthetic construct
DPDNDGTIDARE

SEQ ID NO: 95 - protein, 12 aa, synthetic construct
  VARIANT 2: note = X is any canonical amino acid
  VARIANT 4: note = X is any canonical amino acid
  VARIANT 7: note = X is T or S
  VARIANT 8: note = X is any hydrophobic residue
  VARIANT 10: note = X is any canonical amino acid
  VARIANT 11: note = X is any canonical amino acid
NXDXDXXXEXXE

SEQ ID NO: 96 - protein, 12 aa, synthetic construct
  VARIANT 2: note = X is any canonical amino acid
  VARIANT 4: note = X is any canonical amino acid
  VARIANT 7: note = X is T or S
  VARIANT 8: note = X is any hydrophobic residue
  VARIANT 10: note = X is any canonical amino acid
  VARIANT 11: note = X is any canonical amino acid
DXDXDGXXDXXE

SEQ ID NO: 97 - protein, 12 aa, synthetic construct
  VARIANT 2: note = X is any canonical amino acid
  VARIANT 4: note = X is G or A
  VARIANT 5: note = X is D or N
  VARIANT 6: note = X is any canonical amino acid
  VARIANT 7: note = X is T or S
  VARIANT 8: note = X is any hydrophobic residue
  VARIANT 10: note = X is any hydrophobic residue
NXDXXXXXEXDE

SEQ ID NO: 98 - protein, 12 aa, synthetic construct
NPDNDGTIDARE

SEQ ID NO: 99 - protein, 60 aa, synthetic construct
APTTTTKVDIAAFDPDKDGTIDLKEALAAGSAAFKDLDPDKDGTLDAKELKGRVSEADLK

SEQ ID NO: 100 - protein, 57 aa, synthetic construct
ASGADALKALNKDNDDSLEIAEVIHAGATTFTAINPDGDTTLESGETKGRLTEKDWA

SEQ ID NO: 101 - protein, 52 aa, synthetic construct
KLDPDNDGTLDKKEYLAAVEAQFKAANPDNDGTIDARELASPAGSALVNLIR

SEQ ID NO: 102 - protein, 53 aa, synthetic construct
RANKDGDQTLEMDEWLKILRTRFKRADANKDGKLTAAELDSKAGQGVLVMIMK

SEQ ID NO: 103 - protein, 12 aa, synthetic construct
NPDGDTTLESGE

SEQ ID NO: 104 - protein, 15 aa, synthetic construct
DPDKDGTIDLKEALA

SEQ ID NO: 105 - protein, 12 aa, synthetic construct
DPDKDGTLDAKE

SEQ ID NO: 106 - protein, 15 aa, synthetic construct
DKDGDGTITTKEIII

SEQ ID NO: 107 - protein, 12 aa, synthetic construct
DADGNGTIDFPE

SEQ ID NO: 108 - protein, 18 aa, synthetic construct
NKDGDQTLEMDEWLKILR

SEQ ID NO: 109 - protein, 12 aa, synthetic construct
DANKDGKLTAAE

SEQ ID NO: 110 - protein, 18 aa, synthetic construct
DPDNDGTLDKKEYLAAVE

SEQ ID NO: 111 - protein, 12 aa, synthetic construct
NPDNDGTIDARE

SEQ ID NO: 112 - protein, 12 aa, synthetic construct
DKDGNGFISAAE

SEQ ID NO: 113 - protein, 12 aa, synthetic construct
DIDGDGQINYES

SEQ ID NO: 114 - protein, 15 aa, synthetic construct
NKDNDDSLEIAEVIH

(19) United States
(12) Patent Application Publication
(10) Pub. No.: US 2026/0098064 A1
(43) Pub. Date: Apr. 9, 2026

(54) LANMODULIN ORTHOLOGS WITH IMPROVED RARE EARTH SEPARATION PERFORMANCE

(71) Applicants: The Penn State Research Foundation, University Park, PA (US); Lawrence Livermore National Security, LLC, Livermore, CA (US)

(72) Inventors: Joseph Alfred COTRUVO, Jr., University Park, PA (US); Joseph Anthony MATTOCKS, University Park, PA (US); Amie Kathleen BOAL, University Park, PA (US); Jonathan JUNG, University Park, PA (US); Chi-Yun LIN, University Park, PA (US); Ziye DONG, Livermore, CA (US); Dan M. PARK, Livermore, CA (US); Wonseok CHOI, State College, PA (US)

(21) Appl. No.: 19/112,328

(22) PCT Filed: Sep. 18, 2023
(86) PCT No.: PCT/US2023/074516

§ 371 Date: Mar. 17, 2025

Related U.S. Application Data
(60) Provisional application No. 63/376,060, filed on Sep. 16, 2022.
(60) Provisional application No. 63/505,052, filed on May 30, 2023.

(51) Int. Cl. C07K 14/195; G01N 1/40; G01N 33/18

(52) U.S. Cl. CPC C07K 14/195; G01N 1/4077; G01N 33/1813

(57) ABSTRACT

Provided are proteins that bind rare earth metals. The proteins may have enhanced REE/REE selectivity. The proteins may have four EF hand motifs each having 11, 12, or 13 amino acids residues. Each EF hand motif is separated by 12 or 13 amino acid residues, where each amino acid residue is any canonical amino acid residue and at least one amino acid residue is a hydrophobic amino acid residue. When the EF hand motif has 12 amino acid residues, the motif may have the following sequence: X¹-X²-X³-X⁴-X⁵-X⁶-X⁷-X⁸-X⁹-X¹⁰-X¹¹-E. Also provided are devices and kits comprising a protein of the present disclosure. Also provided are methods of using the proteins and devices.

### CROSS REFERENCE TO RELATED APPLICATIONS

This application claims priority to U.S. Provisional Application No. 63/376,060, filed Sep. 16, 2022 and U.S. Provisional Application No. 63/505,052, filed May 30, 2023, the disclosures of which are incorporated herein by reference.

### STATEMENT REGARDING FEDERALLY SPONSORED RESEARCH

This invention was made with government support under Grant Nos. DE-SC0021007 and DE-AC52-07NA27344 awarded by the Department of Energy, Grant No. CHE-1945015 awarded by National Science Foundation, and Grant No. GM119707 awarded by National Institutes of Health. The Government has certain rights in the invention.

### SEQUENCE LISTING

The instant application contains a Sequence Listing, which has been submitted in .xml format and is hereby incorporated by reference in its entirety. Said .xml copy was created on Sep. 18, 2023, is named “074339_00251_ST26.xml”, and is 114,140 bytes in size.

### BACKGROUND OF THE DISCLOSURE

The irreplaceable roles of rare earth (RE) elements in ubiquitous modern technologies ranging from permanent magnets to LEDs and phosphors have renewed interest in one of the grand challenges of separation science—efficient separation of lanthanides. The separation of these 15 elements is complicated by the similar physicochemical properties of their predominating +III ions, with ionic radii decreasing only 0.19 Å between La^{III} and Lu^{III}, which also leads to these metals co-occurring in RE-bearing minerals. Conventional hydrometallurgical liquid-liquid extraction methods for RE production utilize organic solvents like kerosene and toxic phosphonate extractants and require dozens or even hundreds of stages to achieve high-purity individual RE oxides. The inefficiency and large environmental impact of RE separations have stimulated research efforts into alternative ligands with larger separation factors between adjacent REs, and greener process designs to achieve RE separation in fewer stages and using all-aqueous chemistry.

The discovery of the founding member of the lanmodulin (LanM) family of lanthanide-binding proteins demonstrated that Nature has evolved macromolecules surpassing the selectivity of synthetic f-element chelators. The prototypal LanM, from Methylorubrum extorquens AM1 (Mex-LanM), is a small (12-kDa), monomeric protein that undergoes a selective conformational response to picomolar concentrations of lanthanides and actinides, has facilitated understanding of lanthanide uptake in methylotrophs, and has served as a technology platform for f-element detection, recovery, and separation. Unusually among RE chelators, Mex-LanM favors the larger and more abundant light REs (LREs), especially La^{III}-Sm^{III}, over heavy REs (HREs).

### SUMMARY OF THE DISCLOSURE

The present disclosure provides proteins that bind rare earth metals. Also provided are devices and kits comprising a protein of the present disclosure. Also provided are methods of using the proteins and devices.

In an aspect, the present disclosure provides proteins that bind metals (e.g., lanthanides and/or actinides). Other metal-binding proteins are disclosed in WO2020051274 and WO2023004333, which are incorporated herein by reference.

A protein of the present disclosure may be of various lengths. For example, a protein of the present disclosure has 65 to 160 amino acid residues, including all integer amino acid values and ranges therebetween. For example, the protein has a molecular weight of around 8 kDa to 14 kDa, including all 0.1 Da values and ranges therebetween (e.g., ˜12 kDa). A protein of the present disclosure comprises at least one segment where one or more rare earth metals can bind. In various examples, the segment has at least 70% homology (e.g., at least 70%, at least 75%, at least 80%, at least 85%, at least 90%, or at least 95% homology) with the sequence of Hansschlegelia quercus LanM, which may be referred to as Hans-LanM. In various other examples, the protein is truncated. For example, the protein is truncated at the N-terminus via deletion of the first 10, 20, 30, or 40 residues of the full translated sequence. In another example, the protein is truncated at the C-terminus via deletion of the last 10, 20, 30, or 40 residues of the full translated sequence. In various examples of truncated sequences, EF hands 2 and 3 remain, as well as the majority of the hydrophobic core of the protein.

In various examples, a protein or peptide of the present disclosure capable of dimerization upon contact with a metal/metal ion may comprise 4 EF hand motifs (e.g., a first EF hand motif, a second EF hand motif, a third EF hand motif, and a fourth EF hand motif), each EF hand motif comprising 11, 12, or 13 amino acid residues (e.g., 12 amino acid residues). Each EF hand motif is separated by 12 or 13 amino acid residues, where each amino acid residue is any canonical amino acid residue and at least one amino acid residue is a hydrophobic amino acid residue. In the case of the third EF hand motif (i.e., EF3) and the fourth EF hand motif (i.e., EF4), they are separated by the sequence (X)₅-R-(X)₆, where each X is any canonical amino acid residue. When the EF hand motif has 12 amino acid residues, the motif may have the following sequence:

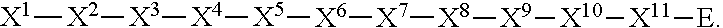

For the first EF hand motif (i.e., EF1), the second EF hand motif (i.e., EF2), and EF4, X¹ is D or N; X³ is D, N, or E; X⁵ is D, N, or E; X⁸ is a hydrophobic residue; X⁹ is D, E, or T; X¹⁰ is a hydrophobic residue; and X², X⁴, X⁶, X⁷, and X¹¹ are each independently any canonical amino acid residue. In various examples, X⁷ of EF1, EF2, and/or EF4 is T or S. For EF3, X¹ is N; X³ is D; X⁴ is G or A; X⁵ is D or N; X⁷ is T or S; X⁸ is a hydrophobic residue, X⁹ is E; X¹⁰ is a hydrophobic residue; X¹¹ is D; and X² and X⁶ are each independently any canonical amino acid residue (e.g., N-X²-D-X⁴-X⁵-X⁶-X⁷-X⁸-E-X¹⁰-D-E (SEQ ID NO:97). In various examples, X⁴ of EF3 is A. In various examples, X⁸ of EF3 is L. In various examples, X¹⁰ of EF3 is L, I, or M. Without intending to be bound by any particular theory, it is considered that the dimerization strength of such a protein depends on the identity of the metal ion bound, where dimers form preferentially in the presence of a trivalent rare earth element or actinide. A protein having this sequence may be connected to another protein of the present disclosure via a peptide linker as described herein.

In various examples, a protein or peptide of the present disclosure may have enhanced REE/REE selectivity. The selectivity may be between light rare earth metals and heavy rare earth metals, and more than that of M. extorquens lanmodulin. Such a protein may comprise 4 EF hand motifs (e.g., a first EF hand motif, a second EF hand motif, a third EF hand motif, and a fourth EF hand motif), each EF hand motif comprising 11, 12, or 13 amino acid residues and each EF hand motif is separated by 12 or 13 amino acid residues, where each residue is a canonical residue and at least one amino acid residue is a hydrophobic amino acid residue. When the EF hand motif has 12 amino acid residues, the motif may have the following sequence:

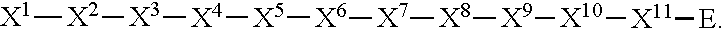

For the first EF hand motif (i.e., EF1) and fourth EF hand motif (i.e., EF4), X¹ is D or N; X³ is D, N, or E; X⁵ is D, N, or E; X⁸ is a hydrophobic residue; X⁹ is D, E, or T; X¹⁰ is a hydrophobic residue; and X², X⁴, X⁶, X⁷, and X¹¹ each independently any canonical amino acid residue. In various examples, X⁷ of EF1 and/or EF4 is T or S. For EF2, X¹ is N; X³ is D; X⁵ is D; X⁷ is T or S; X⁸ is a hydrophobic residue; X⁹ is E; X¹² is E; and X², X⁴, X⁶, X¹⁰, and X¹¹ are each independently any canonical amino acid residue (e.g., N-X²-D-X⁴-D-X⁶-X⁷-X⁸-E-X¹⁰-X¹¹-E (SEQ ID NO:95). In various examples, X⁸ of EF2 is L, I M, or V. For EF3, X¹ is D; X³ is D; X⁵ is D; X⁶ is G; X⁷ is T or S; X⁸ is a hydrophobic residue; X⁹ is D; and X², X⁴, X¹⁰, and X¹¹ are each independently any canonical residue (e.g., D-X²-D-X⁴-D-G-X⁷-X⁸-D-X¹⁰-X¹¹-E (SEQ ID NO:96). In various embodiments, X⁸ of EF3 is L, I, M, or V. At least one X² of either EF2 or EF3 is P. A protein having this sequence may be connected to another protein of the present disclosure via a peptide linker as described herein.

In an aspect, the present disclosure provides devices. The device comprises one or more proteins of the present disclosures.

In an aspect, the present disclosure provides kits. The kits may provide one or more proteins of the present disclosure and/or one or more devices of the present disclosure. The kit may include instructions for use of the proteins or devices.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

A method of the present disclosure may be a method of detecting and/or quantifying the amount of one or more lanthanides and/or actinides in a sample. The method may comprise contacting the sample with one or more proteins and/or device of the present disclosure. The contacted sample may then be exposed to light and the resulting emission of the exposed contacted sample. The resulting emission results may then be compared to a known standard curve for a specific lanthanide or actinide. The concentration may then be determined by that comparison. Known standard curves may be prepared based on the desire to detect and/or determine the quantity of any specific lanthanide or actinide. Methods of preparing standard curves are known in the art.

A method of using a protein and/or device of the present disclosure may be a method for binding one or more rare earth metals (e.g., lanthanides and/or actinides) in a sample. Binding may occur by contacting the sample with one or more proteins and/or devices of the present disclosure. The method may be performed on various types of samples. Examples of samples include, but are not limited to drinking water, wastewater, ground water, ash ponds, aqueous extract from contaminated soil, drainage (e.g., mine drainage, such as, for example, acidic mine drainage) or leachate (e.g., electronic waste leachate or leachate of an ore leachate). In various other examples, the sample is a solid sample. The method may be applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

Various lanthanides (e.g., lanthanide ions) and/or actinides (e.g., actinide ions) may be bound by a protein and/or device. For example, the lanthanide is chosen from Tb, Eu, Dy, Sm, Nd, and ions thereof. In various examples, the lanthanide is Tb or an ion thereof. The bound lanthanides and/or actinides may be the same or different. The concentration of the lanthanide and/or actinide in the sample may be less than 1 ppm.

### BRIEF DESCRIPTION OF THE FIGURES

For a fuller understanding of the nature and objects of the disclosure, reference should be made to the following Examples taken in conjunction with the accompanying figures therein.

FIG. 1 shows Hans-LanM diverges from Mex-LanM in sequence and RE/RE selectivity. A, Sequence similarity network (SSN) of core LanM sequences indicate Hans-LanM forms a distinct cluster. The SSN includes 696 LanM sequences connected with 48,647 edges, thresholded at a BLAST E-value of 1×10⁻⁵ and 65% sequence identity. The black box encloses nodes clustered with Hans-LanM. The LanM sequence associated with Mex (▾) and four within Hansschlegelia (▴) are enlarged compared to other nodes (o). Colors of the nodes represent the family from which the sequences originate. B, Comparison of the sequences of the 4 EF-hands of Mex- and Hans-LanMs. C, Circular dichroism spectra from a representative titration of Hans-LanM with La^{III}, showing the metal-associated conformational response increasing helicity; apoprotein is bold black, La^{III}-saturated protein is bold red. D, CD titration of Hans-LanM with La^{III}, Nd^{III}, and Dy^{III} (pH 5.0). Each point represents the mean±s.d. from three independent experiments. e, Comparison of K_{d,app} values (pH 5.0) for Mex-LanM and Hans-LanM, plotted versus ionic radius. Mean±s.e.m. from three independent experiments.

FIG. 2 shows a dimerization equilibrium sensitive to LRE vs. HRE or non-RE coordination. a, Apparent molecular weight of Hans-LanM complexes with REs as determined by analytical SEC (lines) or SEC-MALS (dashes). See Table 1 for conditions. Each individual data point is the result of a single experiment. b, The La^{III}-bound Hans-LanM dimer as determined by X-ray crystallography. La^{III} ions are spheres and Na^{I} ions are gray spheres. c, Detailed view of the dimer interface near EF3 of chain A (cartoon). Arg100 from chain C (cartoon) anchors a hydrogen-bonding network involving Asp93 of chain A and two EF3 La^{III} ligands (Glu91 and Asp85). These interactions constitute the sole polar contacts at the dimer interface, providing a means to control the radius of the lanthanide-binding site at EF3. d, Schematic of the interactions at the dimer interface. Dashed lines indicate hydrogen-bonding interactions and other dashed lines indicate hydrophobic contacts. e, DENSS projections of electron density from SAXS datasets for La^{III}-bound (left) and Dy^{III}-bound (right) Hans-LanM, overlaid with a PyMOL-generated ribbon diagram of the dimeric La^{III}-Hans-LanM crystal structure.

FIG. 3 shows Hans-LanM uses an extended hydrogen-bonding network to control lanthanide selectivity. a, Zoomed-in views of EF2 and EF3 in La^{III}-Hans-LanM. La^{III} ions are shown as green spheres. Coordination bonds and hydrogen-bonds are shown as dashed lines. Residues contributed by chain A are shown and those contributed by chain C (in the case of EF3) are also shown. (Inset) Overlay of La^{III}-Hans-LanM with Dy^{III}-Hans-LanM, showing Glu91's carboxylate shift. b, Representative metal-binding site (EF3) in Nd^{III}-Mex-LanM. Nd^{III} ion shown as sphere. Solvent molecules are shown as spheres.

FIG. 4 shows leveraging Hans-LanM to separate Nd/Dy in a single-stage process. a, Hans-LanM and the R100K variant display greater differences in Nd vs. Dy complex stability than Mex-LanM against desorption by citrate. Mean s.e.m. for three independent trials. **Significant difference between [citrate]_{1/2} for La^{III} between Hans-LanM and R100K-Hans-LanM (20 μM protein) shows the impact of dimerization of La^{III} complex stability (p<0.01, ANOVA with Bonferroni post-test). Mex-LanM Nd and Dy data from Dong et al., ACS Cent. Sci. 7, 1798, 2021. b, Spectrofluorometric titration of Hans-LanM and R100K variant (λₑₓ=280 nm, λₑₘ=333 nm) at pH 5.0, depicting the malonate-induced desorption of a 2:1 metal:protein complex. Mean±s.e.m. for three independent trials, except R100K, which were single trials of each condition. c, Comparison of distribution factors (pH 5.0, ˜0.33 mM each RE, La^{III}-Dy^{III}) for immobilized Hans-LanM, R100K-Hans-LanM, and Mex-LanM. Each point represents mean±s.d. for three independent trials. d, Separation of a 95:5 mixture of Nd:Dy using immobilized R100K-Hans-LanM and a desorption scheme of three stepped concentrations of malonate followed by pH 1.5 HCl. One bed volume was 0.7 mL.

FIG. 5 shows sequence alignment of Mex-LanM and Hans-LanM (after signal peptide removal), showing 33% sequence identity. EF-hands are shown in bold. See Table 12 for the full-length Hans-LanM sequence including the predicted signal peptide. The Hans-LanM protein used in the present study consisted of residues A24-K133. The sequences shown are

(SEQ ID NO: 99)
APTTTTKVDIAAFDPDKDGTIDLKEALAAGSAAFKDLDPDKDGTLDAKE
LKGRVSEADLK,
(SEQ ID NO: 100)
ASGADALKALNKDNDDSLEIAEVIHAGATTFTAINPDGDTTLESGETKG
RLTEKDWA,
(SEQ ID NO: 101)
KLDPDNDGTLDKKEYLAAVEAQFKAANPDNDGTIDARELASPAGSALVN
LIR,
and
(SEQ ID NO: 102)
RANKDGDQTLEMDEWLKILRTRFKRADANKDGKLTAAELDSKAGQGVLV
MIMK.

FIG. 6 shows stoichiometric metal titrations of Hans-LanM (15 μM) with La^{III} and Dy^{III}, monitored by CD spectroscopy. Molar ellipticity at 222 nm is plotted against equivalents of metal added. Experimental conditions: 20 mM acetate, 100 mM KCl, pH 5.0. Each data point is the mean±SD for two independent measurements. Taken together with FIG. 1, these observations suggest the following model for Hans-LanM's interactions with RE^{III} ions: tight (though preferential for LREs) metal binding to one site results in a conformational change; a second site responds to LREs cooperatively with the first to give the complete conformational response, but it responds to Dy non-cooperatively and only at >0.5 μM concentrations; and a third site does not cause an observable conformational change with any RE. It is posulated that the third site is EF1 based on prior work with Mex-LanM and the crystal structures described herein; EF2 and EF3 cannot definitively distinguished as the first and second sites. This more complex response profile than that of Mex-LanM appears tuned to ensure a full, cooperative response only to LREs.

FIG. 7 shows size-exclusion chromatograms of apo Hans-LanM (black) and Hans-LanM metalated with 3.0 equiv. La^{III} (red). The S75 column volume was 24 mL. The apoprotein, like apo-Mex LanM, elutes over a wide molecular weight range (30-70 kDa), suggestive of multiple, disordered conformations. The La^{III}-bound protein displays soluble higher molecular weight species that form when REs are added to Hans-LanM at high concentrations, as well as a symmetrical peak at ˜28 kDa suggestive of a dimer.

FIG. 8 shows size-exclusion chromatography of RE^{III}-Hans-LanM complexes (RE=La, Nd, Sm, Eu, Gd, Tb, Dy, Ho, Er, Tm, Y). Apo-Hans (590 μM, 100 μL) was metalated with 3.0 equiv. RE, 0.5 equiv. at a time with mixing, and loaded to a 24 mL analytical S75 column calibrated as described. a, Chromatograms for RE=La−Dy show an apparent molecular weight suggestive of a Hans-LanM dimer for La and Nd, which progressively shifts to lower apparent molecular weight, likely indicating decreasing proportion of dimer in rapid equilibrium with monomer. b, Beyond Dy, the speciation of the complexes is complicated but suggests emergence of a more extended monomer or a slower-exchanging dimer population.

FIG. 9 shows SEC-MALS traces for La-, Nd-, and Dy-bound Hans-LanM, illustrating the later elution and lower weight-average molar mass of the Dy complex (see Table 3). Apo-Hans-LanM was incubated with 3 equiv. each RE^{III} ion, precipitate and aggregate were removed by centrifugation and analytical SEC, and the protein was injected to the column at a concentration of 114-128 μM.

FIG. 10 shows dimer dissociation of apo Hans-LanM followed by ITC. (Top) Representative ITC trace for titration of 300 μM protein into buffer. (Bottom) Thermogram derived from the data above fitted to the dimer dissociation model using the NanoAnalyze software, with parameters presented in Table 18. Conditions: 30 mM MOPS, 100 mM KCl, pH 7.0, 30° C.

FIG. 11 shows dimer dissociation of Dy^{III}₂-Hans-LanM followed by ITC. (Top) Representative ITC trace for titration of 300 μM protein into buffer. (Bottom) Thermogram derived from the data above fitted to the dimer dissociation model using the NanoAnalyze software, with parameters presented in Table 18. Conditions: 30 mM MOPS, 100 mM KCl, pH 7.0, 30° C. Utilizing Eq. 1, the concentrations of Dy^{III}-Hans-LanM monomer and dimer under the conditions of SEC-MALS can be calculated, given [P]=18.9 μM (obtained from Table 17) and K_{dimer}=60 μM obtained from ITC (Table 18). We obtain [M]=13.5 μM and thus [D]=3.3 μM. Because [D] is ˜25% of [M], this result corresponds well to the average mass of 15.5 kDa obtained by SEC-MALS for this form, which is ˜25% higher than the expected MW of 11.9 kDa, suggesting partial dimerization. This, in turn, supports our interpretation that the SEC-MALS results indicate rapid monomer-dimer equilibria where the individual monomer and dimer components cannot be resolved, therefore manifesting as a weighted average of the two populations.

FIG. 12 shows titration of La^{III}₂-Hans LanM followed by ITC. (a, Top) Representative ITC trace for titration of 150 μM protein into buffer. (a, Bottom) Thermogram derived from the data above. (b, Top) Representative ITC trace for titration of 540 μM protein into buffer with an initial 0.2 μL injection followed by 9×5.0 μL additions. (b, Bottom) Thermogram derived from the data above. The inability to observe differences between the heats of each injection in these experiments reinforces the very tight binding in the La^{III}-bound dimer. Conditions: 30 mM MOPS, 100 mM KCl, pH 7.0, 30° C. We can estimate the maximum dimer dissociation constant for La^{III}-Hans-LanM by using Eq. 1, the peak concentration from SEC-MALS, [P]=18.4 μM (Table 17), and conservatively assuming that the minimum threshold for observable monomer [M] is 10% of the total protein concentration (1.84 μM), because it would appear in the SEC-MALS as a roughly 1 kDa difference from the theoretical monomer MW of 11.9 kDa (such a difference is observable in the case of apo-Hans-LanM, see Table 17). In this case, solving for K_{dimer} yields 0.4 μM, which represents the maximum dissociation constant for the dimer of the La^{III}-bound protein. Therefore, Hans-LanM exhibits a more than 100-fold enhanced dimerization in the presence of La^{III} versus Dy^{III}.

FIG. 13 shows a comparison of topology and key residues in Hans- and Mex-LanMs. a, Topology diagrams for La^{III}-Hans-LanM and Nd^{III}-Mex-LanM. Despite possessing only 33% sequence identity, the overall topologies of the two proteins are very similar, with three core helices (α1-3) forming the central three-helix bundle, and decorated with two auxiliary helices, preceding EF1 and EF3. As explained in FIG. 32, the presence of a Nd^{III} ion in EF4 of Mex-LanM is a result of the high protein concentration used for crystallization. b, Sequence alignment of key regions of Hans- and Mex-LanMs and H. sapiens calmodulin. The similarity of metal-binding residues in these proteins is highlighted in light blue; note the presence of Glu residues at the 9^{th} position of each EF-hand in Hans-LanM, uniquely relative to the other proteins. The residues that are involved in interactions at the dimer interface in Hans-LanM are bolded (hydrogen bonding interactions: E91, D93, R100) (hydrophobic interactions: I43, I47, H48, T63, M92, L96). The charges of residues corresponding to Hans-LanM D93 and R100 are reversed (K94, E101) in Mex-LanM. Several of the residues involved in hydrophobic interactions (I43, I47, L96) in Hans-LanM correspond to similar residues in Mex-LanM (L44, L48, L97), whereas the others (H48, T63, M92) correspond to residues that either lack bulky sidechains (A49, G64) or are charged (K94). This analysis also highlights another potentially important difference, that calmodulin possesses highly conserved Gly residues at both the 4^{th} and 6^{th} positions of each EF-hand; Gly residues are conserved at those positions in 81-100% of predicted calmodulin sequences, depending on the EF-hand/residue position. By contrast, Hans- and Mex-LanM EF-hands only have a glycine at one of those positions (or none, in Hans-LanM EF1). Sequences shown are NKDNDDSLEIAEVIH (SEQ ID NO:114), NPDGDTTLESGE (SEQ ID NO:103), DPDKDGTIDLKEALA (SEQ ID NO:104), DPDKDGTLDAKE (SEQ ID NO: 105), DKDGDGTITTKEIII (SEQ ID NO:106), DADGNGTIDFPE (SEQ ID NO: 107), NKDGDQTLEMDEWLKILR (SEQ ID NO:108), DANKDGKLTAAE (SEQ ID NO:109), DPDNDGTLDKKEYLAAVE (SEQ ID NO:110), NPDNDGTIDARE (SEQ ID NO:111), DKDGNGFISAAE (SEQ ID NO:112), and DIDGDGQINYES (SEQ ID NO:113)

FIG. 14 shows SAXS raw data for La-, Nd-, and Dy-bound Hans-LanM. SAXS datasets were collected on SEC-MALS fractions in presence of different metal ions in 30 mM MOPS, 100 mM KCl, 5% glycerol, pH 7.0, on an in-house Rigaku BioSAXS2000^{nano}. a, The La-bound protein was 1.5 mg/mL, the Nd-bound protein was 1.4 mg/mL, and the Dy-bound protein was 1.6 mg/mL. SAXS data sets were collected for 60 min with six 10-min images using the autosampler quartz flow cell. Buffer SAXS data were collected over 60 min using the same flow cell and were used for the reference subtraction. The overlays of the six 10-min images that were averaged in each case showed that there was no X-ray radiation damage. b, and c, CRYSOL (ATSAS) fits of the La and Dy SAXS data to the crystallographic La-Hans-LanM and Dy-Hans-LanM dimer models. Chi-squared values are listed in Table 6.

FIG. 15 shows overlay of the Guinier plots for La-, Nd-, and Dy-bound Hans-LanM. The fits point to radii of gyration (R_{g}) of 18.5±2.5 Å (La), 18.7±2.7 Å (Nd), and 17.8±2.6 Å (Dy) (see Table 4). Note that there is less difference between the R_{g} values for the Dy and the La/Nd complexes than might be expected based on SEC-MALS (Table 17 suggests a ˜2-3 Å difference in hydrodynamic radius for La/Nd vs. Dy), because the protein concentrations used for SAXS are 5-fold higher than those for SEC-MALS, and therefore the population of Dy-bound dimer is substantially larger in the SAXS experiment. Nevertheless, these RE-dependent differences are within the uncertainty of the SAXS R_{g} values.

FIG. 16 shows La^{III}-bound Hans-LanM solvent envelope. DENsity from Solution Scattering (DENSS) is an algorithm for calculating ab initio electron density maps from solution scattering data. The DENSS electron density map, shown as a transparent surface in the PyMOL-generated representation, overlays with the dimeric La^{III}-Hans-LanM crystal structure, colored here by chain (light blue and green). La^{III} ions are represented as gold spheres. The color ramp is from lowest to highest electron densities: blue (2σ) to cyan (5σ) to green (7.5σ) to yellow (10σ) to red (15σ). Manual fitting of the envelope and model was performed in PyMOL. The CRYSOL fit of the calculated SAXS profile overlays with the experimental SAXS profile with a Chi-squared fit of 1.1 (Table 6). The left panel is identical to the La^{III}-Hans-LanM data shown in FIG. 2e.

FIG. 17 shows Nd^{III}-bound Hans-LanM solvent envelope. The DENSS electron density map, shown as a transparent surface in the PyMOL-generated representation, overlays with the dimeric La^{III}-Hans-LanM crystal structure, colored here by chain (light blue and green) with La^{III} ions represented as gold spheres. Manual fitting of the envelope and model was performed in PyMOL. The CRYSOL fit of the calculated SAXS profile overlays with the experimental SAXS profile with a Chi-squared fit of 1.3 (Table 6).

FIG. 18 shows Dy^{III}-bound Hans-LanM solvent envelope. The DENSS electron density map, shown as a transparent surface in the PyMOL-generated representation, overlays poorly with the dimeric La^{III}-Hans-LanM crystal structure. The CRYSOL fit of the calculated SAXS profile for the dimer overlays with the experimental SAXS profile with a Chi-squared fit of 3.8 (Table 6).

FIG. 19 shows distance distribution (P(r)) analysis, which suggests that Hans-LanM undergoes a transition from a single species with La and Nd to two states with Dy. Overlay of the pair-wise distance distribution functions, P(r), for the La- (green), Nd- (blue), and Dy-bound (red) Hans-LanM datasets. The R_{g} values from this analysis—18.5 Å (La), 18.7 Å (Nd), and 17.6 Å (Dy)—are similar to the R_{g} values obtained from the Guinier analysis. For La and Nd complexes, the P(r) has a bell shape, representative of scattering from a globular particle. In the case of Dy, P(r) shows three shoulders suggestive of a mixture of species (e.g., monomer/dimer mixture).

FIG. 20 shows Kratky plots suggest that Hans-LanM flexibility increases from La to Nd to Dy. Kratky plots for La- (green), Nd- (blue), and Dy-bound (red) Hans-LanM. Kratky plots derived from the SAXS data qualitatively inform on a protein's flexibility and/or foldedness. The progressive divergence from the q axis from La- to Nd- to Dy-bound forms is suggestive of increasing disorder, which may relate to weaker Dy^{III} binding and reduced cooperativity (FIG. 1d) and/or to monomer-dimer equilibrium. Units of q are Å⁻¹.

FIG. 21 shows a 2Fₒ-F_{c} electron density map (gray mesh, contoured at 1.0 σ) and anomalous difference map (purple mesh, contoured at 3.0 σ) of EF-hands 3 and 4 of La^{III}-bound Hans-LanM. a, In EF4, solvent is clearly indicated, and the lack of anomalous difference density shows that La^{III} does not occupy this site. The metal ion was modeled as a fully occupied Na^{I} for reasons described in FIG. 52. b, In EF3, representative of EF-hands 1-3, the electron density map suggests no solvent coordination, and the anomalous difference density map is consistent with a fully occupied La^{III} ion.

FIG. 22 shows comparison of the structures of LanM EF-hands with an EF-hand from calmodulin (CaM) and with a lanthanide-dependent methanol dehydrogenase (MDH). a, EF2 from La^{III}-bound Hans-LanM. The La^{III} ion is a green sphere. b, EF3 from Nd^{III}-bound Mex-LanM. The Nd^{III} ion is an aqua sphere and coordinated solvent molecules (w1 and w2) are red spheres. c, EF2 from H. sapiens CaM (PDB code: 1CLL). The Ca^{II} ion is a gray sphere and a coordinated solvent molecule is a red sphere. d, The active site of the lanthanide-dependent MDH, XoxF, from Methylomicrobium buryatense 5GB1C, metalated with La^{III} (1.85 Å resolution, PDB code: 6DAM). The 10-coordinate La^{III} ion is shown as a green sphere. The enzyme also requires a pyrroloquinoline quinone cofactor (PQQ). The primary coordination spheres of Nd^{III}-Mex-LanM and Ca^{II}-CaM are nearly identical except the D5 residue is monodentate in CaM (Asp24) but bidentate in Mex-LanM (Asp88) and there is an extra water molecule (w1) in Mex-LanM. These images also highlight the consequence of Hans-LanM having an Asn at the 1^{st} position of the EF-hands (N₁) versus an Asp (D₁) in Mex-LanM and CaM. This substitution induces a different peptide backbone structure between the 4^{th} and 6^{th} EF-hand positions. In Hans-LanM, the non-coordinated sidechain Nδ of Asn58 hydrogen bonds with the backbone CO of Asp62 residue (5^{th} position), whereas in Mex-LanM and CaM the non-coordinated atom is an oxygen, enabling hydrogen bonding with the backbone NH of the 6^{th} position residue. This difference is accommodated by Hans-LanM having a Gly at the 4^{th} position in EF hands 2 and 3, whereas Mex-LanM has a Gly at the 6^{th} position, and CaM has Gly residues at both positions (FIG. 13b).

FIG. 23 shows spectroscopic estimation of coordinated solvent molecules (q) in Eu^{III}₂-Hans-LanM using the Horrocks method. The luminescence lifetime of Eu^{III} complexes is empirically correlated with q. Triangles: values for the luminescence decay time constant (τ, in ms, left y-axis). By way of comparison, τ_{H2O}=1.24 ms for Hans-LanM but only 0.404 ms for Mex-LanM. Circles: 1/τ values (in ms⁻¹, right y-axis). The equation of the fit of 1/τ vs. mol fraction D₂O is used to determine q. The uncertainty in q is taken to be ±0.5. The q value of 0.11 is consistent with the absence of coordinated solvent in the Hans-LanM crystal structures. Conditions: 20 μM Hans-LanM, 40 μM Eu^{III}, 25 mM HEPES, 75 mM NaCl, pH 7.0. Each data point is the mean±s.d. for two independent samples.

FIG. 24 shows the extended hydrogen bonding networks in the metal-binding sites of Hans- and Mex-LanMs. a, The metal sites of Hans-LanM display extensive hydrogen bonding between ligands and several backbone amides, as well as the sidechain of the Thr residue at the 7^{th} position. In EF3, this network is extended further via interaction of Asp85 and Glu91 with Arg100 of the adjacent monomer. b, The metal sites of Mex-LanM feature similar hydrogen bonding patterns, but solvent molecules w1 and w2 take the place of Glu69. c, Enlarged view of the backbone flip that allows for hydrogen bonding interactions between the 1^{st} position Asp and Asn residues and a mainchain amide and carbonyl in Mex-LanM and Hans-LanM, respectively.

FIG. 25 shows a titration of R100K-Hans-LanM with La^{III}, Nd^{III}, and Dy^{III}, using CD spectroscopy (pH 5.0). Fitted parameters are summarized in Table 7. Results with Nd^{III} and Dy^{III} (K_{d,app} and change in molar ellipticity) are essentially identical to those with the wild-type Hans-LanM protein; with La^{III}, a 2-fold weaker K_{d,app} and larger change in molar ellipticity are observed. Conditions: 15 μM protein, 20 mM acetate, 100 mM KCl, pH 5.0, 10 mM EDTA (La,Nd) or EGTA (Dy), 0-10 mM RE^{III}. Each data point is the mean±s.d. for two independent samples.

FIG. 26 shows SEC-MALS traces of R100K-Hans-LanM, as apoprotein and metalated with 3 equiv. La^{III}. The apoprotein migrates similarly to Mex-LanM and wild-type Hans-LanM (FIG. 7), indicative of a disordered protein. The La^{III} complex migrates as a single, sharp peak. Both samples were determined to have similar weight-averaged molar masses by MALS corresponding to a monomer (see Table 8). Conditions: 3 mg/mL protein, 30 mM MOPS, 100 mM KCl, pH 7.0.

FIG. 27 shows Hansschlegelia quercus LanM and LanM proteins predicted to dimerize are phylogenetically distinct from other LanMs. The Bayesian phylogeny was constructed using a site-homogeneous model based on the Whelan and Goldman matrix with invariant sites and four distinct gamma categories (WAG+I+Γ₄) under a strict clock with minimum sequence length of 106 amino acids. The monophyletic group including members of the Hans cluster is highlighted grey. Node values indicate posterior probabilities based on 10,000,000 iterations with a burn-in of 25%. The scale bar represents 0.1 changes per amino acid position. The LanM core sequence alignment used to construct the phylogeny is colored in the Zappos scheme. The four EF-hand domains are denoted with a line at the bottom of the alignment, and residues associated with dimer interaction with an asterisk. The Bayesian phylogeny constructed from this alignment supports the network structure, where the Hans cluster is represented as a monophyletic group and placed farther apart from other sequences. In addition, the topology of the Hans cluster in the phylogenetic tree corresponds to the proximity seen in the network (FIG. 1a and FIG. 50). In this alignment, R100 is in position 87, denoted with an asterisk along with the other three residues in EF-hand 3 involved in the dimerization interface (i.e., D72, E78, and D80). All four residues involved in dimerization are conserved in the Hans cluster LanMs, suggesting that these proteins all form dimers. Only a single LanM outside of the Hans cluster (an unclassified Hyphomicrobiaceae) has an Arg residue at position 87. The EF3 sequence in this ortholog lacks several Ln^{III} ligands, but EF2 features the D₁₁ residue that (in EF3) in Hans mediates interaction with the Arg, suggesting that this uncharacterized LanM may dimerize along a distinct interface. No sequences other than those included in this alignment contained a basic amino acid in position 87—instead most, like Mex-LanM, have an acidic residue (usually Glu). Additionally, a group of LanMs containing two cysteine residues, one near each terminus (in proximity to EF1 and EF4), was identified. Given that LanMs are periplasmic proteins, we propose that these proteins have a disulfide bond between these residues, which may provide added structural stability to LanM, but more evidence is required to ascertain the role of these residues.

FIG. 28 shows two views of EF-hand 3 of Dy^{III}-Hans-LanM with the 2Fₒ-F_{c} electron density map (gray mesh, contoured at 1.0 σ) and anomalous difference map (purple mesh, contoured at 3.0 σ) shown. a, Density associated with metal ligands is clear and no coordinated solvent is apparent. b, The hydrogen bonding network between EF3 and Arg100 of the adjacent monomer is also clearly visualized.

FIG. 29 shows the X-ray absorption edge of Dy-Hans-LanM detected by fluorescence excitation.

FIG. 30 shows anomalous diffraction datasets collected at the Lim edge (7793.5 eV) for Dy on Dy^{III}-Hans-LanM crystals supports assignment of the bound lanthanide as the HRE, Dy. Anomalous difference electron density map is shown in purple mesh (contoured at 4.0σ) for representative metal binding sites in chain A. In all four EF hands, we observe significantly more intense anomalous difference electron density map peaks above the L_{III} edge (Table 10). Interestingly, EF2 and EF3 exhibit the largest anomalous difference map peaks, perhaps reflecting the biochemical observation of only two high-affinity sites in this complex (FIG. 1d, FIG. 6).

FIG. 31 shows all four copies of the Arg100-EF3 hydrogen bonding network in the asymmetric unit of Dy^{III}-Hans-LanM show the same shift to monodentate coordination in Glu91 along with lengthening of the hydrogen bond between this residue and Arg100, from ˜2.9 Å in Lam^{III}-Hans-LanM to 3.2 Å in Dy^{III}-Hans-LanM. One of the Arg100-Asp93 hydrogen bonds also lengthens from 2.5 Å in La^{III}-Hans-LanM to 2.7 Å. The Arg100-Asp85 hydrogen bond compresses slightly from 3.2 Å in La^{III}-Hans-LanM to 2.8/2.9 Å in Dy^{III}-Hans-LanM. Although it is possible that forcing dimerization under the high-concentration conditions for crystallography may alter the interactions between monomers relative to those at lower concentration in solution, the La- and Dy-bound structures illustrate how the carboxylate shift of Glu91 can alter this second-sphere hydrogen bonding network, which would plausibly disfavor dimerization, an explanation strongly supported by characterization of the R100K variant (Table 8, FIGS. 25-27).

FIG. 32 shows the X-ray crystal structure of Nd^{III}-bound Mex-LanM, solved at 1.01 Å resolution. a, Overall structure of Mex-LanM. The Nd^{III} ion in EF4 is present due to the crystallization conditions (3.5 equiv. Nd^{III} and millimolar protein); prior biochemical analyses have shown that the weakest binding equivalent is with micromolar K_{d} and that the weak site is associated with EF4. b, 2Fₒ-F_{c} electron density map (gray mesh, contoured at 1.0 σ) and anomalous difference map (purple mesh, contoured at 3.0 σ) of EF3, showing the two solvent molecules coordinated to the Nd^{III} ion. The Nd^{III} ion is an aqua sphere, and solvent molecules are red spheres. c, Details of the four EF-hands. Metal coordination in EF1-3 is identical, with D₁, D₃, and the backbone CO of T₇ being monodentate ligands, D₅ and E₁₂ being bidentate ligands, and two water molecules (w1, w2) yielding 9-coordination. In EF4, the D₃ residue (Asp110) is bidentate. Because EF4 possesses an Asn at the 1^{st} position rather than an Asp, the non-coordinated sidechain N cannot hydrogen bond with the backbone, which may contribute to the lower affinity of this site.

FIG. 33 shows a comparison of hydrogen bonding networks connecting metal-binding sites to the exiting helices in La^{III}-Hans-LanM and Nd^{III}-Mex-LanM. These extensions of the backbone CO—HN hydrogen bonding networks within each helix to include the metal sites may contribute to the overall stabilization of the folded state of the proteins in the RE^{III}-LanM complexes. a, In La^{III}-Hans-LanM, although the E9 residues are bidentate ligands and therefore a directly analogous hydrogen bond cannot occur, Glu91 in EF3 is connected to the first backbone NH of the exiting helix via the hydrogen bonding network involving Arg100 and Asp93. The disruption of this network, and therefore a connection between the helix and the metal site, in the presence of the HREs may also contribute to the lower stability of the HRE-Hans-LanM complexes. b, In Nd^{III}-Mex-LanM, the D9 residues (Asp92 in EF3) directly connect the Glu95 NH from the exiting helix to Nd^{III}-coordinated solvent (w2). The different lengths of these two hydrogen bonds that would result from coordination of different REs may contribute to the selectivity trend in Mex-LanM.

FIG. 34 shows representative fluorescence emission intensity and wavelength (λₘₐₓ) changes from single measurements of wild-type Hans-LanM during titration with La^{III}. a, Emission spectra of Hans (λₑₓ=278 nm). La^{III} binding increases intensity 2-fold and shifts the λₘₐₓ from 343 nm to 333 nm. b, The excitation and emission wavelengths suggest the protein's two Trp residues, Trp79 and Trp95, near EF2 and EF3, primarily contribute to the spectra. Trp95 aligns with Tyr96 of Mex-LanM (FIG. 5), the fluorescence intensity of which has been shown to also be sensitive to metal binding and/or the associated protein conformational change. Conditions: 20 μM protein, 30 mM MOPS, 100 mM KCl, pH 7.0.

FIG. 35 shows representative Nd breakthrough curves for column-immobilized Hans-LanM and R100K-Hans-LanM. Experiments used 0.4 mM Nd^{III} in 7 mM homo-PIPES, pH 5.0. For Hans-LanM, 4.4±0.08 μmol/mL protein was immobilized, with a Nd adsorption capacity of 4.6±0.23 μmol/mL (1.06 equiv.). For R100K-Hans-LanM, 3.2±0.06 μmol/mL protein was immobilized, with a Nd adsorption capacity of 6.66±0.33 μmol/mL (2.08 equiv.). Each protein was immobilized once; the uncertainties in the immobilized protein represent s.d. from 3 replicate protein concentration determinations by BCA assay, and the uncertainties in the adsorption capacities were assumed to be 5% based on our previously reported LanM column experiments.

FIG. 36 shows Hans complexes with HREs more readily precipitate than LRE complexes. Pictures of (A) Hans+Dy (left) and La (right), (B) the samples from (A) after centrifugation, and (C) the samples from (A) after resolubilization with EDTA. This property may be adaptable into a separation procedure.

FIG. 37 shows the apparent K_{d} values of 15 μM Hans-LanM, compared with those of His-tagged Mex-LanM, monitored using CD spectroscopy. Buffer: 30 mM acetate, 100 mM KCl, 0-10 mM Ln^{III}, 10 mM EDTA or EGTA, pH 5.0.

FIG. 38 shows (Left) CD spectrum of Mex-LanM I42L/N108D/I115L (10 μM) shows that the protein exhibits additional alpha helical content in the apo state relative to the wild-type protein. (Right) CD spectrum of Mex-LanM I42L/N108D/I115L (5 μM) with was monitored at various EDTA-buffered free Nd^{III} ion concentrations using CD spectroscopy. Buffer: 20 mM acetate, 100 mM KCl, 0-10 mM Nd^{III}, 10 mM EDTA, pH 5.0. Note that there are some inaccuracies of ellipticity due to low concentration with a single accumulation, but the data suggest a K_{d,app} in the range of 1 pM. (Bottom) Determination of binding stoichiometry of wild-type Mex-LanM and its variants. Nd^{III} was titrated into the solution of protein and xylenol orange as previously described (Cotruvo et al., JACS 2018). Absorbance at 574 nm was plotted versus equivalents of Nd^{III}.

FIG. 39 shows CD titration curves (left) and [θ]_{222 nm} (right) of 2 μM Mex-LanM(A32D/A117K) monitored at various EDTA-buffered free Nd^{III} ion concentrations using CD spectroscopy. Buffer: 30 mM acetate, 100 mM KCl, 0-10 mM Nd^{III}, 10 mM EDTA, pH 5.0. The apparent K_{d} is 20±1 μM, with n=1.85±0.08. Because the K_{d,app} and points below it are at the lower limit of the EDTA buffering range (“% high solution” values of <2%, or 200 μM total Nd), metal binding to the 20 μM protein may be affecting the free metal concentrations but has not been accounted for in the calculations. Therefore, the true K_{d,app} values are slightly overestimated (i.e., affinities are underestimated) by this experiment.

FIG. 40 shows CD titration curves (left) and [θ]_{222 nm} (right) of 2 μM Mex-LanM(A32D/A117R) monitored at various EDTA-buffered free Nd^{III} ion concentrations using CD spectroscopy. Buffer: 30 mM acetate, 100 mM KCl, 0-10 mM Nd^{III}, 10 mM EDTA, pH 5.0. The apparent K_{d} is 22±1 μM, with n=1.51±0.08. Because the K_{d,app} and points below it are at the lower limit of the EDTA buffering range (“% high solution” values of <2%, or 200 μM total Nd), metal binding to the 20 μM protein may be affecting the free metal concentrations but has not been accounted for in the calculations. Therefore, the true K_{d,app} values are slightly overestimated (i.e., affinities are underestimated) by this experiment.

FIG. 41 shows time-resolved (left) and steady-state (right) fluorescence emission spectra comparing LanM_001 (Mex-LanM) and LanM_002 (Hans-LanM) bound to Eu^{III}. [Protein]=20 μM, [metal]=60 μM. Buffer: 30 mM MOPS, 100 mM KCl, pH 7.0. λₑₓ=280 nm, delay=150 s (left). λₑₓ=280 nm (right).

FIG. 42 shows time-resolved (left) and steady-state (right) fluorescence emission spectra comparing LanM_001 and LanM_002 bound to Tb^{III}. [Protein]=20 μM, [metal]=60 μM. Buffer: 30 mM MOPS, 100 mM KCl, pH 7.0. λₑₓ=300 nm, delay=100 μs (left). λₑₓ=280 nm (right).

FIG. 43 shows time-resolved fluorescence emission spectra comparing LanM_001 and LanM_002 bound to Sm^{III} (left) and Dy^{III} (right). [Protein]=20 μM, [metal]=60 μM. Buffer: 30 mM MOPS, 100 mM KCl, pH 7.0. λₑₓ=280 nm, delay=150 μs (left). λₑₓ=290 nm, delay=125 μs (right).

FIG. 44 shows (left) size exclusion chromatography of 20 μM LanM_012 in the presence of 4.0 equivalents of La^{III} or Dy^{III}. A sharp peak at a retention volume of ˜12.0 mL indicates a dimeric species for both conditions with an apparent molecular weight of ˜29.1 kDa. Buffer: 30 mM MOPS, 100 mM KCl, 5% glycerol, pH 7.0. (Right) Size exclusion chromatography of 400 μM LanM_012 in the absence of lanthanides. A peak at a retention volume of ˜14.0 mL suggests a monomeric species with an apparent molecular weight of ˜14.2 kDa. Buffer: 30 mM MOPS, 100 mM KCl, 5% glycerol, pH 7.0.

FIG. 45 shows LanM_012 binds 2.0 equivalents of La^{III} tightly in competition against the indicator xylenol orange, monitored at 575 nm. Protein: 2 μM Buffer: 20 mM MES, 100 mM KCl, 5 mM acetate, pH 6.0.

FIG. 46 shows titration of La^{III} to 20 μM LanM_012 at pH 5.0 followed using circular dichroism spectroscopy. The spectrum of the apo protein (0 Eq.) is suggestive of a well-folded protein with significant alpha-helical content. Addition of metal causes a small shift in the shape of the curve around 205 nm, suggestive of ordering of loop regions, and suggesting only minor change in the overall secondary structure of the protein. These preliminary results indicate that the apo form of the protein is already well-folded. Buffer: 20 mM acetate, 100 mM KCl, pH 5.0.

FIG. 47 shows CD spectra of LanM_012 in apo form or complexed with 2.0 equivalents La or Dy. Temperature was increased by 2° C./min and spectra were collected after each increase. The spectra at 16° C., 28° C., 74° C., and 84° C. were plotted for Apo (A), La (B), and Dy (C). The signal between 218 nm and 222 nm were averaged for each of the obtained spectra and plotted against temperature (D). The dotted line denotes the ellipticity value of the apoprotein at 222 nm at high temperature. Protein: 2 μM Buffer: 30 mM MOPS, 100 mM KCl, pH 7.0.

FIG. 48 shows (left) tryptophan fluorescence titration of 10 μM LanM_012, followed in the presence of various free metal concentrations. Buffer: 20 mM acetate, 100 mM KCl, 0-10 mM Ln^{III} pH 5.0. Free La^{III} and Dy^{III} concentrations were buffered using 10 mM EDTA and 10 mM EGTA, respectively. λₑₓ=280 nm. (Right) LanM_012 (20 μM) was metalated with 3.0 equivalents of La, Nd, or Dy, and tryptophan fluorescence at 333 nm was monitored in the presence of various citrate concentrations. Disassociation of the protein-metal complex induces a loss of tryptophan fluorescence intensity. Buffer: 20 mM acetate, 100 mM KCl, pH 5.0. λₑₓ=280 nm.

FIG. 49 shows time-resolved fluorescence emission spectra comparing apo LanM_012 and LanM_012-Ln₂ for Sm^{III} (A) and Dy^{III} (B). Steady-state fluorescence emission spectra comparing apo LanM_012 and LanM_012-Ln₂ for Eu^{III} (C) and Tb^{III} (D). [Protein]=20 μM, Buffer: 30 mM MOPS, 100 mM KCl, pH 7.0. λₑₓ=280 nm, delay=150 μs (A); λₑₓ=290 nm, delay=125 s (B); λₑₓ=280 nm (C); λₑₓ=280 nm (D).

FIG. 50 shows an expanded view of the inset from FIG. 1a (Hans cluster) including 20 sequences and 190 edges. The Hans cluster includes LanMs from bacteria from genera Hansschlegelia, Ancylobacter, Methylopila, Oharaeibacter, Starkeya, and Xanthobacter. Although these genera are restricted to this cluster, members at the family level are found dispersed throughout the network, including one Xanthobacteraceae and 42 Methylocystaceae.

FIG. 51 shows CD titrations of Hans-LanM with chelator-buffered solutions of (a) Ca^{II}, (b) Nd^{III}, and (c) Dy^{III}. Both Dy^{III} (up to 0.3 μM) and Ca^{II} (up to 5.5 mM) induce a similar, incomplete conformational change in the protein, relative to the conformational change induced by Nd^{III} and La^{III}. The data in the right panel of a is a representative titration from the 3 datasets used to generate the plot in the left panel. The data in b and c are representative titrations from the 3 datasets used to generate the plot in FIG. 1d. Conditions: 15 μM protein, 20 mM acetate, 100 mM KCl, 10 mM EDTA (for Ca and Nd titrations) or EGTA (for Dy titration), 0-10 mM metal ion. Each data point in (a, left panel) is the mean s.d. for three independent measurements.

FIG. 52 shows the X-ray crystal structure of La^{III}-bound Hans-LanM, solved at 1.8 Å resolution. a, Overall structure of the asymmetric unit, which consists of two Hans-LanM dimers and two citrate molecules from the crystallization solution. The structure of each monomer of the dimer is consistent with the NMR solution structure of Y^{III}-bound Mex-LanM with EF-hands 2 and 3 paired and EF-hands 1 and 4 paired. b-e, Details of metal coordination in the four EF-hands of La^{III}-Hans-LanM. The coordination spheres of the La^{III} ions in EF-hands 1, 2, and 3 are constituted by the side chain 06 of Ni (monodentate), the carboxylate side chains from D₃, D₅, E₉, and E₁₂ (all bidentate), and a backbone carbonyl from S₇ (EF1) or T₇ (EF2 and EF3), for a total coordination number of 10. All La^{III}-ligand distances are 2.5-2.7 Å. The crystal radius for 10-coordinate La^{III} is given as 1.41 Å by Shannon; given 1.26 Å as radius of 6-coord O², 2.57 Ø is estimated for the La^{III}-O distance, consistent with our results. The metal ion in EF4 was modeled as Na^{I} because of the shorter metal-ligand distances, lower coordination number, and the presence of sodium in the crystallization solution. Ca^{II} cannot be completely ruled out as it was present earlier in the protein purification; however, the protein was treated with Chelex at the end of the purification, and the crystallographic data were consistent with the Na^{I} assignment as determined by the CheckMyMetal server. This ion is coordinated in distorted pentagonal bipyramidal geometry by monodentate D₁, N₃, and D₅ sidechains, the bidentate E₁₂ sidechain, the backbone carbonyl of K113, and a single solvent molecule for a total coordination number of 7. The Na^{I}-protein ligand distances are 2.3-2.5 Å, with a solvent molecule at 2.7 Å. In the case of Mex-LanM, biochemical data and NMR spectroscopy have also supported EF4 as a poor lanthanide-binding site, and it was modeled without a metal ion in the NMR solution state structure.

FIG. 53 shows the X-ray crystal structure of Dy^{III}-bound Hans-LanM, solved at 1.4 Å resolution. a, One of the dimers in the asymmetric unit, comprising chains A and B. Note that EF4 is unexpectedly occupied with Dy^{III} while EF1 is occupied only in chain A. b, Overall structure of the asymmetric unit, which consists of two Hans-LanM dimers. Unlike the La^{III}-Hans-LanM structure, the two dimers—and the monomers within each dimer—display significant differences in Dy^{III}-Hans-LanM. EF2-4 are occupied by Dy^{III} in all chains, whereas EF1 is only occupied and ordered in chain A; in chains B and C, no metal ion is bound in the EF-hand, and in chain D, a Dy^{III} ion is bound but the first five residues of EF1 (N34-D38) could not be modeled. Our decision to model Dy^{III} into all four EF-hands is supported by anomalous diffraction datasets (Table 9-10, FIG. 29-30). The biochemical data suggest that, in solution, at least one Dy^{III} binding site is weak (see FIG. 1d and FIG. 6), and it is likely based on studies of Mex-LanM that EF2/3 are the tighter binding sites. This proposal is supported by the Dy anomalous data (Table 10), and the occupancy of weak metal-binding sites likely results from the high protein concentration used for crystallography. c, Details of metal coordination in the EF-hands of Dy^{III}-Hans-LanM. In the top row, the three different EF1 structures in the asymmetric unit are shown. Only in chain A is the EF1 metal site nearly identical to the sites in EF2 and EF3 (contrary to La^{III}-Hans-LanM, where EF1-3 sites are very similar, FIG. 52). In EF1 (chain A), EF2, and EF3, the coordinating ligands are the same as with La^{III}-Hans-LanM, except that the E9 residues (Glu42, Glu66, and Glu91) have shifted to monodentate coordination, resulting in 9-coordination. The lower coordination number with Dy^{III} is consistent with the lanthanide contraction and is observed with other ligands. The Dy^{III}-ligand distances are mostly 2.3-2.5 Å, ˜0.2 Å shorter than for La^{III}-Hans-LanM. Consistent with this observation, the crystal radius for 9-coordinate Dy^{III} is given as 1.22 Å by Shannon, 0.19 Å shorter than for 10-coordinate La^{III} (FIG. 52). The carboxylate shift of the 9^{th} position Glu residue is noteworthy as this position is important for gating affinity and selectivity in other EF-hand proteins. In EF4, Dy^{III} is 7-coordinate with pentagonal bipyramidal geometry, similar to the sodium site in La^{III}-Hans, but with slightly shorter metal-ligand distances (2.2-2.5 Å); again, these distances are consistent with the expectation for 7-coordinate Dy^{III}.

FIG. 54 shows spectrofluorometric titrations of RE-LanM (Hans-LanM, R100K-Hans-LanM, and Mex-LanM) complexes with citrate as a competitor, monitored by intrinsic protein fluorescence. Emission values are normalized to 1.0 for the fluorescence of the apoprotein. Note that the fluorescence intensity of Hans-LanM's Trp residues decreases going from the RE-bound to apo state (FIG. 34), whereas the intensity of Mex's Tyr residue increases going from the RE-bound to apo state. Initial conditions: 20 μM protein, 40 μM RE, 20 mM acetate, 100 mM KCl, pH 5.0, for all experiments, into which increasing concentrations of citrate were titrated. The citrate concentrations at which 50% of each metal is desorbed under these conditions ([citrate]1/2) are summarized in Table 11 and plotted in FIG. 4a. a, Hans-LanM. b, R100K-Hans-LanM. The compressed difference between the [citrate]_{1/2} values for La and Nd in wild-type vs. R100K-Hans-LanM illustrates the role of dimerization in enhancing affinity differences for the LREs, especially La^{III}. c, Mex-LanM. Nd and Dy data were previously reported. d, Comparison of the ratios of [citrate]_{1/2} value for Nd to that for Dy, for each protein, illustrating the greater Nd/Dy selectivity of Hans-LanM relative to Mex-LanM. The ratios for wild-type Hans-LanM and the R100K variant are not significantly different (p>0.05) by two-tailed t-test, suggesting that the hydrogen bonding network involving Arg100 does not contribute much to Nd^{III}/HRE selectivity, though it does impact La^{III} selectivity significantly (FIG. 4a). All data are shown as mean±s.d. (a-c) or s.e.m. (d) for data from 3 independent experiments.

FIG. 55 shows separation of a 95:5 mixture of Nd:Dy using immobilized Hans-LanM. The desorption scheme consisted of three stepped concentrations of malonate (30, 50, 90 mM; see right axis) followed by pH 1.5 (HCl). The results revealed that slightly lower purity Dy was generated using Hans-LanM compared to the R100K variant (83.6% vs 98% Dy purity at similar yield, respectively; compare to FIG. 4d). While similar selectivity profiles were observed for the immobilized proteins for La through Gd in equilibrium binding experiments with La-Dy, the selectivity pattern diverged at Tb (FIG. 4c). The selectivity difference between Hans-LanM and the R100K variant was confirmed by using a Nd/Dy binary system, as the uncertainties in the distribution factor determination for Dy in the 9-element RE group precluded the ability to distinguish small differences in the Dy/Nd separation factor between proteins (Tables 13-14). In this binary Nd/Dy experiment (Table 19), we determined a separation factor of 8.12±0.40 for Hans-LanM and 12.7±1.3 for the R100K variant, which is consistent with the improved Dy separation efficacy of R100K. While consistent with the values derived from the 9-element experiment, the results differ slightly from the equilibrium binding results with the free Hans-LanM and R100K-Hans-LanM proteins, which revealed similarly high selectivity for Nd over Dy (FIG. 4a,b), likely reflecting weaker LRE-induced dimerization in the R100K variant at the low protein concentration (20 μM) of the solution experiments with free protein. The La/Nd selectivity on-column is also distinct from that observed with the apparent K_{d} values of the free proteins (wild-type and R100K) in solution, although the experiments with free proteins utilized single element solutions and effects from mixed metal binding may impact the on-column data. The R100K variant is also better behaved on the column, as evidenced by the 2:1 RE:protein stoichiometry. One possible explanation for these results could be that immobilization interferes with dimerization; however, FIG. 2b shows the N- and C-termini of the Hans-LanM dimer, indicating that the C-termini are ˜20 Å from the nearest part of the dimer interface, suggesting that immobilization per se would not be expected to disrupt this interface. It must be considered, however, that a functional dimer would require two C-termini to be immobilized in close proximity, which is unlikely at the immobilization densities of our columns. Therefore, on balance, we suspect that the dimerization equilibrium is only applicable in a minority of protein units immobilized on the column. We posit that more fully exploiting the dimerization equilibrium in the column format would yield even more robust separations. The surest way to obtain homogeneous populations of dimers on-column would likely be to link two monomers together (e.g., with a polypeptide chain), tuning dimerization affinity through mutagenesis of the residues contributing to inter-monomer interactions, and immobilizing this dimer through a single attachment point. Dimerization could also be exploited in other separation formats. These directions are the subject of current efforts.

FIG. 56 shows SDS-PAGE from S75 gel filtration chromatography of LanM_013, showing high purity. The lanes are labeled with the fraction number.

FIG. 57 shows LanM_013 shows ˜2.0 equivalents of La(III) binding in titrations followed by UV-vis spectroscopy. 278 nm data is the top, 285 nm data is the bottom points. Buffer: 30 mM MOPS, 100 mM KCl, 5% glycerol, pH 7.0, 20 μM LanM_013.

FIG. 58 shows LanM_013-metal interactions were tracked by following protein tyrosine fluorescence. Conditions: 20 mM acetate, 100 mM KCl, 5% glycerol, pH 5.0, 20 μM protein.

FIG. 59 shows citrate was used as a competitor to remove bound La(III) from LanM_013 (3 equiv. La(III) added before starting the experiment). Conditions: 20 mM acetate, 100 mM KCl, 5% glycerol, pH 5.0, 20 μM protein.

FIG. 60 shows xylenol orange competition titration was used to assess metal binding of LanM_013 directly at pH 6.0. Conditions: 20 mM MES, 20 mM acetate, 100 mM KCl, chelex-treated, pH 6.0, 20 μM protein.

FIG. 61 shows the LanM_013 conformation change monitored using circular dichroism spectroscopy. Conditions: (pH 5.0) 20 mM acetate, 100 mM KCl, 5% glycerol, pH 5.0, 20 μM protein. (pH 7.0) 30 mM MOPS, 100 mM KCl, 5% glycerol, pH 7.0, 20 μM protein.

FIG. 62 shows affinity determination of LanM_013 at pH 5.0. Conditions: 10 μM LanM_013, 20 mM acetate, 100 mM KCl, 0-10 mM LaCl₃, 10 mM EDTA, pH 5.0 (left). 20 μM LanM_013, 20 mM acetate, 100 mM KCl, 0-10 mM DyCl₃, 10 mM EGTA, pH 5.0 (right). The apparent K_{d} values are 11 μM (La) and 96 μM (Dy). Both Hill coefficients are ˜1.5.

FIG. 63 shows steady-state emission spectra of various LanM-based sensors (5 μM) in the presence of two equivalents Nd(III) (A-C) or Yb(III) (D-F). (A) Mex-LanM(T90W) with Nd, (B) Hans-LanM(R100K) with Nd, (C) LanM_012 with Nd, (D) Mex-LanM(T90W) with Yb, (E) Hans-LanM(R100K) with Yb, (F) LanM_012 with Yb. Excitation 280 nm.

FIG. 64 shows determination of apparent K_{d}s of LanM_013 (10 μM) by CD spectroscopy, with free lanthanide ion concentrations buffered using EGTA. (A) A plot of [θ]_{222 nm} versus free Gd^{III} concentrations. A bi-phasic fit was used to determine the apparent dissociation constants (K_{d1,app}, K_{d2,app}) and Hill coefficients (n₁, n₂). (B) Plot of [θ]_{222 nm} versus free Dy^{III} concentrations. The single-phase fit was used to determine the apparent dissociation constant (K_{d,app}) and Hill coefficient (n). (C) Plot of [θ]_{222 nm} versus free Ho^{III} concentrations. The single-phase fit was used to determine the apparent dissociation constant (K_{d,app}) and Hill coefficient (n). Buffer: 20 mM acetate, 100 mM KCl, 0-10 mM Ln^{III}-EGTA, pH 5.0. Samples were equilibrated for 3 h prior to measurement.

FIG. 65 shows a selectivity profile of column-immobilized lanmodulin variants for the La-Dy REE group. LanM_001=Mex-LanM. LanM_002=Hans-LanM. Comparison of distribution values (log D) at equilibrium of total REE using the LanM column at pH 5. The right plot is the same as for the left, except it also includes LanM_002 for reference. The calculated separation factors are shown in the table below. Column dimensions: 5 cm×0.5 cm.

FIG. 66 shows a selectivity profile of column-immobilized lanmodulin variants for the Gd-Lu,Y REE group. LanM_001=Mex-LanM. LanM_002=Hans-LanM. Comparison of distribution values (log D) at equilibrium of total REE using the LanM column at pH 5. The right plot is the same as for the left, except it also includes LanM_002 for reference. The calculated separation factors are shown in the table below. Column dimensions: 5 cm×0.5 cm.

FIG. 67 shows characterization of variant group 1. (A) XO competition assay with titration of Nd(III), pH 6.1. The absorbance of XO at 574 nm was plotted versus Nd(III) equivalents. (B) The conformational change of LanM variants (20 μM) was monitored by CD at pH 5.0. The ellipticity at 222 nm was plotted versus Nd(III) equivalents.

FIG. 68 shows characterization of variant group 2. (A) XO competition assay with titration of Nd(III), pH 6.1. The absorbance of XO at 574 nm was plotted versus Nd(III) equivalents. (B) The conformational change of LanM variants (20 μM) was monitored by CD at pH 5.0. The ellipticity at 222 nm was plotted versus Nd(III) equivalents.

FIG. 69 shows characterization of variant group 3. (A) XO competition assay with titration of Nd(III), pH 6.1. The absorbance of XO at 574 nm was plotted versus Nd(III) equivalents. (B) The conformational change of LanM variants (20 μM) was monitored by CD at pH 5.0. The ellipticity at 222 nm was plotted versus Nd(III) equivalents.

FIG. 70 shows characterization of the “combo” variant group. (A) XO competition assay with titration of Nd(III), pH 6.1. The absorbance of XO at 574 nm was plotted versus Nd(III) equivalents. (B) The conformational change of LanM variants (20 μM) was monitored by CD at pH 5.0. The ellipticity at 222 nm was plotted versus Nd(III) equivalents.

FIG. 71 shows conformational change of LanM variant group 3 (5 μM) was monitored by CD at pH 5.0. The ellipticity at 222 nm was plotted versus Nd(III) free concentration. (A) A98G (B) A99G (C) A102G. Buffer: 20 mM acetate, 100 mM KCl, 10 mM Nd-EGTA, pH 5.0.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent, trivalent, and the like, radicals). Illustrative examples of groups include:

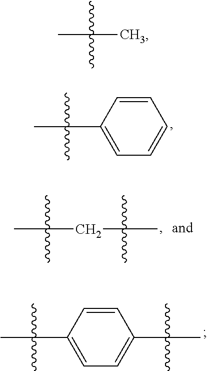

Amino acids and amino acid residues may be referred to herein by either their commonly known three letter symbols or by the one-letter symbols recommended by the IUPAC-IUB Biochemical Nomenclature Commission.

Examples of hydrophobic amino acid and hydrophobic amino acid residues include, but are not limited to, glycine, alanine, valine, leucine, isoleucine, proline, cysteine, phenylalanine, methionine, and tryptophan.

The present disclosure provides proteins that bind rare earth metals. Also provided are devices and kits comprising a protein of the present disclosure. Also provided are methods of using the proteins and devices.

In an aspect, the present disclosure provides proteins that bind metals (e.g., lanthanides and/or actinides). Other metal-binding proteins are disclosed in WO2020051274 and WO2023004333, which are incorporated herein by reference.

Wt Hans-LanM of the present disclosure may be one of the following peptides:

| Construct | Protein |
|---|---|
| Hans-LanM (full sequence, signal | MKLSLKAGAA ITAFVFAASP VLAASGADAL KALNKDNDDS |
| peptide underlined) | LEIAEVIHAG ATTFTAINPD GDTTLESGET KGRLTEKDWA |
| | RANKDGDQTL EMDEWLKILR TRFKRADANK DGKLTAAELD |
| | SKAGQGVLVM IMK (SEQ ID NO: 1) |
| Hans-LanM (lacking signal | MASGADAL KALNKDNDDS LEIAEVIHAG ATTFTAINPD |
| peptide, as expressed herein) | GDTTLESGET KGRLTEKDWA RANKDGDQTL EMDEWLKILR |
| | TRFKRADANK DGKLTAAELD SKAGQGVLVM IMK (SEQ ID |
| | NO: 2) |
| R100K-Hans-LanM (mutation | MASGADAL KALNKDNDDS LEIAEVIHAG ATTFTAINPD |
| underlined) | GDTTLESGET KGRLTEKDWA RANKDGDQTL EMDEWLKILK |
| | TRFKRADANK DGKLTAAELD SKAGQGVLVM IMK (SEQ ID |
| | NO: 4) |
| Hans-LanM-Cys (for | MASGADAL KALNKDNDDS LEIAEVIHAG ATTFTAINPD |
| immobilization) | GDTTLESGET KGRLTEKDWA RANKDGDQTL EMDEWLKILR |
| | TRFKRADANK DGKLTAAELD SKAGQGVLVM IMKGSGC (SEQ |
| | ID NO: 5) |
| R100K-Hans-LanM-Cys (for | MASGADAL KALNKDNDDS LEIAEVIHAG ATTFTAINPD |
| immobilization) | GDTTLESGET KGRLTEKDWA RANKDGDQTL EMDEWLKILK |
| | TRFKRADANK DGKLTAAELD SKAGQGVLVM IMKGSGC (SEQ |
| | ID NO: 6) |

A protein of the present disclosure may be of various lengths. For example, a protein of the present disclosure has 65 to 160 amino acid residues, including all integer amino acid values and ranges therebetween. For example, the protein has a molecular weight of around 8 kDa to 14 kDa, including all 0.1 Da values and ranges therebetween (e.g., −12 kDa). A protein of the present disclosure comprises at least one segment where one or more rare earth metals can bind. In various examples, the segment has at least 70% homology (e.g., at least 70%, at least 75%, at least 80%, at least 85%, at least 90%, or at least 95% homology) with the sequence of Hansschlegelia quercus LanM, which may be referred to as Hans-LanM. In various other examples, the protein is truncated. For example, the protein is truncated at the N-terminus via deletion of the first 10, 20, 30, or 40 residues of the full translated sequence. In another example, the protein is truncated at the C-terminus via deletion of the last 10, 20, 30, or 40 residues of the full translated sequence. In various examples of truncated sequences, EF hands 2 and 3 remain, as well as the majority of the hydrophobic core of the protein.

Suitable Hans-LanM proteins and LanM proteins of the present disclosure include the wild type H. quercus LanM protein, or orthologs from other organisms having at least two EF hand motifs, with at least one EF hand motifs having at least 3 carboxylate residues, and at least 2 of the EF hand motifs being separated by a space of 10-15 residues. Reference herein will be made generally to “lanmodulin,” “LanM” or “LanM protein” and should be understood to include the wild type and orthologs described herein. “LanM” can include full proteins having one or more LanM units or portions thereof comprising the one or more LanM units. LanM units include at least two EF hand motifs, with at least one EF hand motifs having at least 3 carboxylate residues, and at least 2 of the EF hand motifs being separated by a space of 10-15 residues. For ease of reference, discussion will be made with reference to lanmodulin, LanM or LanM protein and should be understood to include both the full proteins and portions of full proteins having the suitable LanM unit.

Various substitutions may be made in a peptide or protein of the present disclosure. For example, one or more amino acid residues may be substituted with a different amino acid residue. The amino acid residue may be a canonical or non-canonical amino acid residue. For example, R100 may be substituted with an amino acid residue. For example, this arginine may be substituted with a lysine (e.g., R100K). Other suitable variants of Hans-LanM include, but are not limited to, M92L, M92A, M92D, A44N, A44S, A44T, D93N, and D93 Å. Also included are proteins having least 70% homology (e.g., at least 70%, at least 75%, at least 80%, at least 85%, at least 90%, or at least 95% homology) to any of the foregoing variants. The residue number corresponds to the residue number when the 23-residue signal peptide is present. For proteins that lack the signal peptide, the first residue of these proteins is residue 24, after cleavage of the N-terminal methionine.

In various embodiments, a protein of the present disclosure has a residue suitable for immobilization onto a substrate. The residue may be part of a large sequence comprising 2 to 10 amino acid residues. For example, the residue comprises a functional group that chemically reacts with another functional group on the substrate such that the residue (and thus protein) is covalently attached to the substrate. For example, the substrate may comprise a maleimide group that can react with a nucleophilic group, such as the thiol of a cysteine or amine of a lysine. Other suitable chemistries (e.g., Click chemistry) are known in the art and may be used. For example, the substrate may be a resin or bead comprising a functional group that can react with the residue of the LanM protein. For example, the functional group may be a maleimide, alkyne, or azide.

In various examples, a protein or peptide of the present disclosure capable of binding with a metal/metal ion (e.g., a protein or peptide of present disclosure capable of dimerizing upon contact with a metal/metal ion) may comprise 4 EF hand motifs (e.g., a first EF hand motif, a second EF hand motif, a third EF hand motif, and a fourth EF hand motif), each EF hand motif comprising 11, 12, or 13 amino acid residues (e.g., 12 amino acid residues). Each EF hand motif is separated by 12 or 13 amino acid residues, where each amino acid residue is any canonical amino acid residue and at least one amino acid residue is a hydrophobic amino acid residue. In the case of the third EF hand motif (i.e., EF3) and the fourth EF hand motif (i.e., EF4), they are separated by the sequence (X)₅-R-(X)₆, where each X is any canonical amino acid residue. When the EF hand motif has 12 amino acid residues, the motif may have the following sequence: X¹-X²-X³-X⁴-X⁵-X⁶-X⁷-X⁸-X⁹-X¹⁰-X¹¹-E. For the first EF hand motif (i.e., EF1), the second EF hand motif (i.e., EF2), and EF4, X¹ is D or N; X³ is D, N, or E; X⁵ is D, N, or E; X⁸ is a hydrophobic residue; X⁹ is D, E, or T; X¹⁰ is a hydrophobic residue; and X², X⁴, X⁶, X⁷, and X¹¹ are each independently any canonical amino acid residue. In various examples, X⁷ of EF1, EF2, and/or EF4 is T or S. For EF3, X¹ is N; X³ is D; X⁴ is G or A; X⁵ is D or N; X⁷ is T or S; X⁸ is a hydrophobic residue, X⁹ is E; X¹⁰ is a hydrophobic residue; X¹¹ is D; and X² an X⁶ are each independently any canonical amino acid residue (e.g., N-X²-D-X⁴-X⁵-X⁶-X⁷-X⁸-E-X¹⁰-D-E (SEQ ID NO:97). In various examples, X⁴ of EF3 is A. In various examples, X⁸ of EF3 is L. In various examples, X¹⁰ of EF3 is L, I, or M. Without intending to be bound by any particular theory, it is considered that the dimerization strength of such a protein depends on the identity of the metal ion bound, where dimers form preferentially in the presence of a trivalent rare earth element or actinide. A protein having this sequence may be connected to another protein of the present disclosure via a peptide linker as described herein.

In various examples, a protein or peptide of the present disclosure may have enhanced REE/REE selectivity. The selectivity may be between light rare earth metals and heavy rare earth metals, and more than that of M. extorquens lanmodulin. Such a protein may comprise 4 EF hand motifs (e.g., a first EF hand motif, a second EF hand motif, a third EF hand motif, and a fourth EF hand motif), each EF hand motif comprising 11, 12, or 13 amino acid residues and each EF hand motif is separated by 12 or 13 amino acid residues, where each residue is a canonical residue and at least one amino acid residue is a hydrophobic amino acid residue. When the EF hand motif has 12 amino acid residues, the motif may have the following sequence:

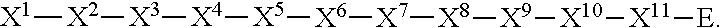

For the first EF hand motif (i.e., EF1) and fourth EF hand motif (i.e., EF4), X¹ is D or N; X³ is D, N, or E; X⁵ is D, N, or E; X⁸ is a hydrophobic residue; X⁹ is D, E, or T; X¹⁰ is a hydrophobic residue; and X², X⁴, X⁶, X⁷, and X¹¹ each independently any canonical amino acid residue. In various examples, X⁷ of EF1 and/or EF4 is T or S. For EF2, X¹ is N; X³ is D; X⁵ is D; X⁷ is T or S; X⁸ is a hydrophobic residue; X⁹ is E; X¹² is E; and X², X⁴, X⁶, X¹⁰, and X¹¹ are each independently any canonical amino acid residue (e.g., N-X²-D-X⁴-D-X⁶-X⁷-X⁸-E-X¹⁰-X¹¹-E (SEQ ID NO:95). In various examples, X⁸ of EF2 is L, I M, or V. For EF3, X¹ is D; X³ is D; X⁵ is D; X⁶ is G; X⁷ is T or S; X⁸ is a hydrophobic residue; X⁹ is D; and X², X⁴, X¹⁰, and X¹¹ are each independently any canonical residue (e.g., D-X²-D-X⁴-D-G-X⁷-X⁸-D-X¹⁰-X¹¹-E (SEQ ID NO:96). In various embodiments, X⁸ of EF3 is L, I, M, or V. At least one X² of either EF2 or EF3 is P. A protein having this sequence may be connected to another protein of the present disclosure via a peptide linker as described herein.

In various embodiments, the second EF hand has the following sequence:

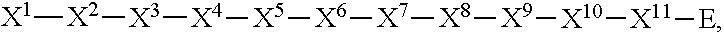

where:
X¹ is D or N;
X² is any canonical amino acid;
X³ is D, N, or E;
X⁴ is any canonical amino acid;
X⁵ is D, N, or E;
X⁶ is any canonical amino acid;
X⁷ is any canonical amino acid;
X⁸ is a hydrophobic residue;
X⁹ is independently a D, E, or T;
X¹⁰ is a hydrophobic residue; and
X¹¹ is any canonical amino acid.
   In various other embodiments, the second EF had has the following sequence:

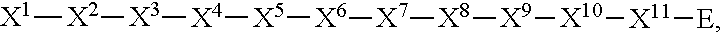

where:
X¹ is N;
X² is any canonical amino acid;
X³ is D;
X⁴ is any canonical amino acid;
X⁵ is D;
X⁶ is any canonical amino acid;
X⁷ is T or S;
X⁸ is a hydrophobic residue;
X⁹ is E;
X¹⁰ is any canonical amino acid; and
X¹¹ is any canonical amino acid.

In various embodiments, X² of the second EF hand is P. In various embodiments X⁷ is T or S. In various embodiments, X⁸ is L, I, M, or V

In various embodiments, the third EF hand has the following sequence:

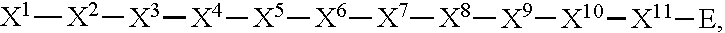

where:
X¹ is N;
X² is any canonical amino acid;
X³ is D;
X⁴ is G or A;
X⁵ is D or N;
X⁶ is any canonical amino acid;
X⁷ is T or S;
X⁸ is a hydrophobic residue;
X⁹ is E;
X¹⁰ is a hydrophobic residue; and
X¹¹ is D.

In various other embodiments, the third EF hand has the following sequence:

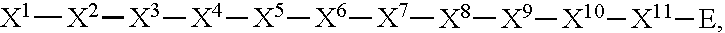

where:
X¹ is D;
X² is any canonical amino acid;
X³ is D;
X⁴ is D;
X⁵ is D;
X⁶ is G;
X⁷ is T or S;
X⁸ is a hydrophobic residue;
X⁹ is D;
X¹⁰ is any canonical amino acid; and
X¹¹ is any canonical amino acid.

In various embodiments, X² is P. In various embodiments, X⁴ is A. In various embodiments X⁸ is L, I, M, or V. In various embodiments, X¹⁰ is L, I, or M.

A protein of the present disclosure may comprise various EF1 hands. Examples of EF1 hands include, but are not limited to, NKDNDDSLEIAE (SEQ ID NO:51), NKDKDSTVEIVE (SEQ ID NO:52), DPDKDGTIDLNE (SEQ ID NO:53), DPDMDNALTLEE (SEQ ID NO:54), DPDKDGTIDLKE (SEQ ID NO:55), DPDKDGTLDLKE (SEQ ID NO:56), NPDHIDGTIDWRE (SEQ ID NO:57), DPDGDGAMTLGE (SEQ ID NO:58), NKDNDDSLEAAE (SEQ ID NO:59), NKDNDDSLEVAE (SEQ ID NO:60), NKDNDDSLEINE (SEQ ID NO:61), NKDNDDSLEISE (SEQ ID NO:62), NKDNDDSLEITE (SEQ ID NO:63), and NKDNDDSLQIAE (SEQ ID NO:64).

A protein of the present disclosure may comprise various EF2 hands. Examples of EF2 hands include, but are not limited to, NPDGDTTLESGE (SEQ ID NO:65), NPDKDKTLEAAE (SEQ ID NO:66), NPDGDGTLEVKE (SEQ ID NO:67), NTDDDNTLEADE (SEQ ID NO:68), DPDKDGTLDAKE (SEQ ID NO:69), DPDHIDGTLDMKE (SEQ ID NO:70), NKDGDITLELDE (SEQ ID NO:71), and NPDGDTTLQSGE (SEQ ID NO:72).

A protein of the present disclosure may comprise various EF3 hands. Examples of EF3 hands include, but are not limited to, NKDGDQTLEMDE (SEQ ID NO:73), NKDGDKTLELDE (SEQ ID NO:74), DPDNDGTLDMQE (SEQ ID NO:75), DPDDDGSLDMAE (SEQ ID NO:76), DPDNDGTLDKKE (SEQ ID NO:77), NPDRDGKLDKHE (SEQ ID NO:78), DLIKGRGISLGE (SEQ ID NO:79), NKDGDQTLELDE (SEQ ID NO:80), NKDGDQTLEADE (SEQ ID NO:81), NKDGDQTLEDDE (SEQ ID NO:82), NKDGDQTLEMAE (SEQ ID NO:83), NKDGDQTLEMNE (SEQ ID NO:84), and NKDGDQTLQMDE (SEQ ID NO:85).

A protein of the present disclosure may computer various EF4 hands. Examples of EF4 hands include, but are not limited to, DANKDGKLTAAE (SEQ ID NO:86), DANKDGKLTEAE (SEQ ID NO:87), NPDNDGTVDEKE (SEQ ID NO:88), NPDGDDTIESDE (SEQ ID NO:89), NPDNDGTIDKRE (SEQ ID NO:90), DPDNDGTLDARE (SEQ ID NO:91), NPDKDGTIDCRE (SEQ ID NO:92), NPDKDHTIECDE (SEQ ID NO:93), DPDNDGTIDARE (SEQ ID NO:94), and NPDNDGTIDARE (SEQ ID NO:98).

Examples of proteins of the present disclosure include, but are not limited to:

>Hans-LanM (full protein sequence, signal peptide underlined)
(SEQ ID NO: 1)
MKLSLKAGAA ITAFVFAASP VLAASGADAL KALNKDNDDS LEIAEVIHAG
ATTFTAINPD GDTTLESGET KGRLTEKDWA RANKDGDQTL EMDEWLKILR
TREKRADANK DGKLTAAELD SKAGQGVLVM IMK
>Hans-LanM (with signal peptide removed, as expressed herein)
(SEQ ID NO: 2)
MASGADAL KALNKDNDDS LEIAEVIHAG ATTFTAINPD GDTTLESGET
KGRLTEKDWA RANKDGDQTL EMDEWLKILR TREKRADANK DGKLTAAELD
SKAGQGVLVM IMK
>Hans-LanM(R100K) (with signal peptide removed, as expressed in this study, substitution
underlined)
(SEQ ID NO: 4)
MASGADAL KALNKDNDDS LEIAEVIHAG ATTFTAINPD GDTTLESGET
KGRLTEKDWA RANKDGDQTL EMDEWLKILK TRFKRADANK DGKLTAAELD
SKAGQGVLVM IMK
>Hans-LanM-Cys (for immobilization)
(SEQ ID NO: 5)
MASGADAL KALNKDNDDS LEIAEVIHAG ATTFTAINPD GDTTLESGET
KGRLTEKDWA RANKDGDQTL EMDEWLKILR TRFKRADANK DGKLTAAELD
SKAGQGVLVM IMKGSGC
>Hans-LanM(R100K)-Cys (for immobilization)
(SEQ ID NO: 6)
MASGADAL KALNKDNDDS LEIAEVIHAG ATTFTAINPD GDTTLESGET
KGRLTEKDWA RANKDGDQTL EMDEWLKILK TREKRADANK DGKLTAAELD
SKAGQGVLVM IMKGSGC
>LanM_012 (Xanthobacter flavus, with signal peptide removed)
(SEQ ID NO: 7)
MLTGKEFLRKYNKDKDSTVEIVEAIDLGTKVFKAINPDKDKTLEAAETKGRLSDEDWAQENK
DGDKTLELDEWLIIVRKRENDADANKDGKLTEAELDAPAGQQLILLIAK
>LanM_013 (expressed construct; signal peptide removed and N-terminal Met added, EF
hands underlined):
(SEQ ID NO: 8)
MGKAADAIQALDPDKDGTIDLNEAKAGAKAVFEKINPDGDGTLEVKELKGRLTKKELDAADP
DNDGTLDMQEYEAVVTKQFELANPDNDGTVDEKELKTKEGKKLLKLIY
>LanM_013 (full-length sequence from Methyloligella sp. GL2, with signal peptide,
underlined):
(SEQ ID NO: 9)
MAAILTIAGAVTVAAGGAAFAGKAADAIQALDPDKDGTIDLNEAKAGAKAVFEKINPDGDGT
LEVKELKGRLTKKELDAADPDNDGTLDMQEYEAVVTKQFELANPDNDGTVDEKELKTKEGKK
LLKLIY
>Methyloligella halotolerans (expected to behave similarly to LanM_013; sequence with
signal peptide removed)
(SEQ ID NO: 10)
MADAEISDTMKVVDPDMDNALTLEEAQAAGAKVFKKLNTDDDNTLEADELKGRVSERQLKKA
DPDDDGSLDMAEYEALIKKRFEAANPDGDDTIESDELETKKGKKLLELIQE
>Mex-LanM-A32D/A117K (mutations underlined)
(SEQ ID NO: 11)
MAPTTTTKVDIDAFDPDKDGTIDLKEALAAGSAAFDKLDPDKDGTLDAKELKGRVSEADLKK
LDPDNDGTLDKKEYLAAVEAQFKAANPDNDGTIDKRELASPAGSALVNLIR
>Mex-LanM-A32D/A117R (mutations underlined)
(SEQ ID NO: 12)
MAPTTTTKVDIDAFDPDKDGTIDLKEALAAGSAAFDKLDPDKDGTLDAKELKGRVSEADLKK
LDPDNDGTLDKKEYLAAVEAQFKAANPDNDGTIDRRELASPAGSALVNLIR
>Mex-LanM-I42L/N108D/1115L (mutations underlined)
(SEQ ID NO: 13)
MAPTTTTKVDIAAFDPDKDGTLDLKEALAAGSAAFDKLDPDKDGTLDAKELKGRVSEADLKK
LDPDNDGTLDKKEYLAAVEAQFKAADPDNDGTLDARELASPAGSALVNLIR
>LanM_011 (Hyphomicrobium, with signal peptide removed)
(SEQ ID NO: 14)
MGHHNCKAEMAYINPDHDGTIDWREARRAAVRLFHKLDPDHDGTLDMKEVRGRVGILSFARE
NPDRDGKLDKHEWLALVKHRFHRANPDKDGTIDCRELHSLAGRKL.LRVIM
>Unclassified Hyphomicrobium (signal peptide removed; appears to only have 3 functional
EF hands)
(SEQ ID NO: 15)
MGHRSAKAHPSCPALNAIDPDGDGAMTLGEAKRAAIKTEMKLNKDGDITLELDELGGRMSAA
AFAQADLIKGRGISLGEYLIEVRRRFKWANPDKDHTIECDELHSKYGRLLARLLK
>HansR100K-L1
(SEQ ID NO: 16)
MASGADALKALNKDNDDSLEIAEVIHAGATTFTAINPDGDTTLESGETKGRLTEKDWARANK
DGDQTLEMDEWLKILKTRFKRADANKDGKLTAAELDSKAGQGVLVMIMKGGSGGSGGSGGSG
GSGGSASGADALKALNKDNDDSLEIAEVIHAGATTFTAINPDGDTTLESGETKGRLTEKDWA
RANKDGDQTLEMDEWLKILKTRFKRADANKDGKLTAAELDSKAGQGVLVMIMK
>HansR100K-L2
(SEQ ID NO: 17)
MASGADALKALNKDNDDSLEIAEVIHAGATTFTAINPDGDTTLESGETKGRLTEKDWARANK
DGDQTLEMDEWLKILKTRFKRADANKDGKLTAAELDSKAGQGVLVMIMKGGSGGSGGSGGSG
GSGGSGGSGGSGGSGGSASGADALKALNKDNDDSLEIAEVIHAGATTFTAINPDGDTTLESG
ETKGRLTEKDWARANKDGDQTLEMDEWLKILKTRFKRADANKDGKLTAAELDSKAGQGVLVM
IMK
>HansR100K-L3
(SEQ ID NO: 18)
MASGADALKALNKDNDDSLEIAEVIHAGATTFTAINPDGDTTLESGETKGRLTEKDWARANK
DGDQTLEMDEWLKILKTRFKRADANKDGKLTAAELDSKAGQGVLVMIMKGGSGGSGGSGGSG
GSGGSGGSGGSGGSGGSGGSGGSASGADALKALNKDNDDSLEIAEVIHAGATTFTAINPDGD
TTLESGETKGRLTEKDWARANKDGDQTLEMDEWLKILKTRFKRADANKDGKLTAAELDSKAG
QGVLVMIMK
>HansR100K-L4
(SEQ ID NO: 19)
MASGADALKALNKDNDDSLEIAEVIHAGATTFTAINPDGDTTLESGETKGRLTEKDWARANK
DGDQTLEMDEWLKILKTRFKRADANKDGKLTAAELDSKAGQGVLVMIMKGGSGGSGGSGGSG
GSGGSGGSGGSGGSGGSGGSGGSGGSGGSASGADALKALNKDNDDSLEIAEVIHAGATTFTA
INPDGDTTLESGETKGRLTEKDWARANKDGDQTLEMDEWLKILKTRFKRADANKDGKLTAAE
LDSKAGQGVLVMIMK
>HansR100K-L5
(SEQ ID NO: 20)
MASGADALKALNKDNDDSLEIAEVIHAGATTFTAINPDGDTTLESGETKGRLTEKDWARANK
DGDQTLEMDEWLKILKTRFKRADANKDGKLTAAELDSKAGQGVLVMIMKGSGGSGAEAAAKE
AAAKAGGSGGSAEAAAKEAAAKAGSGGSGASGADALKALNKDNDDSLEIAEVIHAGATTFTA
INPDGDTTLESGETKGRLTEKDWARANKDGDQTLEMDEWLKILKTRFKRADANKDGKLTAAE
LDSKAGQGVLVMIMK
>Mex-LanM-G51A (mutations underlined)
(SEQ ID NO: 21)
MAPTTTTKVDIAAFDPDKDGTIDLKEALAAASAAFDKLDPDKDGTLDAKELKGRVSEADLKK
LDPDNDGTLDKKEYLAAVEAQFKAANPDNDGTIDARELASPAGSALVNLIR
>Mex-LanM-A98G (mutations underlined)
(SEQ ID NO: 22)
MAPTTTTKVDIAAFDPDKDGTIDLKEALAAGSAAFDKLDPDKDGTLDAKELKGRVSEADLKK
LDPDNDGTLDKKEYLGAVEAQFKAANPDNDGTIDARELASPAGSALVNLIR
>Mex-LanM-A99G (mutations underlined)
(SEQ ID NO: 23)
MAPTTTTKVDIAAFDPDKDGTIDLKEALAAGSAAFDKLDPDKDGTLDAKELKGRVSEADLKK
LDPDNDGTLDKKEYLAGVEAQFKAANPDNDGTIDARELASPAGSALVNLIR
>Mex-LanM-V100G (mutations underlined)
(SEQ ID NO: 24)
MAPTTTTKVDIAAFDPDKDGTIDLKEALAAGSAAFDKLDPDKDGTLDAKELKGRVSEADLKK
LDPDNDGTLDKKEYLAAGEAQFKAANPDNDGTIDARELASPAGSALVNLIR
>Mex-LanM-A102G (mutations underlined)
(SEQ ID NO: 25)
MAPTTTTKVDIAAFDPDKDGTIDLKEALAAGSAAFDKLDPDKDGTLDAKELKGRVSEADLKK
LDPDNDGTLDKKEYLAAVEGQFKAANPDNDGTIDARELASPAGSALVNLIR
>Hans-LanM-143A (mutation underlined)
(SEQ ID NO: 26)
MASGADALKALNKDNDDSLEAAEVIHAGATTFTAINPDGDTTLESGETKGRLTEKDWARANK
DGDQTLEMDEWLKILRTRFKRADANKDGKLTAAELDSKAGQGVLVMIMK
>Hans-LanM-I43V (mutation underlined)
(SEQ ID NO: 27)
MASGADALKALNKDNDDSLEVAEVIHAGATTFTAINPDGDTTLESGETKGRLTEKDWARANK
DGDQTLEMDEWLKILRTRFKRADANKDGKLTAAELDSKAGQGVLVMIMK
>Hans-LanM-A44N (mutation underlined)
(SEQ ID NO: 28)
MASGADALKALNKDNDDSLEINEVIHAGATTFTAINPDGDTTLESGETKGRLTEKDWARANK
DGDQTLEMDEWLKILRTRFKRADANKDGKLTAAELDSKAGQGVLVMIMK
>Hans-LanM-A44S (mutation underlined)
(SEQ ID NO: 29)
MASGADALKALNKDNDDSLEISEVIHAGATTFTAINPDGDTTLESGETKGRLTEKDWARANK
DGDQTLEMDEWLKILRTRFKRADANKDGKLTAAELDSKAGQGVLVMIMK
>Hans-LanM-A44T (mutation underlined)
(SEQ ID NO: 30)
MASGADALKALNKDNDDSLEITEVIHAGATTFTAINPDGDTTLESGETKGRLTEKDWARANK
DGDQTLEMDEWLKILRTRFKRADANKDGKLTAAELDSKAGQGVLVMIMK
>Hans-LanM-147A (mutation underlined)
(SEQ ID NO: 31)
MASGADALKALNKDNDDSLEIAEVAHAGATTFTAINPDGDTTLESGETKGRLTEKDWARANK
DGDQTLEMDEWLKILRTRFKRADANKDGKLTAAELDSKAGQGVLVMIMK
>Hans-LanM-147V (mutation underlined)
(SEQ ID NO: 32)
MASGADALKALNKDNDDSLEIAEVVHAGATTFTAINPDGDTTLESGETKGRLTEKDWARANK
DGDQTLEMDEWLKILRTRFKRADANKDGKLTAAELDSKAGQGVLVMIMK
>Hans-LanM-M92L (mutation underlined)
(SEQ ID NO: 33)
MASGADALKALNKDNDDSLEIAEVIHAGATTFTAINPDGDTTLESGETKGRLTEKDWARANK
DGDQTLELDEWLKILRTRFKRADANKDGKLTAAELDSKAGQGVLVMIMK
>Hans-LanM-M92A (mutation underlined)
(SEQ ID NO: 34)
MASGADALKALNKDNDDSLEIAEVIHAGATTFTAINPDGDTTLESGETKGRLTEKDWARANK
DGDQTLEADEWLKILRTRFKRADANKDGKLTAAELDSKAGQGVLVMIMK
>Hans-LanM-M92D (mutation underlined)
(SEQ ID NO: 35)
MASGADALKALNKDNDDSLEIAEVIHAGATTFTAINPDGDTTLESGETKGRLTEKDWARANK
DGDQTLEDDEWLKILRTRFKRADANKDGKLTAAELDSKAGQGVLVMIMK
>Hans-LanM-D93A (mutation underlined)
(SEQ ID NO: 36)
MASGADALKALNKDNDDSLEIAEVIHAGATTFTAINPDGDTTLESGETKGRLTEKDWARANK
DGDQTLEMAEWLKILRTRFKRADANKDGKLTAAELDSKAGQGVLVMIMK
>Hans-LanM-D93N (mutation underlined)
(SEQ ID NO: 37)
MASGADALKALNKDNDDSLEIAEVIHAGATTFTAINPDGDTTLESGETKGRLTEKDWARANK
DGDQTLEMNEWLKILRTRFKRADANKDGKLTAAELDSKAGQGVLVMIMK
>Hans-LanM(3E9Q)
(SEQ ID NO: 38)
MASGADALKALNKDNDDSLQIAEVIHAGATTFTAINPDGDTTLQSGETKGRLTEKDWARANK
DGDQTLQMDEWLKILRTRFKRADANKDGKLTAAELDSKAGQGVLVMIMK
>Mex-LanM-N108D/A124G (mutations underlined)
(SEQ ID NO: 39)
MAPTTTTKVDIAAFDPDKDGTIDLKEALAAGSAAFDKLDPDKDGTLDAKELKGRVSEADLKK
LDPDNDGTLDKKEYLAAVEAQFKAADPDNDGTIDARELASPGGSALVNLIR
>Mex-LanM-N108D/A127G (mutations underlined)
(SEQ ID NO: 40)
MAPTTTTKVDIAAFDPDKDGTIDLKEALAAGSAAFDKLDPDKDGTLDAKELKGRVSEADLKKLDPDND
GTLDKKEYLAAVEAQFKAADPDNDGTIDARELASPAGSGLVNLIR
>Mex-LanM-N108D/A102G (mutations underlined)
(SEQ ID NO: 41)
MAPTTTTKVDIAAFDPDKDGTIDLKEALAAGSAAFDKLDPDKDGTLDAKELKGRVSEADLKK
LDPDNDGTLDKKEYLAAVEGQFKAADPDNDGTIDARELASPAGSALVNLIR
>Mex-LanM-N108D
(SEQ ID NO: 42)
MAPTTTTKVDIAAFDPDKDGTIDLKEALAAGSAAFDKLDPDKDGTLDAKELKGRVSEADLKK
LDPDNDGTLDKKEYLAAVEAQFKAADPDNDGTIDARELASPAGSALVNLIR

Without intending to be bound by any particular theory, it is considered that at least a portion of the LanM proteins of the present disclosure will dimerize with another LanM monomer upon contact with a rare earth metal. The LanM monomers of the dimer may be the same or different. It is considered that at least the following sequences will dimerize:

(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK
(Hans-LanM, without the signal peptide)
(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK
(Hans-LanM(R100K), without the signal peptide)
TGKEFLRKYNKDKDSTVEIVEAIDLGTKVFKAINPDKDKTLEAAETKG
RLSDEDWAQENKDGDKTLELDEWLIIVRKRFNDADANKDGKLTEAELD
APAGQQLILLIAK
(LanM_012, without the signal peptide).

Without intending to be bound by any particular theory, it is considered that the aforementioned sequences may dimerize with or without the signal peptide incorporated into the LanM sequence.

In various examples, two proteins of the present disclosure may be covalently conjugated together. For example, the two proteins may be conjugated via a peptide linker. For example, the linker may be a (GGS)n motif, where n is 2, 3, 4, 5, or 6. For example, the linker may be GGSGGSGGSGGSGGSGGS (SEQ ID NO:43). Examples of conjugated proteins include, but are not limited to:

>HansR100K-L1
(SEQ ID NO: 16)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSASGADALKALNKDNDDSL
EIAEVIHAGATTFTAINPDGDTTLESGETKGRLTEKDWARANKDGDQTL
EMDEWLKILKTRFKRADANKDGKLTAAELDSKAGQGVLVMIMK
>HansR100K-L2
(SEQ ID NO: 17)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSASGADA
LKALNKDNDDSLEIAEVIHAGATTFTAINPDGDTTLESGETKGRLTEKD
WARANKDGDQTLEMDEWLKILKTREKRADANKDGKLTAAELDSKAGQGV
LVMIMK
>HansR100K-L3
(SEQ ID NO: 18)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSGGSGGS
ASGADALKALNKDNDDSLEIAEVIHAGATTFTAINPDGDTTLESGETKG
RLTEKDWARANKDGDQTLEMDEWLKILKTRFKRADANKDGKLTAAELDS
KAGQGVLVMIMK
>HansR100K-L4
(SEQ ID NO: 19)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSGGSGGS
GGSGGSASGADALKALNKDNDDSLEIAEVIHAGATTFTAINPDGDTTLE
SGETKGRLTEKDWARANKDGDQTLEMDEWLKILKTRFKRADANKDGKLT
AAELDSKAGQGVLVMIMK
>HansR100K-L5
(SEQ ID NO: 20)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGSGGSGAEAAAKEAAAKAGGSGGSAEAAAKEAAAKA
GSGGSGASGADALKALNKDNDDSLEIAEVIHAGATTFTAINPDGDTTLE
SGETKGRLTEKDWARANKDGDQTLEMDEWLKILKTRFKRADANKDGKLT
AAELDSKAGQGVLVMIMK.

The aforementioned sequences are not intended to be limiting. In various examples, a conjugated protein of the present disclosure may be immobilized on a resin for use in a device of the present disclosure.

Without intending to be bound by any particular theory, it is considered that LanM_013, methyloligella halotolerans, and LanM_011 do not form dimers.

In an aspect, the present disclosure provides devices. The device comprises one or more proteins of the present disclosures.

Various devices may comprise a protein of the present disclosure. Non-limiting examples of devices include filters, membranes, sensors, handheld detector, plate reader, fluorimeter, biosensors, in-line monitors, and the like.

In an aspect, the present disclosure provides kits. The kits may provide one or more proteins of the present disclosure and/or one or more devices of the present disclosure. The kit may include instructions for use of the proteins or devices.

In an aspect, the present disclosure provides various methods of using the proteins and/or devices of the present disclosure. A method of the present disclosure may be for binding one or more lanthanides and/or actinides or for detecting and/or quantifying the amount of one or more lanthanides and/or actinides.

A method of using a protein and/or device of the present disclosure may be a method for binding one or more rare earth metals (e.g., lanthanides and/or actinides) in a sample. Binding may occur by contacting the sample with one or more proteins and/or devices of the present disclosure. The method may be performed on various types of samples. Examples of samples include, but are not limited to drinking water, wastewater, ground water, ash ponds, aqueous extract from contaminated soil, drainage (e.g., mine drainage, such as, for example, acidic mine drainage) or leachate (e.g., electronic waste leachate or leachate of an ore leachate). In various other examples, the sample is a solid sample. The method may be applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

Various lanthanides (e.g., lanthanide ions) and/or actinides (e.g., actinide ions) may be bound by a protein and/or device. Examples of lanthanides and actinides that may be bound include, but are not limited to, La, Ce, Pr, Nd, Pm, Sm, Eu, Gd, Tb, Dy, Ho, Er, Tm, Yb, Lu, Sc, Y, and ions thereof. In various examples, any lanthanide is detected. For example, the lanthanide is chosen from Tb, Gd, Ho, Eu, Dy, Sm, Nd, Yb, and ions thereof. In various examples, the lanthanide is Tb, Eu, Sm, Dy, or an ion thereof. The bound lanthanides and/or actinides may be the same or different. The concentration of the lanthanide and/or actinides in the sample may be less than 100 ppm (e.g., less than 90, 80, 70, 60, 50, 40, 30, 20, 10, 1, 0.1, or 0.05 ppm).

In various examples, the one or more lanthanides and/or actinides bound to the one or more proteins and/or devices may be isolated from the proteins and/or devices and recovered. The lanthanides and/or actinides may be unbound by lowering the pH below ˜2.5 or by adding a chelator (e.g., citrate, EDTA, EGTA, malonate, or the like). In various embodiments, if one or more different lanthanides and/or actinides are bound to the one or more proteins or devices, the one or more different lanthanides and/or actinides may be sequentially dissociated from the proteins. As an illustrative example, if both Nd and Dy are bound, one species of metal can be selectively dissociated, while the other metal remains bound. For example, one metal can be dissociated via contacting with a chelator, while the other metal is dissociated via adjustment of the pH. The one or more proteins and/or devices may be reused after the one or more lanthanides are unbound and separated.

A method of the present disclosure may be a method of detecting and/or quantifying the amount of one or more lanthanides and/or actinides in a sample. The method may comprise contacting the sample with one or more proteins and/or device of the present disclosure. The contacted sample may then be exposed to light and the resulting emission of the exposed contacted sample. The resulting emission results may then be compared to a known standard curve for a specific lanthanide or actinide. The concentration may then be determined by that comparison. Known standard curves may be prepared based on the desire to detect and/or determine the quantity of any specific lanthanide or actinide. Methods of preparing standard curves are known in the art.

The method of detecting and/or quantified may be performed on various samples. Non-limiting examples of samples include drinking water, wastewater, ground water, ash ponds, aqueous extract from contaminated soil, drainage (e.g., mine drainage, such as, for example, acidic mine drainage) or leachate (e.g., electronic waste leachate or leachate of an ore leachate). In various other examples, the sample is a solid sample. The method may be applied to samples over a variety of pH values. For example, the sample has a pH of 6 or below (e.g., 5.5 or below, 5 or below, 4.5 or below, 4 or below, 3.5 or below, or 3 or below). In various examples, the pH is greater than 6.

Various lanthanides (e.g., lanthanide ions) and/or actinides (e.g., actinide ions) may be bound by a protein and/or device. For example, the lanthanide is chosen from Tb, Eu, Dy, Sm, Nd, Yb, and ions thereof. In various examples, the lanthanide is Tb or an ion thereof. The bound lanthanides and/or actinides may be the same or different. The concentration of the lanthanide and/or actinide in the sample may be less than 1 ppm.

The following Statements provide various examples and embodiments of the present disclosure.
Statement 1. A protein capable of binding a metal and/or metal ion (e.g., in various examples, the protein is capable of dimerizing upon contact with a metal and/or metal ion), comprising a first EF hand motif, a second EF hand motif, a third EF hand motif, and a fourth EF hand motif, each EF hand motif comprising 11, 12, or 14 amino acid residues, wherein when the first EF hand motif, the second EF hand motif, the third EF hand motif, and the fourth EF hand motif have 12 amino acid residues, each EF hand motif has the following sequence:

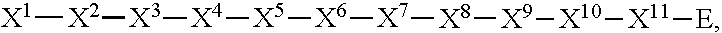

wherein
   i) for the first EF hand motif, the second EF hand motif, and the fourth EF hand motif:
      each X¹ is independently D or N;
      each X² is independently any canonical amino acid;
      each X³ is independently D, N, or E;
      each X⁴ is independently any canonical amino acid;
      each X⁵ is independently D, N, or E;
      each X⁶ is independently any canonical amino acid;
      each X⁷ is independently any canonical amino acid;
      each X⁸ is independently a hydrophobic residue;
      each X⁹ is independently a D, E, or T;
      each X¹⁰ is independently a hydrophobic residue; and
      each X¹¹ is independently any canonical amino acid;
   ii) for the third EF hand motif:
      X¹ is N;
      X² is any canonical amino acid;
      X³ is D;
      X⁴ is G or A;
      X⁵ is D or N;
      X⁶ is any canonical amino acid;
      X⁷ is T or S;
      X⁸ is a hydrophobic residue;
      X⁹ is E;
      X¹⁰ is a hydrophobic residue; and
      X¹¹ is D; and
   iii) the EF hand motifs are connected by 12 or 13 amino acid residue linkers and each amino acid residue of the linkers is a canonical amino acid, except for the third EF hand motif and the fourth EF hand motif, which are connect by the following sequence: (X)₅-R-(X)₆, wherein each X is independent a canonical amino acid and at least one amino acid of any of the linkers are hydrophobic.
Statement 2. A protein according to Statement 1, wherein X⁷ of the first EF hand motif, the second EF hand motif, and/or the fourth EF hand motif is independently T or S.
Statement 3. A protein according to Statement 1 or Statement 2, wherein X⁴ of the third EF hand motif is A.
Statement 4. A protein according to any one of the preceding Statements, wherein X⁸ of the third EF hand motif is L.
Statement 5. A protein according to any one of the preceding Statements, wherein X¹⁰ of the third EF hand motif is L, I, or M.
Statement 6. A protein having an enhanced REE/REE selectivity, comprising a first EF hand motif, a second EF hand motif, a third EF hand motif, and a fourth EF hand motif, each EF hand motif comprising 11, 12, or 14 amino acid residues, wherein when the first EF hand motif, the second EF hand motif, the third EF hand motif, and the fourth EF hand motif have 12 amino acid residues, each EF hand motif has the following sequence:

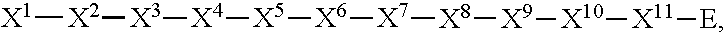

wherein,
   i) for the first EF hand motif and the fourth EF hand motif:
      each X¹ is independently D or N;
      each X² is independently any canonical amino acid;
      each X³ is independently D, N, or E;
      each X⁴ is independently any canonical amino acid;
      each X⁵ is independently D, N, or E;
      each X⁶ is independently any canonical amino acid;
      each X⁷ is independently any canonical amino acid;
      each X⁸ is independently a hydrophobic residue;
      each X⁹ is independently a D, E, or T;
      each X¹⁰ is independently a hydrophobic residue; and
      each X¹¹ is independently any canonical amino acid;
   ii) for the second EF hand motif:
      X¹ is N;
      X² is any canonical amino acid;
      X³ is D;
      X⁴ is any canonical amino acid;
      X⁵ is D;
      X⁶ is any canonical amino acid;
      X⁷ is T or S;
      X⁸ is a hydrophobic residue;
      X⁹ is E;
      X¹⁰ is any canonical amino acid; and
      X¹¹ is any canonical amino acid; and
   iii) for the third EF hand motif:
      X¹ is D;
      X² is any canonical amino acid;
      X³ is D;
      X⁴ is D;
      X⁵ is D;
      X⁶ is G;
      X⁷ is T or S;
      X⁸ is a hydrophobic residue;
      X⁹ is D;
      X¹⁰ is any canonical amino acid; and
      X¹¹ is any canonical amino acid;
   iv) at least one X² of the second EF hand motif and third EF hand motif is P; and
   v) the EF hand motifs are connected by 12 or 13 amino acid residue linkers, wherein each amino acid of the linkers is a canonical amino acid and at least one amino acid of any of the linkers is hydrophobic.
Statement 7. A protein according to Statement 6, wherein X⁸ of the second EF hand motif and/or third EF hand motif is L, I, M, or V.
Statement 8. A protein according to any one of the preceding Statements, wherein the protein comprises the sequence:

(SEQ ID NO: 1)
MKLSLKAGAA ITAFVFAASP VLAASGADAL KALNKDNDDS
LEIAEVIHAG ATTFTAINPD GDTTLESGET KGRLTEKDWA
RANKDGDQTL EMDEWLKILR TRFKRADANK DGKLTAAELD
SKAGQGVLVM IMK
or
(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK

or a protein having 70%, 75%, 80%, 85%, 90%, 91%, 92%, 93%, 94%, 95%, 96%, 97%, 98%, or 99% identity to either SEQ ID NO:1 or SEQ ID NO:2 or comprises the following sequence:

(SEQ ID NO: 7)
MLTGKEFLRKYNKDKDSTVEIVEAIDLGTKVFKAINPDKDKTLEAAETK
GRLSDEDWAQFNKDGDKTLELDEWLIIVRKRFNDADANKDGKLTEAELD
APAGQQLILLIAK,

or a protein having 70% identity thereto.
Statement 9. A protein according to any one of the preceding Statements, wherein the protein is complexed with a rare earth element.
Statement 10. A protein according to Statement 9, wherein the rare earth element is a light rare earth element.
Statement 11. A protein according to Statement 9, wherein the rare earth element is a heavy rare earth element.
Statement 12. A protein according to any one of the preceding Statements, wherein the protein comprises the following sequence:

(SEQ ID NO: 44)
MASGADAL KALNKDNDDS LEIAEVIHAG ATTFTAINPD
GDTTLESGET KGRLTEKDWA RANKDGDQTL EMDEWLKILX
TRFKRADANK DGKLTAAELD SKAGQGVLVM IMK,

where X is any canonical amino acid residue other than R.
Statement 13. A protein according to Statement 12, wherein the protein comprises the following sequence:

(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK

Statement 14. A protein according to any one of Statements 1-12, wherein the protein has the following sequence:

>Hans-LanM (full protein sequence, signal peptide underlined)
(SEQ ID NO: 1)
MKLSLKAGAA ITAFVFAASP VLAASGADAL KALNKDNDDS LEIAEVIHAG
ATTFTAINPD GDTTLESGET KGRLTEKDWA RANKDGDQTL EMDEWLKILR
TRFKRADANK DGKLTAAELD SKAGQGVLVM IMK
>Hans-LanM (with signal peptide removed, as expressed herein)
(SEQ ID NO: 2)
MASGADAL KALNKDNDDS LEIAEVIHAG ATTFTAINPD GDTTLESGET
KGRLTEKDWA RANKDGDQTL EMDEWLKILR TRFKRADANK DGKLTAAELD
SKAGQGVLVM IMK
>Hans-LanM(R100K) (with signal peptide removed, substitution underlined)
(SEQ ID NO: 4)
MASGADAL KALNKDNDDS LEIAEVIHAG ATTFTAINPD GDTTLESGET
KGRLTEKDWA RANKDGDQTL EMDEWLKILK TRFKRADANK DGKLTAAELD
SKAGQGVLVM IMK
>Hans-LanM-Cys (for immobilization)
(SEQ ID NO: 5)
MASGADAL KALNKDNDDS LEIAEVIHAG ATTFTAINPD GDTTLESGET
KGRLTEKDWA RANKDGDQTL EMDEWLKILR TRFKRADANK DGKLTAAELD
SKAGQGVLVM IMKGSGC
>Hans-LanM(R100K)-Cys (for immobilization)
(SEQ ID NO: 6)
MASGADAL KALNKDNDDS LEIAEVIHAG ATTFTAINPD GDTTLESGET
KGRLTEKDWA RANKDGDQTL EMDEWLKILK TRFKRADANK DGKLTAAELD
SKAGQGVLVM IMKGSGC
>LanM_012 (Xanthobacter flavus, with signal peptide removed)
(SEQ ID NO: 7)
MLTGKEFLRKYNKDKDSTVEIVEAIDLGTKVFKAINPDKDKTLEAAETKGRLSDED
WAQFNKDGDKTLELDEWLIIVRKRFNDADANKDGKLTEAELDAPAGQQLILLIAK
>LanM_013 (expressed construct; signal peptide removed and N-terminal Met added, EF
hands underlined):
(SEQ ID NO: 8)
MGKAADAIQALDPDKDGTIDLNEAKAGAKAVFEKINPDGDGTLEVKELKGRLTKKE
LDAADPDNDGTLDMQEYEAVVTKQFELANPDNDGTVDEKELKTKEGKKLLKLIY
>LanM_013 (full-length sequence from Methyloligella sp. GL2, with signal peptide,
underlined):
(SEQ ID NO: 9)
MAAILTIAGAVTVAAGGAAFAGKAADAIQALDPDKDGTIDLNEAKAGAKAVFEKIN
PDGDGTLEVKELKGRLTKKELDAADPDNDGTLDMQEYEAVVTKQFELANPDNDGT
VDEKELKTKEGKKLLKLIY
>Methyloligella halotolerans (expected to behave similarly to LanM_013; sequence with
signal peptide removed)
(SEQ ID NO: 10)
MADAEISDTMKVVDPDMDNALTLEEAQAAGAKVFKKLNTDDDNTLEADELKGRVS
ERQLKKADPDDDGSLDMAEYEALIKKRFEAANPDGDDTIESDELETKKGKKLLELIQE
>Mex-LanM-A32D/A117K (mutations underlined)
(SEQ ID NO: 11)
MAPTTTTKVDIDAFDPDKDGTIDLKEALAAGSAAFDKLDPDKDGTLDAKELKGRVS
EADLKKLDPDNDGTLDKKEYLAAVEAQFKAANPDNDGTIDKRELASPAGSALVNLIR
>Mex-LanM-A32D/A117R (mutations underlined)
(SEQ ID NO: 12)
MAPTTTTKVDIDAFDPDKDGTIDLKEALAAGSAAFDKLDPDKDGTLDAKELKGRVS
EADLKKLDPDNDGTLDKKEYLAAVEAQFKAANPDNDGTIDRRELASPAGSALVNLIR
>Mex-LanM-I42L/N108D/1115L (mutations underlined)
(SEQ ID NO: 13)
MAPTTTTKVDIAAFDPDKDGTLDLKEALAAGSAAFDKLDPDKDGTLDAKELKGRVS
EADLKKLDPDNDGTLDKKEYLAAVEAQFKAADPDNDGTLDARELASPAGSALVNLIR
>LanM_011 (Hyphomicrobium, with signal peptide removed)
(SEQ ID NO: 14)
MGHHNCKAEMAYLNPDHDGTIDWREARRAAVRLFHKLDPDHDGTLDMKEVRGRV
GILSFARFNPDRDGKLDKHEWLALVKHRFHRANPDKDGTIDCRELHSLAGRKLLRVLM
>Unclassified Hyphomicrobium (signal peptide removed; appears to only have 3 functional
EF hands)
(SEQ ID NO: 15)
MGHRSAKAHPSCPALNAIDPDGDGAMTLGEAKRAAIKTFMKLNKDGDITLELDELG
GRMSAAAFAQADLIKGRGISLGEYLIEVRRRFKWANPDKDHTIECDELHSKYGRLLA
RLLK
>HansR100K-L1
(SEQ ID NO: 16)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSASGADALKALNKDNDDSLEIAEVIHAGATTFTAINPDGD
TTLESGETKGRLTEKDWARANKDGDQTLEMDEWLKILKTRFKRADANKDGKLTAA
ELDSKAGQGVLVMIMK
>HansR100K-L2
(SEQ ID NO: 17)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSASGADALKALNKDNDDSLEIAEVIHA
GATTFTAINPDGDTTLESGETKGRLTEKDWARANKDGDQTLEMDEWLKILKTRFKR
ADANKDGKLTAAELDSKAGQGVLVMIMK
>HansR100K-L3
(SEQ ID NO: 18)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSGGSGGSASGADALKALNKDNDDSLEI
AEVIHAGATTFTAINPDGDTTLESGETKGRLTEKDWARANKDGDQTLEMDEWLKIL
KTRFKRADANKDGKLTAAELDSKAGQGVLVMIMK
>HansR100K-L4
(SEQ ID NO: 19)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSGGSGGSGGSGGSASGADALKALNKD
NDDSLEIAEVIHAGATTFTAINPDGDTTLESGETKGRLTEKDWARANKDGDQTLEMD
EWLKILKTRFKRADANKDGKLTAAELDSKAGQGVLVMIMK
>HansR100K-L5
(SEQ ID NO: 20)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GSGGSGAEAAAKEAAAKAGGSGGSAEAAAKEAAAKAGSGGSGASGADALKALNK
DNDDSLEIAEVIHAGATTFTAINPDGDTTLESGETKGRLTEKDWARANKDGDQTLEM
DEWLKILKTRFKRADANKDGKLTAAELDSKAGQGVLVMIMK
>Mex-LanM-G51A (mutations underlined)
(SEQ ID NO: 21)
MAPTTTTKVDIAAFDPDKDGTIDLKEALAAASAAFDKLDPDKDGTLDAKELKGRVS
EADLKKLDPDNDGTLDKKEYLAAVEAQFKAANPDNDGTIDARELASPAGSALVNLIR
>Mex-LanM-A98G (mutations underlined)
(SEQ ID NO: 22)
MAPTTTTKVDIAAFDPDKDGTIDLKEALAAGSAAFDKLDPDKDGTLDAKELKGRVS
EADLKKLDPDNDGTLDKKEYLGAVEAQFKAANPDNDGTIDARELASPAGSALVNLIR
>Mex-LanM-A99G (mutations underlined)
(SEQ ID NO: 23)
MAPTTTTKVDIAAFDPDKDGTIDLKEALAAGSAAFDKLDPDKDGTLDAKELKGRVS
EADLKKLDPDNDGTLDKKEYLAGVEAQFKAANPDNDGTIDARELASPAGSALVNLIR
>Mex-LanM-V100G (mutations underlined)
(SEQ ID NO: 24)
MAPTTTTKVDIAAFDPDKDGTIDLKEALAAGSAAFDKLDPDKDGTLDAKELKGRVS
EADLKKLDPDNDGTLDKKEYLAAGEAQFKAANPDNDGTIDARELASPAGSALVNLIR
>Mex-LanM-A102G (mutations underlined)
(SEQ ID NO: 25)
MAPTTTTKVDIAAFDPDKDGTIDLKEALAAGSAAFDKLDPDKDGTLDAKELKGRVS
EADLKKLDPDNDGTLDKKEYLAAVEGQFKAANPDNDGTIDARELASPAGSALVNLIR
>Hans-LanM-I43A (mutation underlined)
(SEQ ID NO: 26)
MASGADALKALNKDNDDSLEAAEVIHAGATTFTAINPDGDTTLESGETKGRLTEKD
WARANKDGDQTLEMDEWLKILRTRFKRADANKDGKLTAAELDSKAGQGVLVMIMR
>Hans-LanM-I43V (mutation underlined)
(SEQ ID NO: 27)
MASGADALKALNKDNDDSLEVAEVIHAGATTFTAINPDGDTTLESGETKGRLTEKD
WARANKDGDQTLEMDEWLKILRTRFKRADANKDGKLTAAELDSKAGQGVLVMIMK
>Hans-LanM-A44N (mutation underlined)
(SEQ ID NO: 28)
MASGADALKALNKDNDDSLEINEVIHAGATTFTAINPDGDTTLESGETKGRLTEKDW
ARANKDGDQTLEMDEWLKILRTRFKRADANKDGKLTAAELDSKAGQGVL VMIMK
>Hans-LanM-A44S (mutation underlined)
(SEQ ID NO: 29)
MASGADALKALNKDNDDSLEISEVIHAGATTFTAINPDGDTTLESGETKGRLTEKDW
ARANKDGDQTLEMDEWLKILRTRFKRADANKDGKLTAAELDSKAGQGVLVMIMK
>Hans-LanM-A44T (mutation underlined)
(SEQ ID NO: 30)
MASGADALKALNKDNDDSLEITEVIHAGATTFTAINPDGDTTLESGETKGRLTEKDW
ARANKDGDQTLEMDEWLKILRTRFKRADANKDGKLTAAELDSKAGQGVLVMIMK
>Hans-LanM-I47A (mutation underlined)
(SEQ ID NO: 31)
MASGADALKALNKDNDDSLEIAEVAHAGATTFTAINPDGDTTLESGETKGRLTEKD
WARANKDGDQTLEMDEWLKILRTRFKRADANKDGKLTAAELDSKAGQGVLVMIMK
>Hans-LanM-I47V (mutation underlined)
(SEQ ID NO: 32)
MASGADALKALNKDNDDSLEIAEVVHAGATTFTAINPDGDTTLESGETKGRLTEKD
WARANKDGDQTLEMDEWLKILRTRFKRADANKDGKLTAAELDSKAGQGVLVMIMK
>Hans-LanM-M92L (mutation underlined)
(SEQ ID NO: 33)
MASGADALKALNKDNDDSLEIAEVIHAGATTFTAINPDGDTTLESGETKGRLTEKDW
ARANKDGDQTLELDEWLKILRTRFKRADANKDGKLTAAELDSKAGQGVLVMIMK
>Hans-LanM-M92A (mutation underlined)
(SEQ ID NO: 34)
MASGADALKALNKDNDDSLEIAEVIHAGATTFTAINPDGDTTLESGETKGRLTEKDW
ARANKDGDQTLEADEWLKILRTRFKRADANKDGKLTAAELDSKAGQGVLVMIMK
>Hans-LanM-M92D (mutation underlined)
(SEQ ID NO: 35)
MASGADALKALNKDNDDSLEIAEVIHAGATTFTAINPDGDTTLESGETKGRLTEKDW
ARANKDGDQTLEDDEWLKILRTRFKRADANKDGKLTAAELDSKAGQGVLVMIMK
>Hans-LanM-D93A (mutation underlined)
(SEQ ID NO: 36)
MASGADALKALNKDNDDSLEIAEVIHAGATTFTAINPDGDTTLESGETKGRLTEKDW
ARANKDGDQTLEMAEWLKILRTRFKRADANKDGKLTAAELDSKAGQGVLVMIMK
>Hans-LanM-D93N (mutation underlined)
(SEQ ID NO: 37)
MASGADALKALNKDNDDSLEIAEVIHAGATTFTAINPDGDTTLESGETKGRLTEKDW
ARANKDGDQTLEMNEWLKILRTRFKRADANKDGKLTAAELDSKAGQGVLVMIMK
>HansR100K-L1
(SEQ ID NO: 16)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSASGADALKALNKDNDDSLEIAEVIHAGATTFTAINPDGD
TTLESGETKGRLTEKDWARANKDGDQTLEMDEWLKILKTRFKRADANKDGKLTAA
ELDSKAGQGVLVMIMK
>HansR100K-L2
(SEQ ID NO: 17)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSASGADALKALNKDNDDSLEIAEVIHA
GATTFTAINPDGDTTLESGETKGRLTEKDWARANKDGDQTLEMDEWLKILKTRFKR
ADANKDGKLTAAELDSKAGQGVLVMIMK
>HansR100K-L3
(SEQ ID NO: 18)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSGGSGGSASGADALKALNKDNDDSLEI
AEVIHAGATTFTAINPDGDTTLESGETKGRLTEKDWARANKDGDQTLEMDEWLKIL
KTRFKRADANKDGKLTAAELDSKAGQGVLVMIMK
>HansR100K-L4
(SEQ ID NO: 19)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GGSGGSGGSGGSGGSGGSGGSGGSGGSGGSGGSGGSGGSGGSASGADALKALNKD
NDDSLEIAEVIHAGATTFTAINPDGDTTLESGETKGRLTEKDWARANKDGDQTLEMD
EWLKILKTRFKRADANKDGKLTAAELDSKAGQGVLVMIMK
>HansR100K-L5
(SEQ ID NO: 20)
MASGADALKALNKDNDDSLEIAEVIHAGATTFTAINPDGDTTLESGETKGRLTEKDW
ARANKDGDQTLEMDEWLKILKTRFKRADANKDGKLTAAELDSKAGQGVLVMIMK
GSGGSGAEAAAKEAAAKAGGSGGSAEAAAKEAAAKAGSGGSGASGADALKALNK
DNDDSLEIAEVIHAGATTFTAINPDGDTTLESGETKGRLTEKDWARANKDGDQTLEM
DEWLKILKTRFKRADANKDGKLTAAELDSKAGQGVLVMIMK
>Hans-LanM(3E9Q)
(SEQ ID NO: 38)
MASGADAL KALNKDNDDS LQIAEVIHAG ATTFTAINPD GDTTLQSGET
KGRLTEKDWA RANKDGDQTL QMDEWLKILR TRFKRADANK DGKLTAAELD
SKAGQGVLVM IMK
>Mex-LanM-N108D/A124G (mutations underlined)
(SEQ ID NO: 39)
MAPTTTTKVDIAAFDPDKDGTIDLKEALAAGSAAFDKLDPDKDGTLDAKELKGRVS
EADLKKLDPDNDGTLDKKEYLAAVEAQFKAADPDNDGTIDARELASPGGSALVNLIR
>Mex-LanM-N108D/A127G (mutations underlined)
(SEQ ID NO: 40)
MAPTTTTKVDIAAFDPDKDGTIDLKEALAAGSAAFDKLDPDKDGTLDAKELKGRVS
EADLKKLDPDNDGTLDKKEYLAAVEAQFKAADPDNDGTIDARELASPAGSGLVNLIR
>Mex-LanM-N108D/A102G (mutations underlined)
(SEQ ID NO: 41)
MAPTTTTKVDIAAFDPDKDGTIDLKEALAAGSAAFDKLDPDKDGTLDAKELKGRVS
EADLKKLDPDNDGTLDKKEYLAAVEGQFKAADPDNDGTIDARELASPAGSALVNLIR;
>Mex-LanM-N108D
(SEQ ID NO: 42)
MAPTTTTKVDIAAFDPDKDGTIDLKEALAAGSAAFDKLDPDKDGTLDAKELKGRVS
EADLKKLDPDNDGTLDKKEYLAAVEAQFKAADPDNDGTIDARELASPAGSALVNLIR.

Statement 15. A protein according to any one of Statements 1-12, wherein the protein has the following sequence:

(SEQ ID NO: 1)
MKLSLKAGAA ITAFVFAASP VLAASGADAL KALNKDNDDS
LEIAEVIHAG ATTFTAINPD GDTTLESGET KGRLTEKDWA
RANKDGDQTL EMDEWLKILR TRFKRADANK DGKLTAAELD
SKAGQGVLVM IMK;
(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK;
or
(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK.

Statement 16. A device comprising a protein according to any one of the preceding Statements.
Statement 17. A device according to Statement 16, wherein the device is a filter, membrane, sensor, handheld detector, plate reader, fluorometer, biosensor, or in-line monitor.
Statement 18. A kit comprising a protein according to any one of Statements 1 to 15 or a device comprising a protein according to any one of Statements 1 to 15.
Statement 19. A method of isolating a rare earth element comprising contacting a protein according to any one of Statements 1 to 15 with a sample comprising a rare earth element, wherein the rare earth element binds to one or more proteins according to any one of Statements 1 to 15, and removing the protein from the sample.
Statement 20. A method according to Statement 19, wherein the sample is drinking water, wastewater, ground water, ash ponds, aqueous extract from contaminated soil, drainage, leachate, aqueous extract or leachate from a solid waste such as electronic waste or from an ore or mine tailings, or solid sample.
Statement 21. A method according to Statements 19 or 20, wherein the one or more rare earth element is a lanthanide are chosen from La, Ce, Pr, Nd, Pm, Sm, Eu, Gd, Tb, Dy, Ho, Er, Tm, Yb, Lu, Sc, Y, and ions thereof.
Statement 22. A method according to any one of Statements 19 to 21, wherein the method further comprises detecting and quantifying the one or more rare earth element. Statement 23. A method according to any one of Statements 19 to 22, wherein a plurality of different rare earth elements are bound to the protein.
Statement 24. A method according to Statement 23, wherein each different rare earth element is separated individually from the protein.
Statement 25. A method of determining if a light rare earth element is present in a sample, comprising contacting the sample with a protein according to any one of Statements 1 to 15, and determining if the protein has formed a dimer.

The following examples provide various examples of the present disclosure. They are not intended to be limiting in any manner.

### Example 1

This Example describes use of the peptides/proteins of the present disclosure.

Technologically critical rare-earth elements are notoriously difficult to separate, owing to their subtle differences in ionic radius and coordination number. The natural lanthanide-binding protein, lanmodulin, is a sustainable alternative to conventional solvent extraction-based separation. Characterized herein is a novel lanmodulin, from Hansschlegelia quercus (Hans-LanM), with an oligomeric state sensitive to rare-earth ionic radius, the lanthanum(III)-induced dimer being >100-fold tighter than the dysprosium(III)-induced dimer. X-ray crystal structures illustrate how picometer-scale differences in radius between lanthanum(III) and dysprosium(III) are propagated to Hans-LanM's quaternary structure via a carboxylate shift that rearranges a second-sphere hydrogen-bonding network. Comparison to the prototypal lanmodulin from Methylorubrum extorquens reveals distinct metal coordination strategies, rationalizing Hans-LanM's greater selectivity within the rare earths. Finally, structure-guided mutagenesis of a key residue at the Hans-LanM dimer interface modulates dimerization in solution and enables single-stage, column-based separation of a neodymium(III)/dysprosium(III) mixture to >98% individual element purities. The present disclosure showcases the natural diversity of selective lanthanide recognition motifs, and it reveals rare-earth-sensitive dimerization as a biological principle by which to tune the performance of biomolecule-based separation processes.

Described herein is a lanmodulin from Hansschlegelia quercus (Hans-LanM), a methylotrophic bacterium isolated from English oak buds, exhibits enhanced RE separation capacity relative to Mex-LanM. Whereas Mex-LanM is always monomeric, Hans-LanM exists in a monomer/dimer equilibrium, the position of which depends on the specific RE bound. Three X-ray crystal structures of lanmodulins and structure-guided mutagenesis explain Hans-LanM's RE-dependent oligomeric state and its greater separation capacity than Mex-LanM's. Also described is a single-stage Hans-LanM-based separation of the critical neodymium/dysprosium pair. These results illustrate how intermolecular interactions—common in proteins but rare in small molecules—may be exploited to improve RE separations.

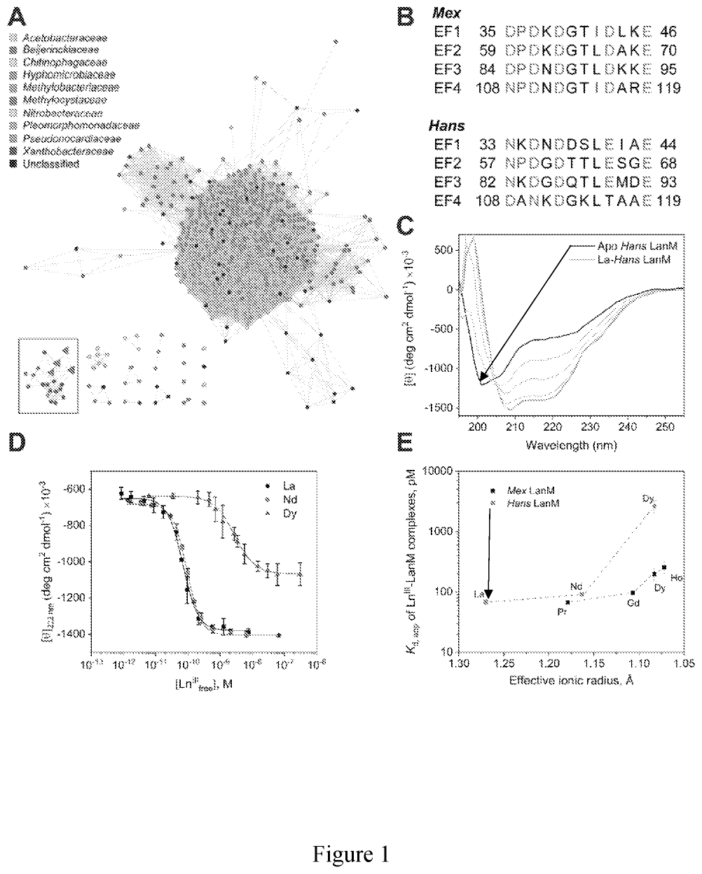

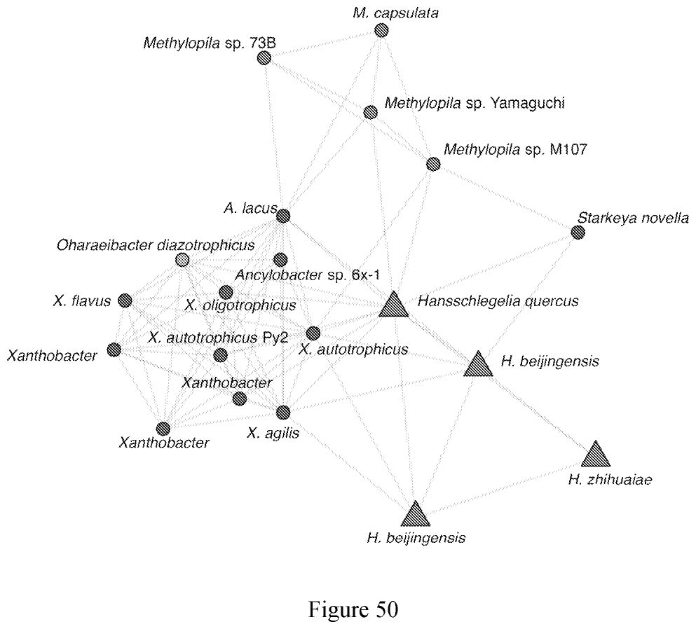

Hans-LanM's distinct selectivity profile. Several hallmarks of a lanmodulin have been proposed. First, LanM proteins possess four EF-hand motifs. EF-hands comprise 12-residue, carboxylate-rich metal-binding loops flanked by α-helices, which traditionally respond to Ca^{II} binding; in Mex-LanM, however, EF-hands 1-3 bind lanthanide(III) ions with low-picomolar affinity and 10⁸-fold selectivity over Ca^{II}, resulting in a large, lanthanide-selective disorder-to-order conformational transition. EF4 binds with only micromolar affinity. Second, adjacent EF-hands in LanMs are separated by 12-13 residues—rather than the typical ˜25 residues in Ca^{II}-responsive EF-hand proteins—resulting in an unusual three-helix bundle architecture with the metal-binding sites on the periphery. Third, at least one EF-hand contains proline at the 2^{nd} position (in Mex-LanM, all four EF-hands feature P₂ residues). Databases were searched using the first two criteria and a sequence length of <200 residues, identifying 696 putative LanMs. These sequences were visualized using a sequence similarity network to identify LanM sequences that cluster separately from Mex-LanM. At a 65% identity threshold, a small cluster of sequences that is remote from the main cluster of 642 sequences is formed (FIG. 1a). This exclusive cluster (the “Hans cluster”), includes bacteria from several genera, including Hansschlegelia and Xanthobacter (FIG. 50), all of which are facultative methylotrophs.

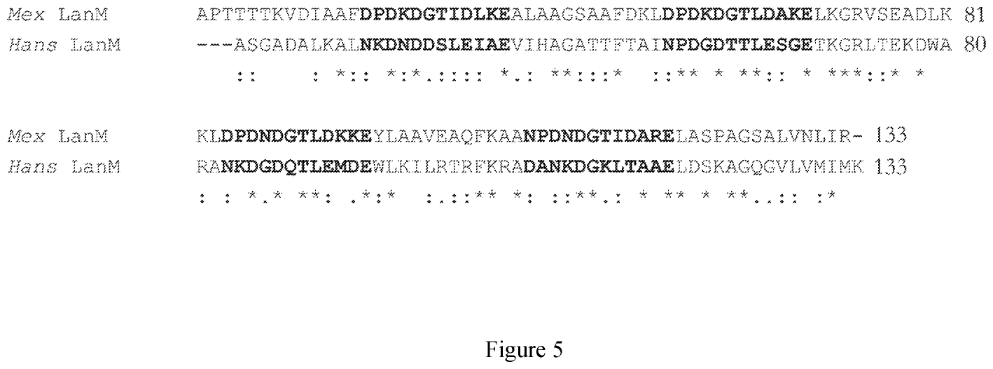

Hans-LanM features low (33%) sequence identity with Mex-LanM (FIG. 5) and divergent EF-hand motifs, particularly at the first, second, and ninth positions (FIG. 1b), which are important positions in Mex-LanM and other EF-hand proteins. Therefore, Hans-LanM presented an opportunity to determine features essential for selective lanthanide recognition in lanmodulins.

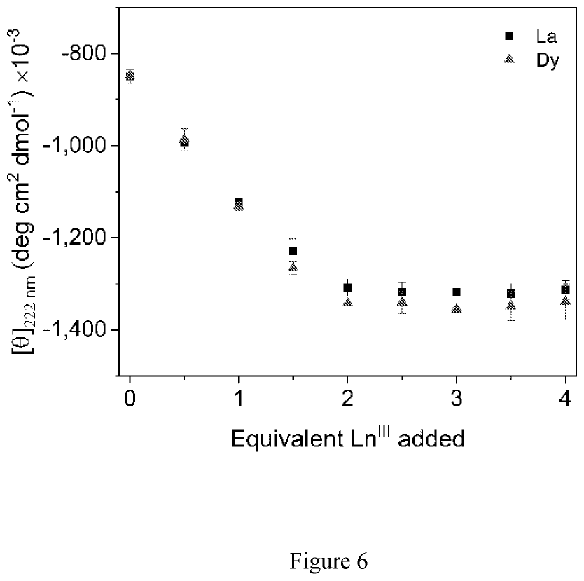

Hans-LanM was expressed in E. coli as a 110 amino-acid protein (FIG. 5). La^{III} and Nd^{III} were selected as representative LREs and Dy^{III} as representative HRE for complexation studies. The protein binds ˜3 equivalents La^{III} and Nd^{III}, and slightly less Dy^{III}, by ICP-MS (Table 1), as does Mex-LanM. Also like Mex-LanM, Hans-LanM exhibits little helical content in the absence of metal, as judged by the circular dichroism (CD) signal at 222 nm (FIG. 1c). Unexpectedly, only 2 equivalents of La^{III} or Dy^{III} were sufficient to cause Hans-LanM's complete conformational change (FIG. 6), indicating that the third binding equivalent is weak and does not increase helicity.

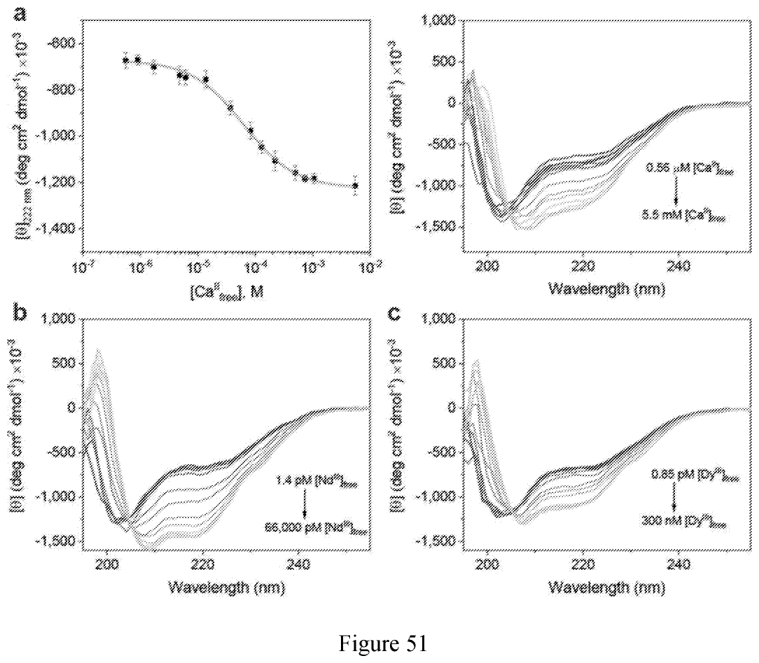

The apparent dissociation constants (K_{d,app}) determined by CD spectroscopy reflect the RE/RE and RE/non-RE selectivities of Mex-LanM under competitive RE recovery conditions. Therefore, similar determinations of K_{d,app} with free metal concentrations controlled by a competitive chelator were applied to Hans-LanM; the results (FIG. 1d, Table 2) diverged from Mex-LanM. Binding of La^{III} and Nd^{III} to Hans-LanM increases molar ellipticity at 222 nm by 2.3-fold, the full conformational change evident in stoichiometric titrations. The conformational change is cooperative (Hill coefficients, n, of 2, Table 2), and the K_{d,app} values are similar, 68 and 91 μM respectively. By contrast, even though Dy^{III} induces the same overall response as La^{III} in stoichiometric titrations (FIG. 6), in the chelator-buffered Dy^{III} titrations Hans-LanM exhibits a lesser conformational response (1.8-fold increase). This difference indicates that at least one of the Dy^{III} binding sites is very weakly responsive (K_{d,app}>0.3 μM, the highest concentration accessible in the chelator-buffered titrations). The main response to Dy^{III} occurs at 2.6 nM, >30-fold higher than with the LREs, and with little or no cooperativity (n=1.3). By contrast, Mex-LanM shows only a modest preference for LREs (˜5-fold, FIG. 1e), and all lanthanides and Y^{III} induce similar conformational changes and cooperativity. Hans-LanM responds to calcium(II) weakly (K_{d,app}=60 μM), with the same lack of cooperativity (n=1.0) and partial conformational change evident with Dy^{III} (FIG. 51). Therefore, Hans-LanM discriminates more strongly between LREs and HREs than does Mex-LanM, with the HRE complexes exhibiting lower affinity, lesser cooperativity, and a lesser primary conformational change.

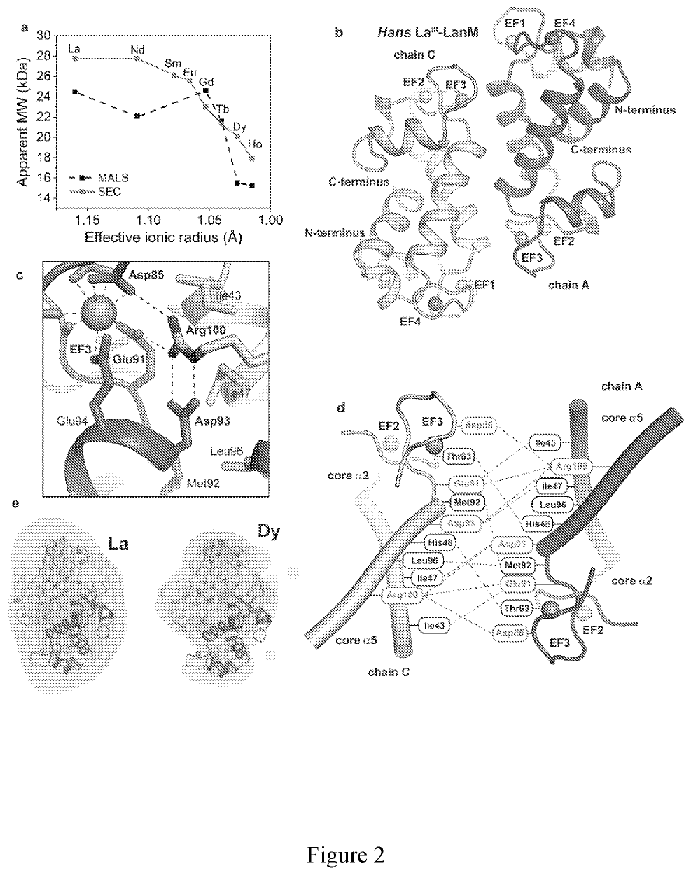

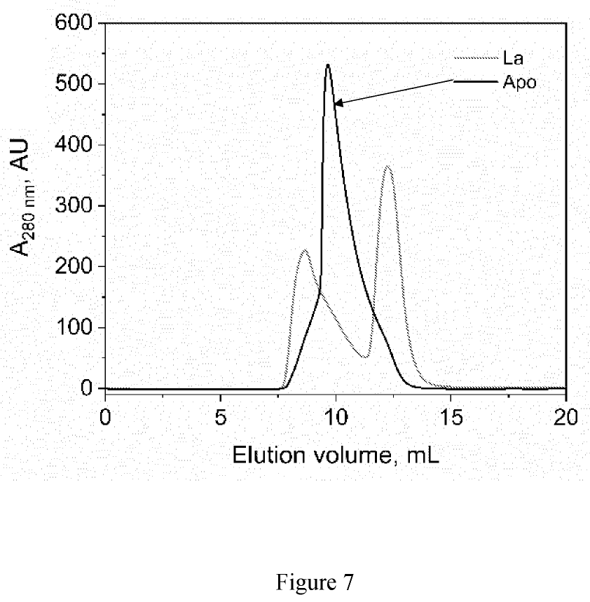

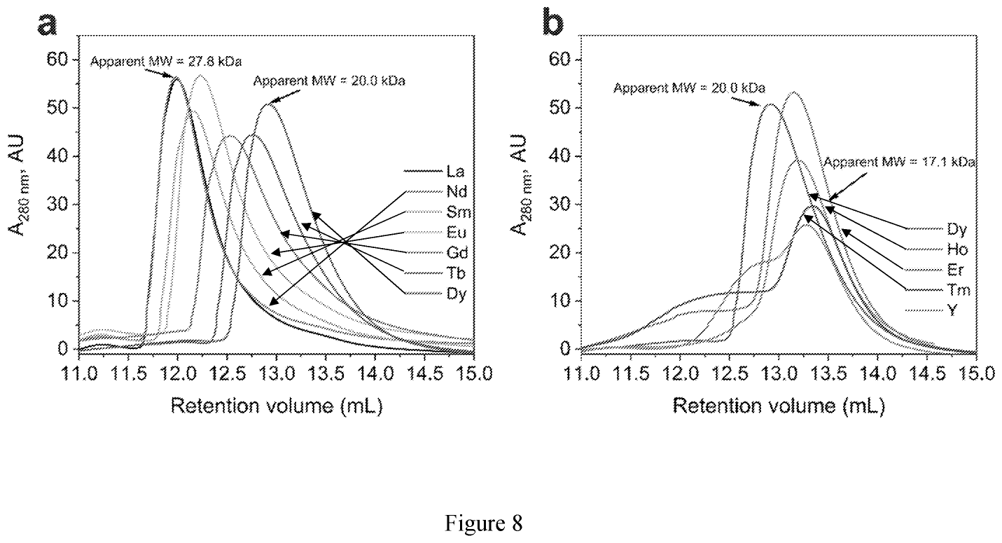

LRE-selective dimerization. The distinct behaviors of the LRE- and HRE-Hans-LanM complexes suggested mechanism(s) of LRE/HRE selectivity not present in Mex-LanM. Because Mex-LanM is a monomer in complex with LREs and HREs alike, it was considered that LREs and HREs might induce different oligomeric states in Hans-LanM. In the presence of 3 equiv. La^{III}, Hans-LanM elutes from a size-exclusion chromatography (SEC) column not at the expected molecular weight (MW) of 11.9 kDa but instead at 27.8 kDa, suggestive of a dimer (FIG. 7, FIG. 8a). Starting gradually after Nd^{III} but sharply at Gd^{III}, the apparent MW decreases towards that expected for a monomer (FIG. 2a, FIG. 8, Table 3). Notably, lanthanides heavier than Gd^{III} do not appear to support growth of RE-utilizing bacteria.

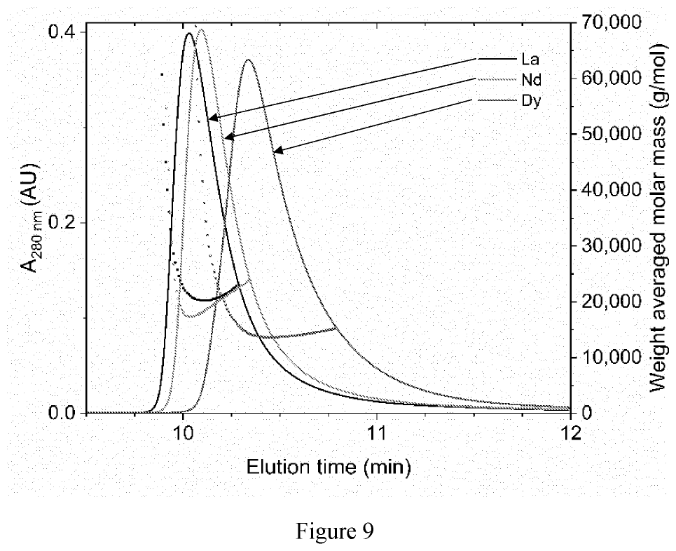

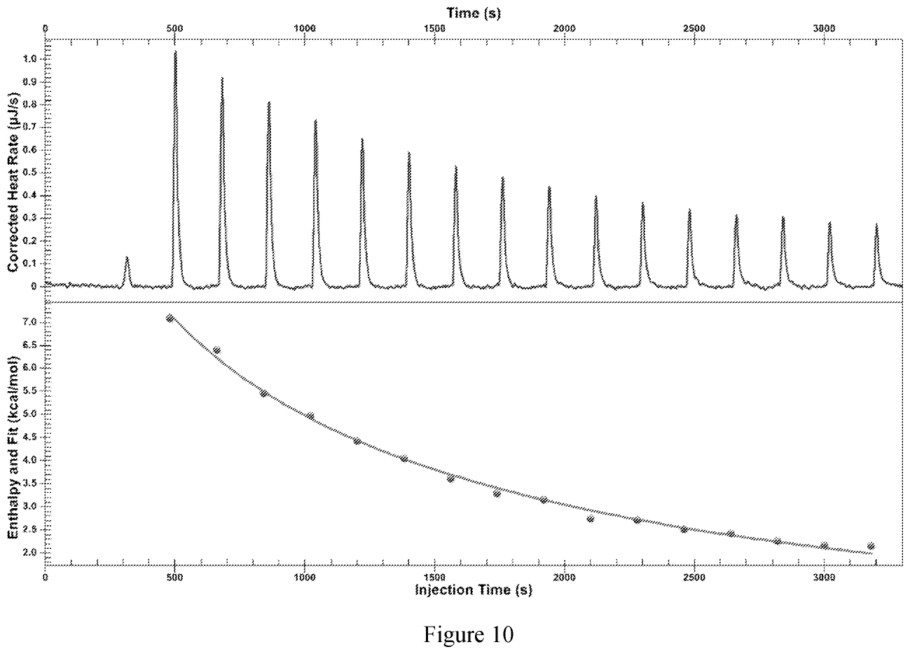

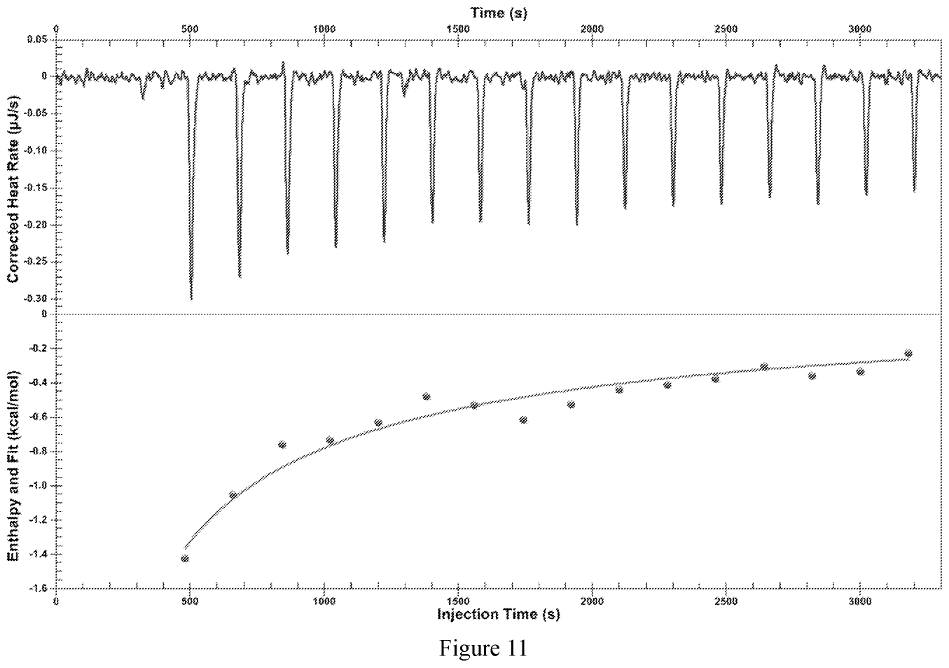

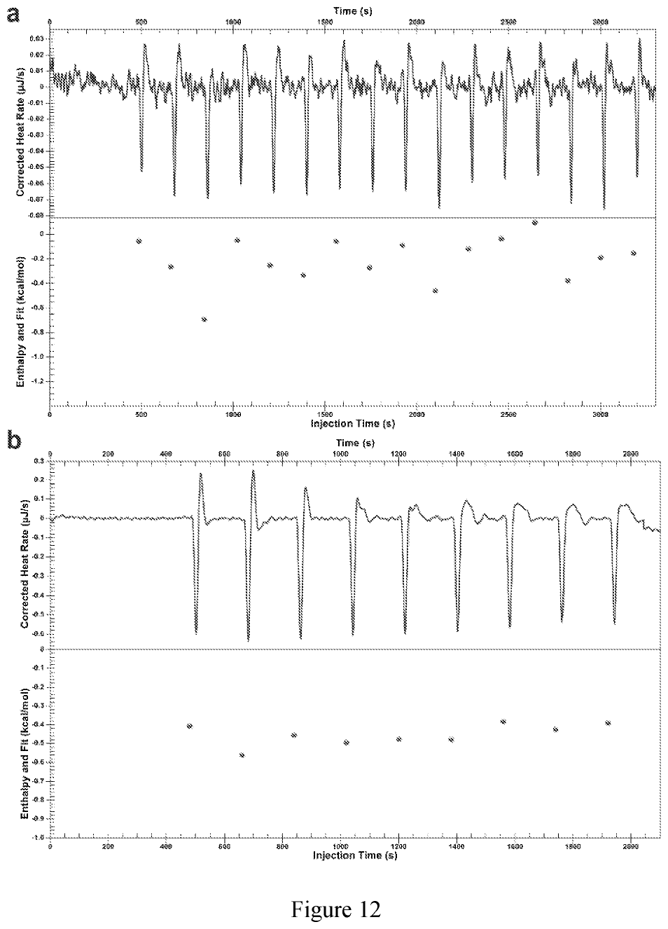

To provide further support for preferential dimerization in the presence of physiologically relevant LREs, RE complexes of Hans-LanM were analyzed using multi-angle light scattering (MALS) (FIG. 2a, FIG. 9). The La^{III}, Nd^{III}, and Gd^{III} complexes have MWs of 22-25 kDa, indicative of dimers, but MWs decrease starting with Tb^{III} and continue to Dy^{III} and Ho^{III}, at ˜15 kDa (Table 17), in agreement with the SEC data. Ca^{II}-bound Hans-LanM also indicated a MW of 14.7 kDa. The HRE-, Ca^{II}-, and apo-Hans-LanM complexes are still one-third larger than expected for a monomer, however, suggesting that these forms exist in a rapid equilibrium with ˜2:1 monomer:dimer ratio under these conditions. The K_{d} for dimerization (K_{dimer}) of apo-, La^{III}-bound, and Dy^{III}-bound Hans-LanM was determined by isothermal titration calorimetry (ITC) (FIG. 10-12, Table 18). The apoprotein and Dy^{III}-bound protein weakly dimerize, with K_{dimer}s of 117 μM and 60 μM, respectively, consistent with the ratios of monomer and dimer reflected in the SEC and MALS traces. In the presence of La^{III}, however, the dimer was too tight to be able to observe monomerization by ITC, which indicates that K_{dimer}<0.4 μM (FIG. 12). Thus, La^{III} favors Hans-LanM's dimerization by >100-fold over Dy^{III}.

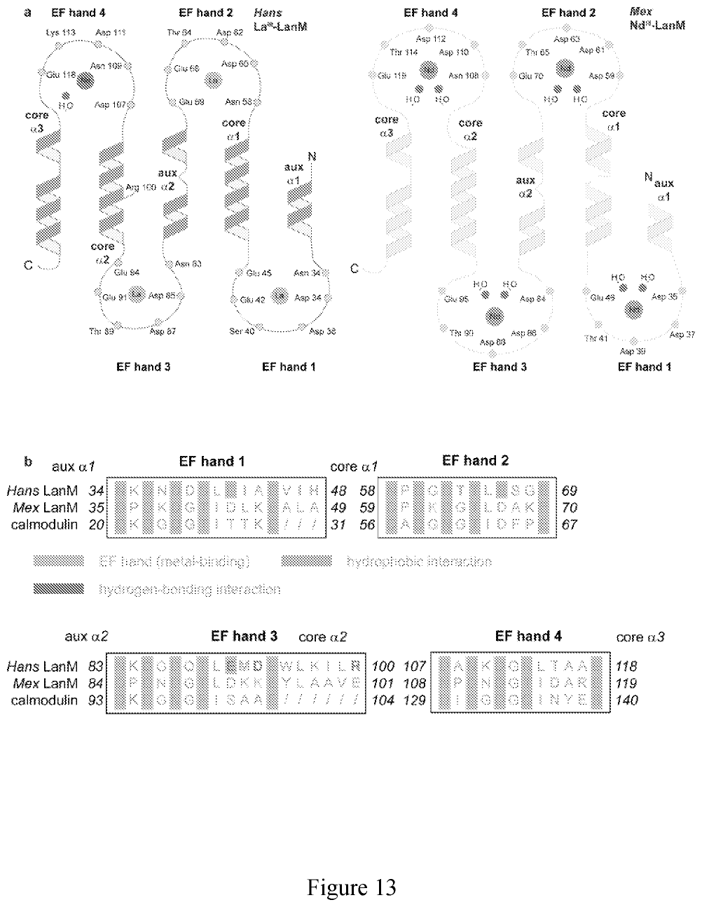

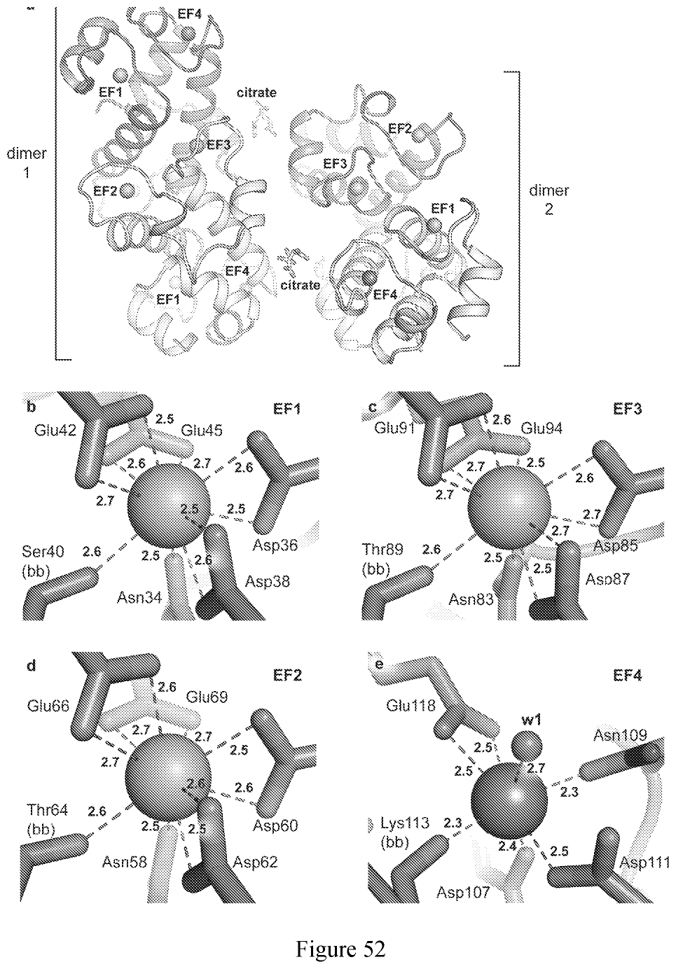

A 1.8-Å-resolution X-ray crystal structure of Hans-LanM in complex with La^{III} confirms LRE-induced dimerization (FIG. 52, Table 4). Two LanM monomers interact head-to-tail (FIG. 2b), burying ˜600 Å² of surface area via hydrophobic and polar contacts (FIG. 2c-d). These interactions occur largely between side chains contributed by core helices α2 (between EF1/EF2) and α3 (between EF3/EF4) (FIG. 13). Residues at the dimer interface make direct contact with only one of the four metal-binding sites, EF3; three residues of EF3 in each monomer form a hydrogen bonding network with Arg100 of the other monomer (FIG. 2c), suggesting that occupancy and coordination geometry at this site may control oligomeric state.

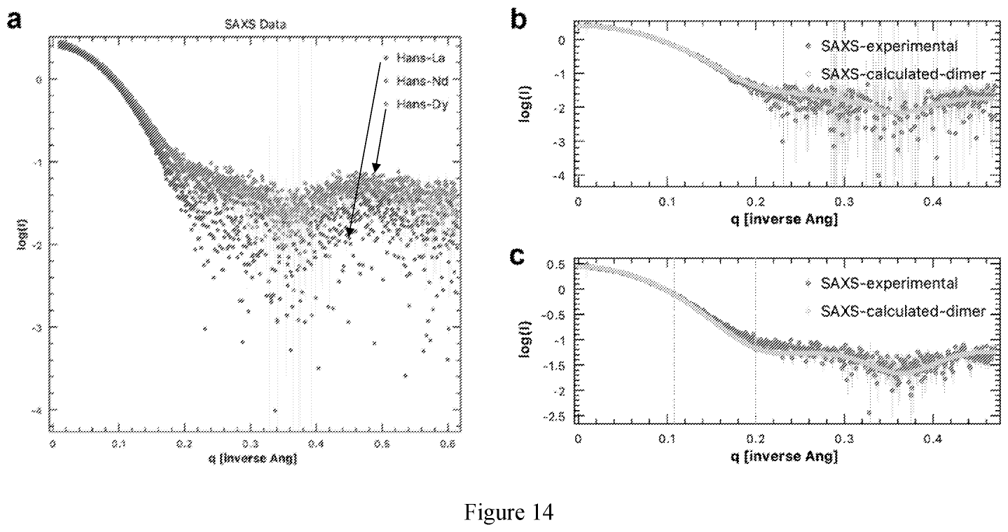

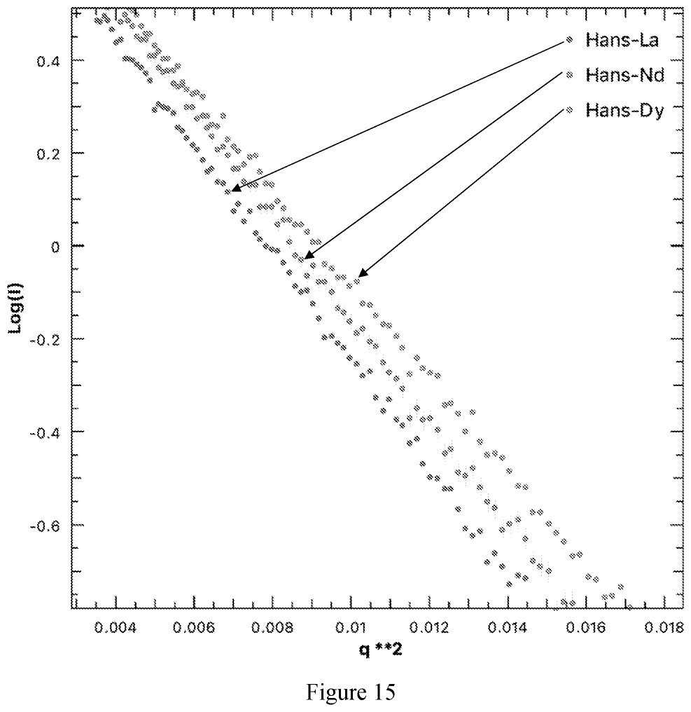

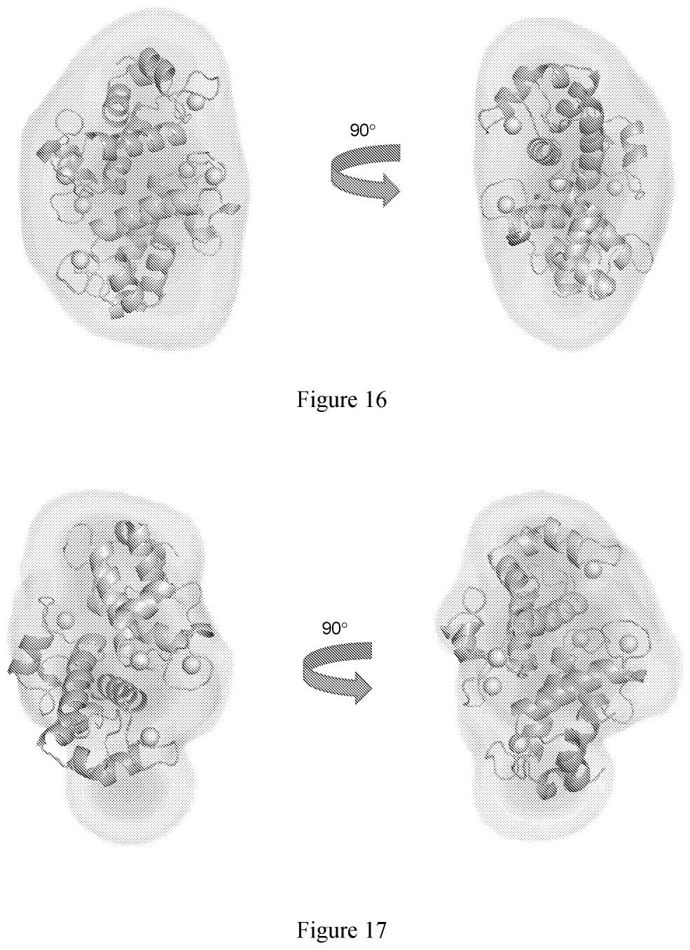

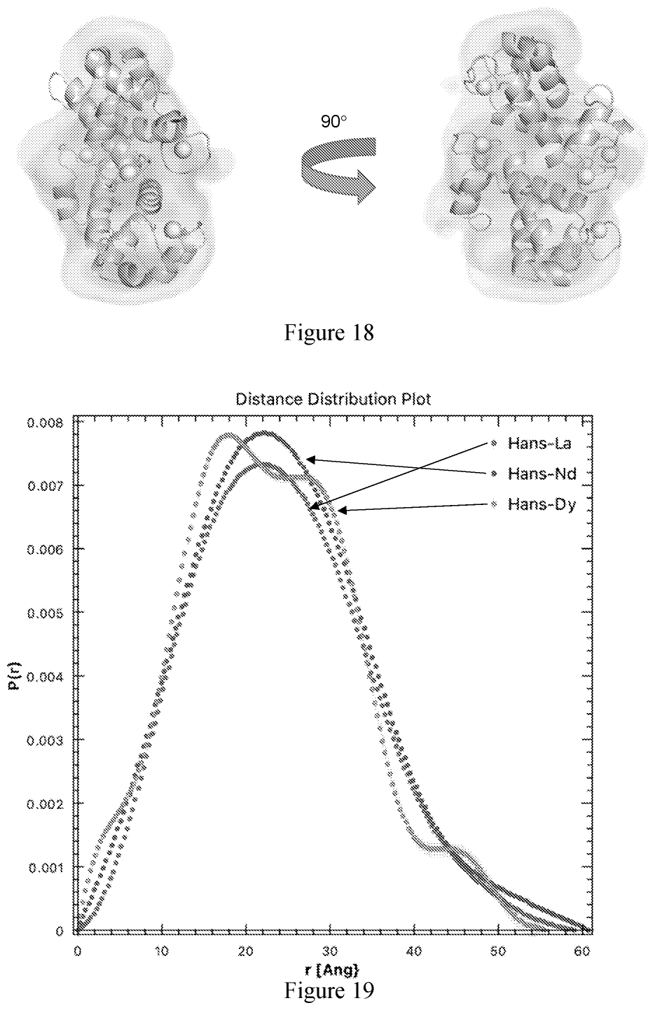

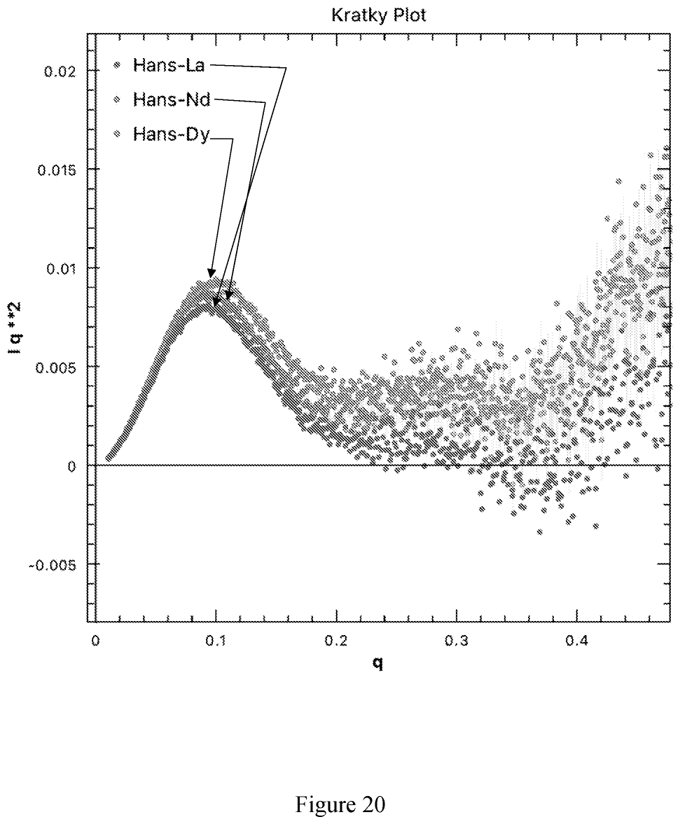

Hans-LanM and its complexes with 3 equiv. La^{III}, Nd^{III}, and Dy^{III} were also analyzed by small-angle x-ray scattering (SAXS) (FIGS. 14-15). The calculated solvent envelopes from the SAXS data fit well to the crystallographic Hans-LanM dimer for La^{III}-Hans-LanM, adequately for Nd^{III}-Hans-LanM, but poorly for Dy^{III}-Hans-LanM (FIG. 2e, FIGS. 16-18). The weaker dimerization of Dy^{III}-Hans-LanM is also supported by quantitative metrics, such as the Porod volume (Tables 5-6, FIGS. 19-20). Altogether, the biochemical and structural results indicate that Hans-LanM's dimerization equilibrium depends strongly on the particular RE bound.

Structural basis for dimerization. The structure of La^{III}-Hans-LanM additionally provides the first detailed view of the coordination environments in a lanmodulin, and indeed any natural biomolecule tasked with reversible lanthanide recognition. The prior NMR structure of Mex-LanM revealed the protein's unusual fold, but it could not provide molecular detail of the metal-binding sites. To understand the basis for LRE/HRE discrimination, a 1.4-Å-resolution structure of Dy^{III}-Hans-LanM was determined. Finally, a 1.01-Å-resolution structure of Nd^{III}-Mex-LanM is reported herein, which rationalizes Mex-LanM's shallower RE selectivity trend.

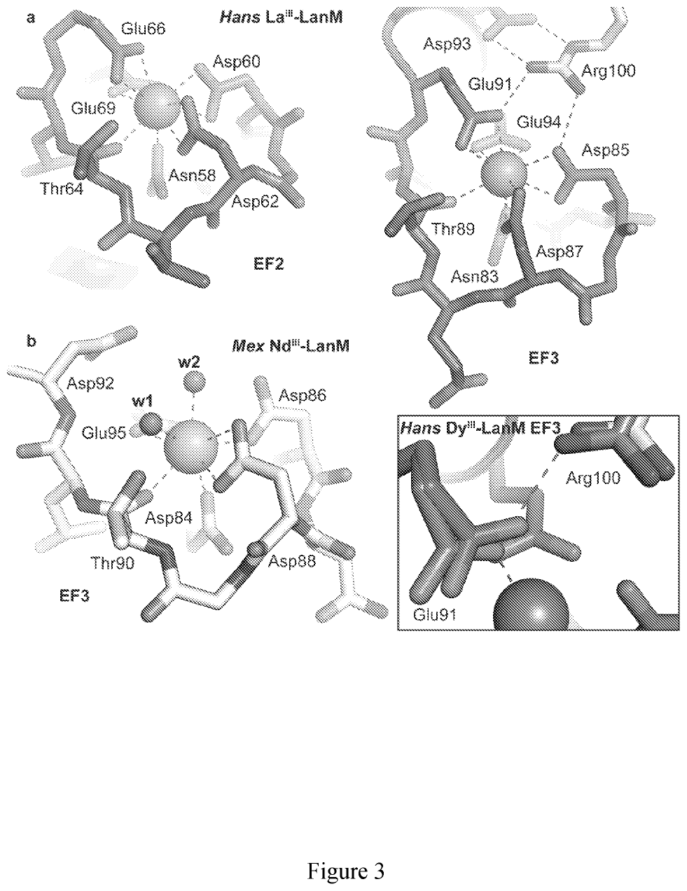

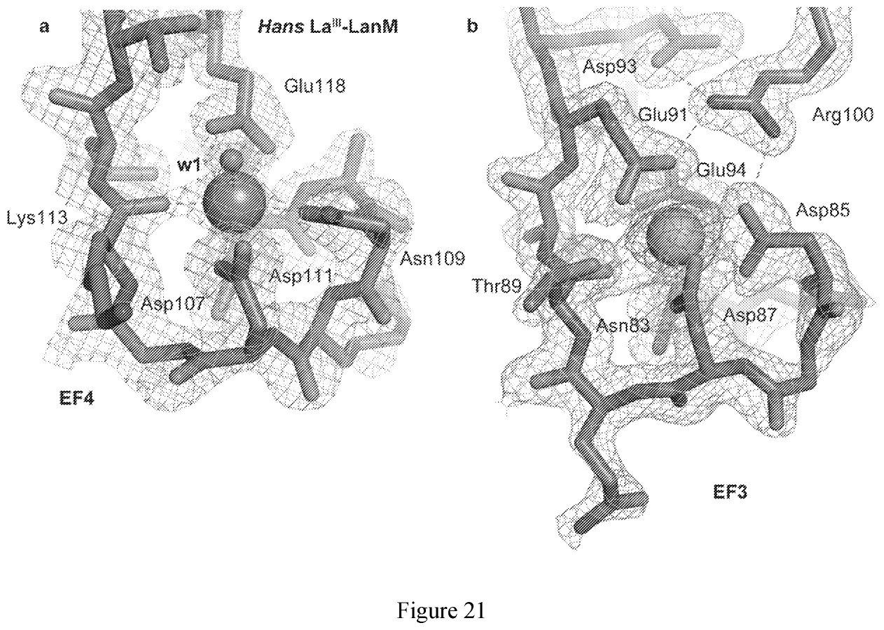

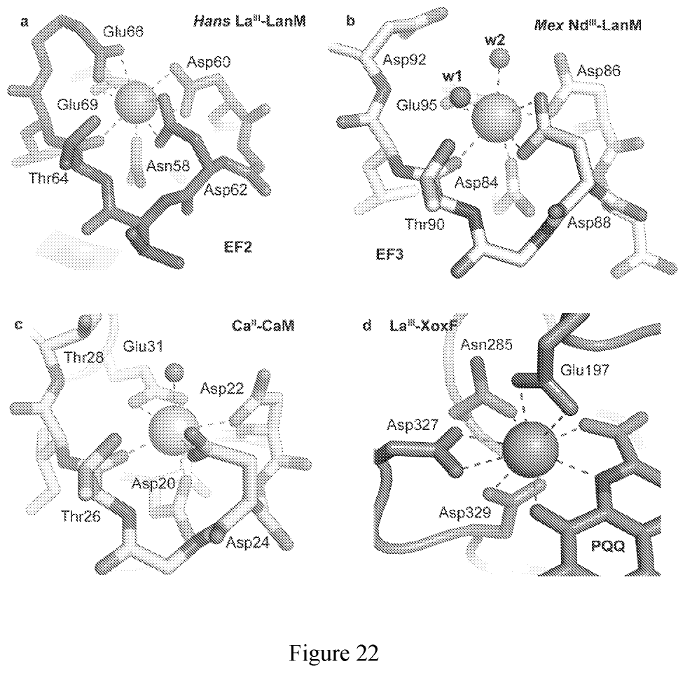

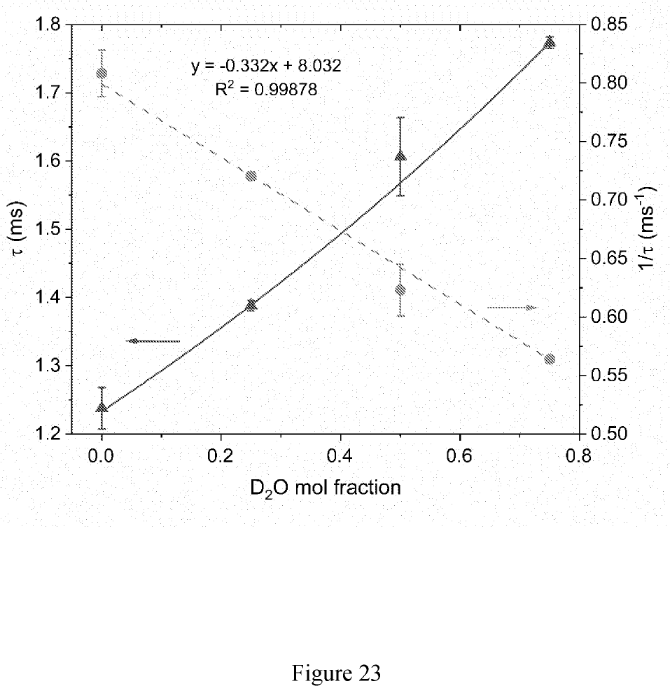

In La^{III}-Hans-LanM, EF1-3 are occupied by La^{III} ions (FIG. 52b-e). EF4 is structurally distinct, does not exhibit anomalous difference density consistent with La^{III}, and was modeled with Na^{I} (FIG. 21a). Each La^{III}-binding site is ten-coordinate, as observed in structures of lanthanide-dependent methanol dehydrogenases (FIG. 22). A monodentate Asn (Ni position), four bidentate Glu/Asp residues (D3, D5, E9, and E12), and a backbone carbonyl (T7/S7) complete the first coordination sphere in EF1-3 (FIG. 3a). Exogenous solvent ligands are not observed (FIG. 21b); luminescence studies of Eu^{III}-Hans-LanM to determine the number of coordinated solvent molecules (q) yielded q=0.11, consistent with the absence of solvent ligands in the X-ray structure (FIG. 23).

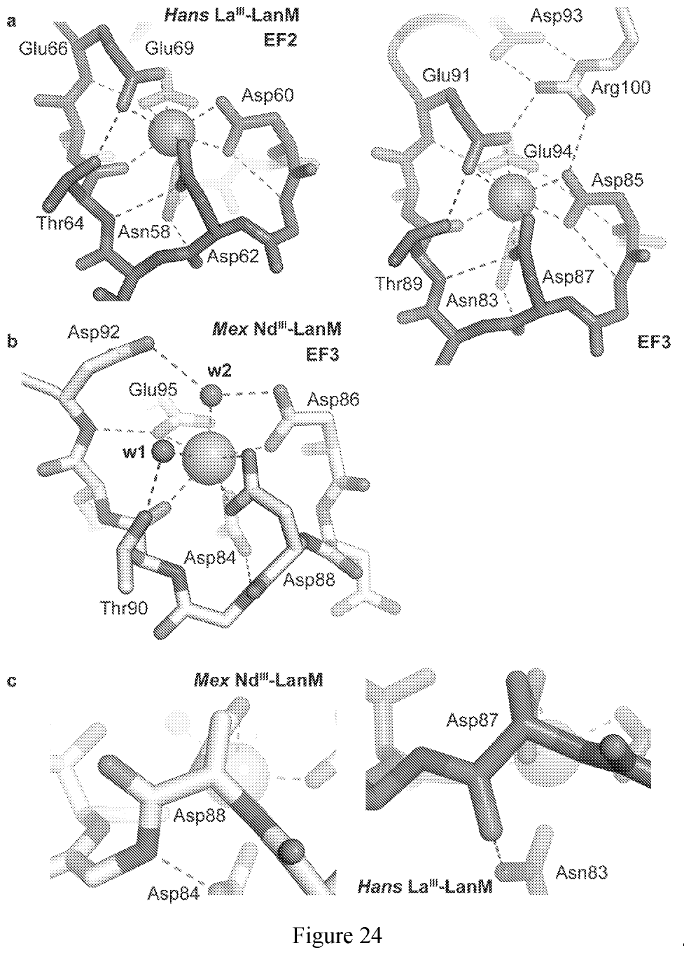

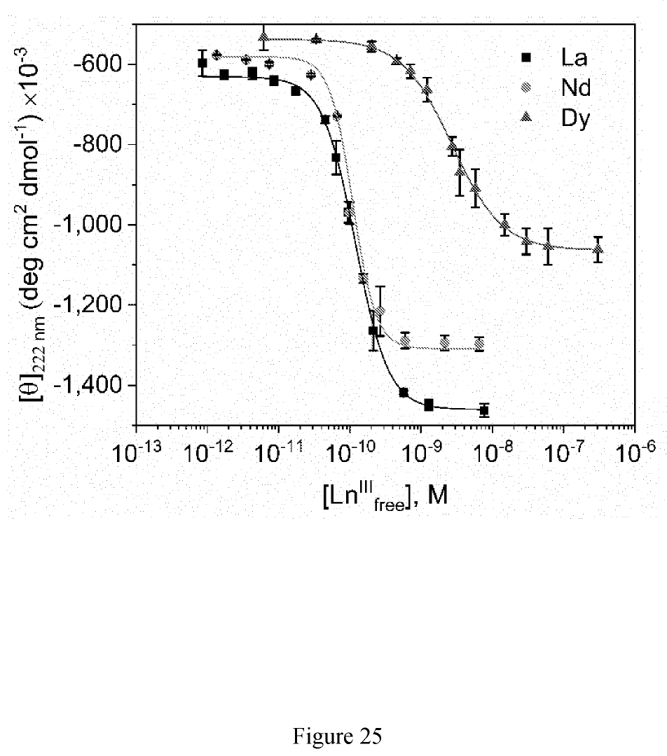

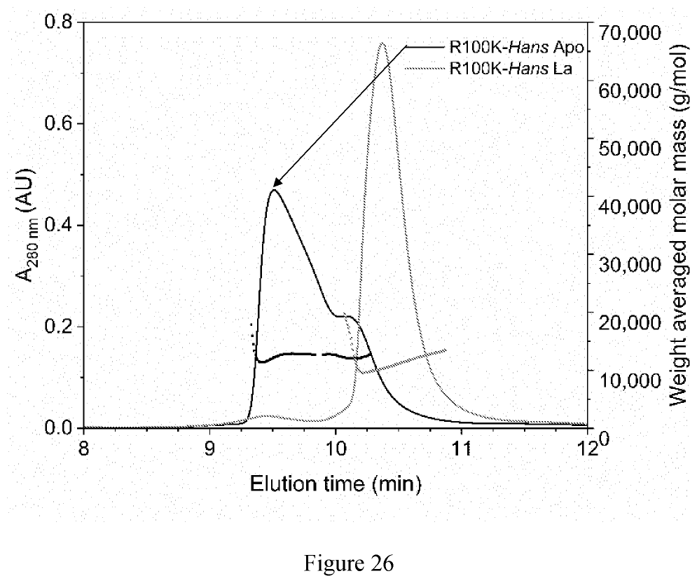

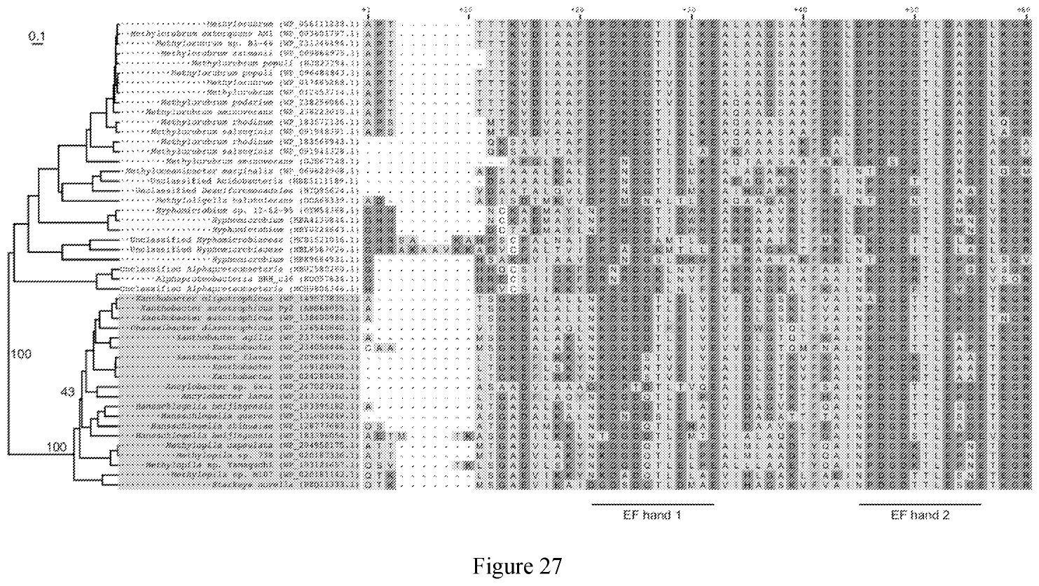

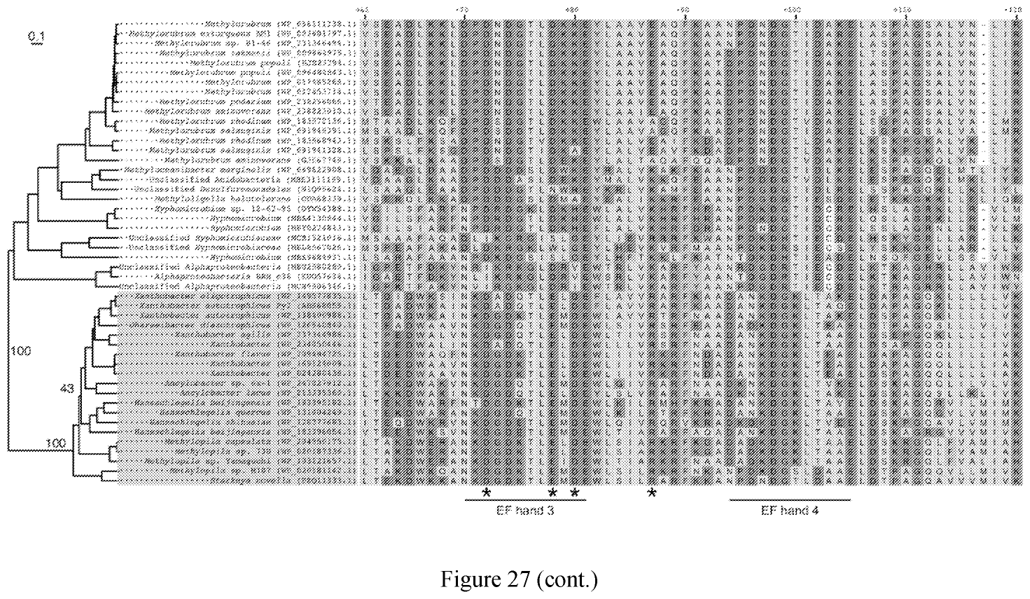

The lanthanide-binding sites in Hans-LanM additionally share extensive second-sphere interactions that may further constrain the positions of the ligands and the size of the metal-binding pore (FIG. 24). This phenomenon is most obvious in EF3, where the dimer interface mediates an extended hydrogen-bonding network involving several ligands. Arg100, contributed by the adjacent monomer, projects into the solvent-exposed side of EF3 to contact two carboxylate ligands, Asp85 (D₃) and Glu91 (E₉), enforcing their bidentate binding modes. Arg100 is also buttressed by Asp93 (EF3 D₁₁), unique to EF3 within Hans-LanM and not observed in Mex-LanM. The importance of this network in Hans-LanM dimerization was tested by making the minimal mutation, R100K. R100K-Hans-LanM had nearly identical K_{d,app} values and response to Nd^{III} and Dy^{III} as wild-type Hans-LanM, but the K_{d,app} for La^{II} was 2-fold weaker (Table 7, FIG. 25). SEC-MALS analysis indicated MWs of 10-13 kDa for apo-, La^{III}-, and Dy^{III}-R100K-Hans-LanM (FIG. 26, Table 8), indicative of increased monomerization, especially for the La^{III} complex, and suggesting that weaker dimerization may be responsible for the lower La^{III} affinity. All four residues comprising the Arg100-EF3 network are completely conserved in the Hans cluster (FIG. 27), suggesting that these interactions may contribute to dimerization in these lanmodulins.

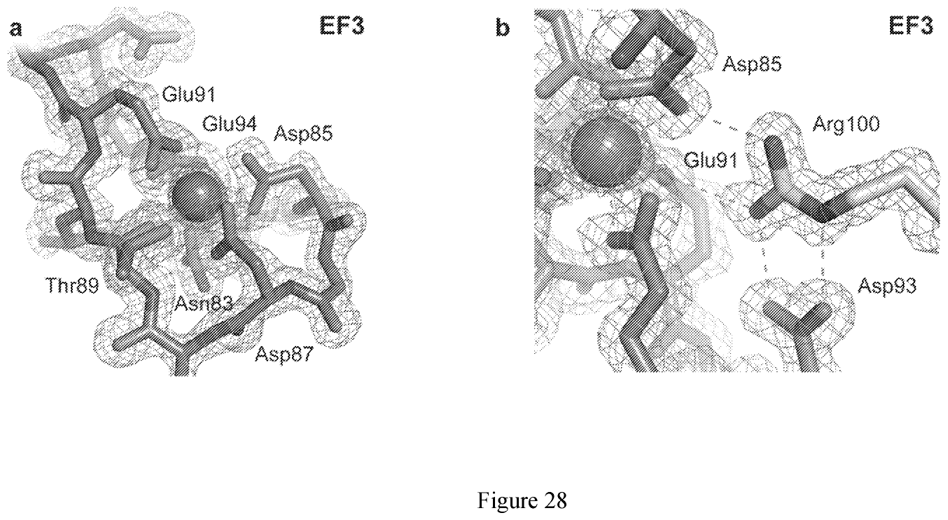

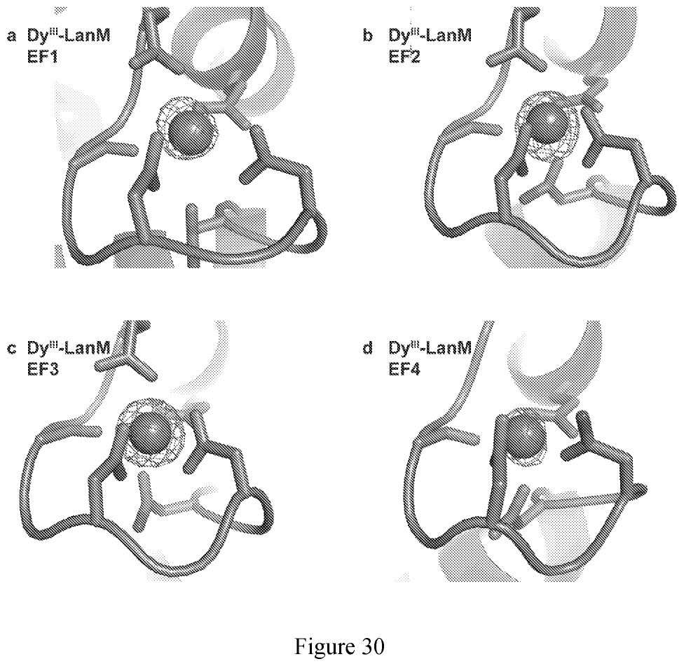

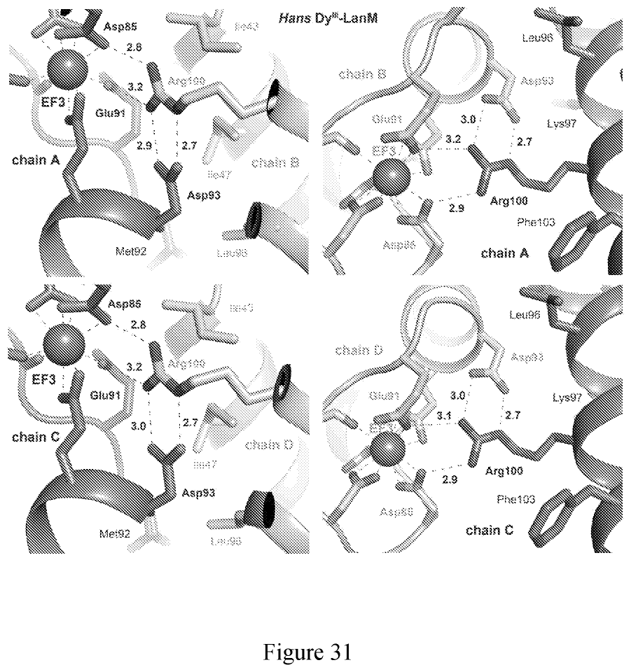

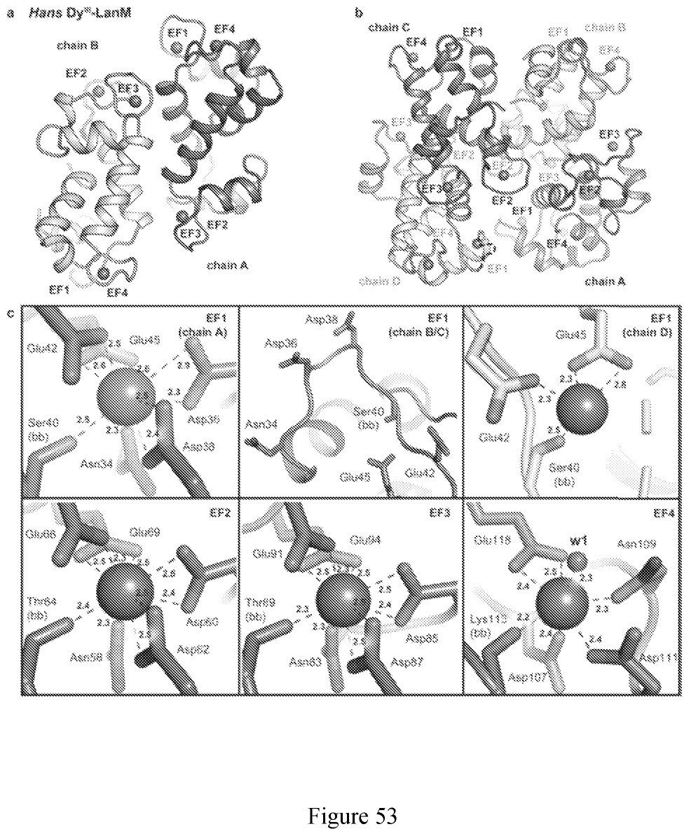

The structure of Dy^{III}-Hans-LanM confirms the significance of second-sphere control of ligand positioning (FIG. 53, FIG. 28-30, Table 9-10). The overall structure of Dy^{III}-Hans-LanM is largely superimposable with La^{III}-Hans-LanM, and the coordination spheres of the Dy^{III} ions in EF1-3 are similar to those in La^{III}-Hans-LanM (FIG. 3a inset), with the notable exception of E9 (e.g., Glu91 in EF3). This residue shifts from bidentate with La^{III} to monodentate with the smaller Dy^{III} ions, yielding a nine-coordinate distorted capped square antiprismatic geometry; the lower coordination number with a HRE ion is consistent with other RE complexes. In EF3, this carboxylate shift lengthens the distance between Arg100 and the proximal Oε of Glu91 from 2.9 Å (in La^{III}-Hans-LanM) to 3.2 Å(FIG. 31). The rearrangement of this second-sphere hydrogen bonding network suggests a structural basis for RE-dependent differences in K_{dimer} values.

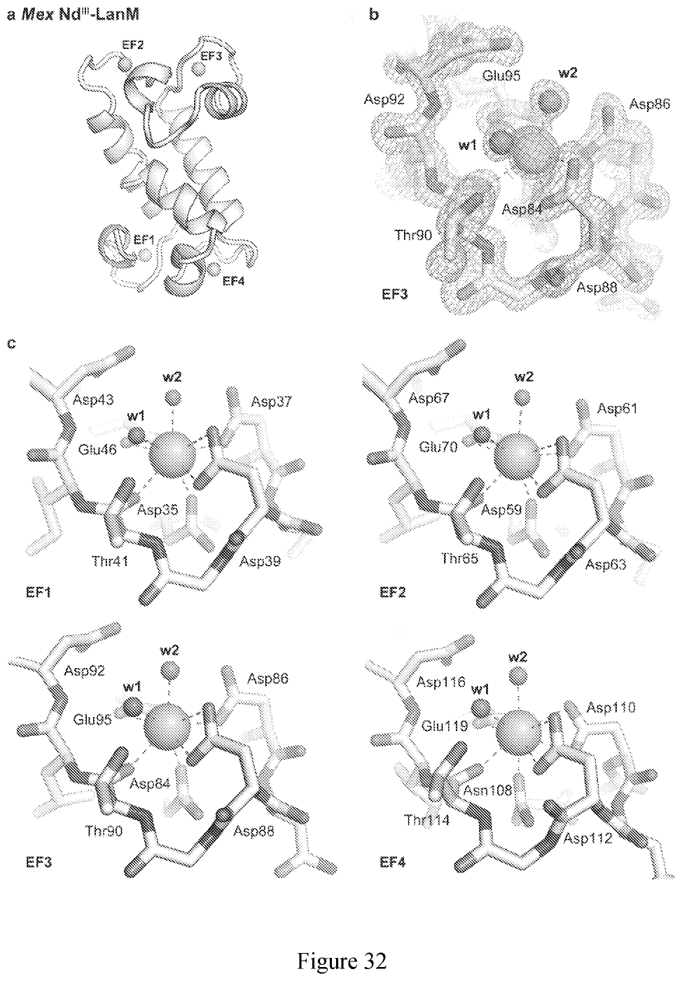

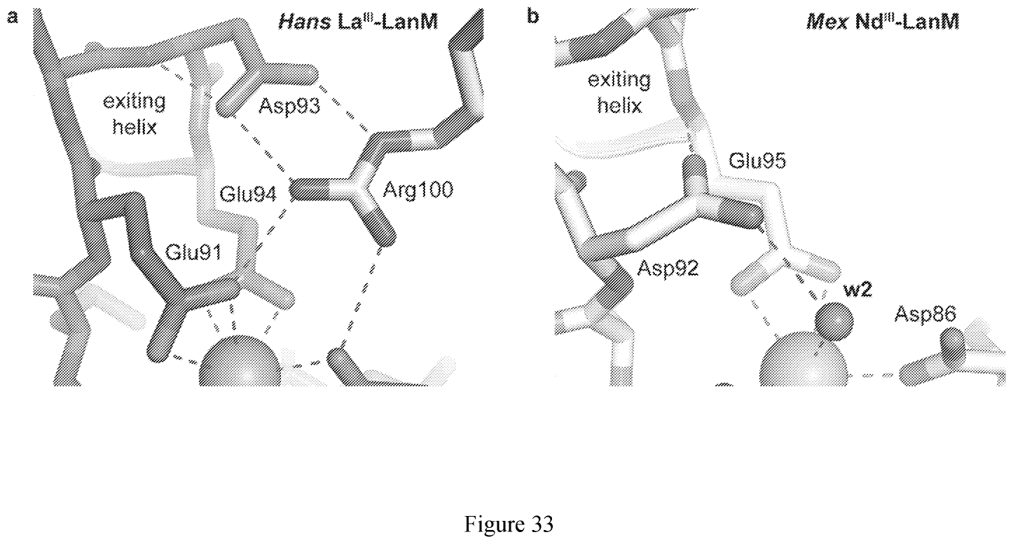

The metal-binding sites of Mex-LanM differ substantially from those of Hans-LanM. In Mex-LanM, all four EF-hands are occupied by nine-coordinate (EF1-3) or ten-coordinate (EF4) Nd^{III} ions, each including two solvent ligands, not present in Hans-LanM (FIG. 3b, FIG. 32). The observation of the two solvent molecules per metal site and the hydrogen bond to the D9 residue validates recent spectroscopic studies. The difference in coordination number between EF1-3 and EF4 is due to the D3 carboxylate being monodentate in EF1-3 but bidentate in EF4. Although the Mex-LanM Nd^{III} sites share the nine-/ten-coordination observed in Dy^{III}/La^{III}-Hans-LanM, they more closely resemble the seven-coordinate Ca^{II}-binding sites of calmodulin (FIG. 22). The increased coordination numbers in Mex-LanM relative to calmodulin result from bidentate coordination of D5 and an additional solvent ligand. These similarities suggest that much of LanM's unique 10⁸-fold selectivity for REs over Ca^{II} results from subtle differences in second-coordination-sphere and other more distal interactions. Finally, the exclusively protein-derived first coordination sphere in Hans-LanM, particularly due to coordination by E9, yields more extended hydrogen-bonding networks (FIGS. 24, 33) and likely enhances control over the radius of the binding site. Thus, the structures rationalize the extraordinary RE/non-RE selectivity of Mex-LanM and Hans-LanM while also accounting for their differences in LRE/HRE selectivity.

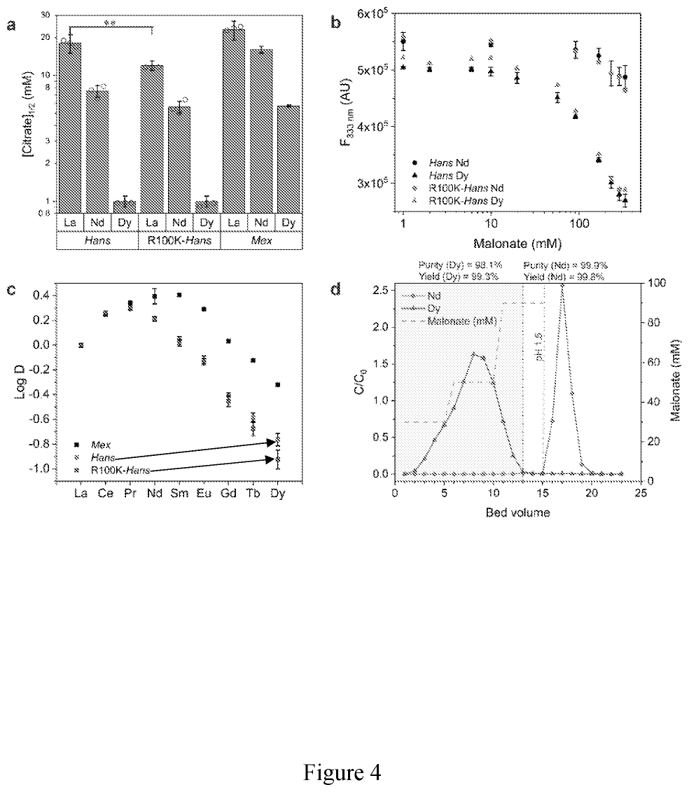

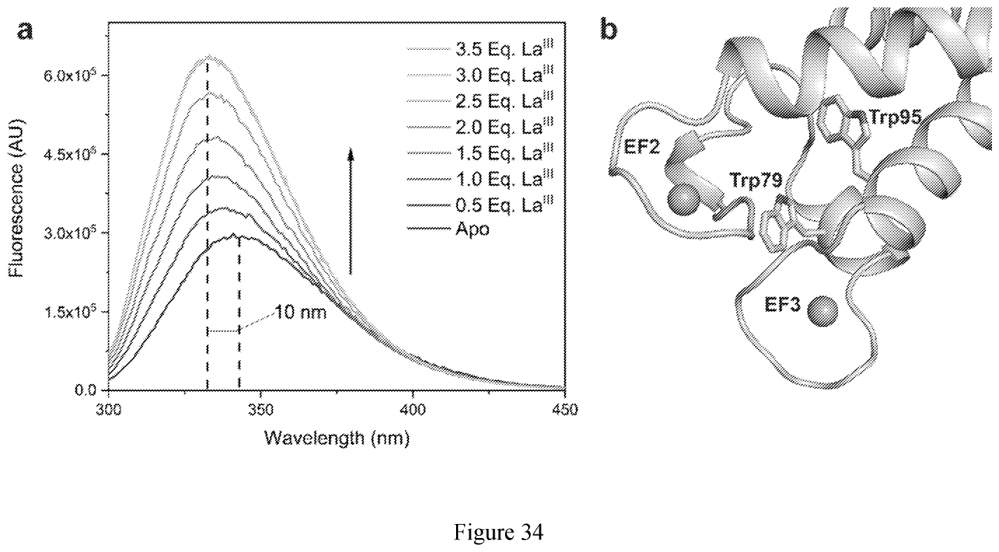

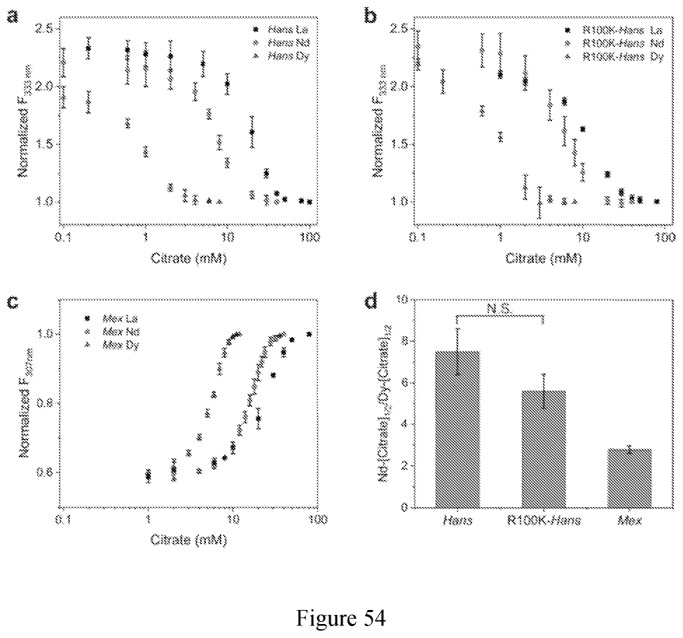

Single-stage Nd^{III}/Dy^{III} separation. The differences in stability and structure between Hans-LanM's LRE vs. HRE complexes suggested that Hans-LanM (wild-type and/or R100K) would outperform Mex-LanM in RE/RE separations. Separation of the RE pair of Nd^{III} and Dy^{III} used in permanent magnets was focused on. First, the stabilities of the wild-type- and R100K-Hans-LanM RE complexes were assayed against citrate, previously used as desorbent with Mex-LanM. RE-Hans-LanM complexes are generally less stable against citrate than those of Mex-LanM, as expected based on lower affinity (FIG. 1e), but the difference in stability between the Nd^{III}-Hans-LanM and Dy^{III}-Hans-LanM complexes—expressed as the ratio of citrate concentration required for 50% desorption of each metal ([citrate]_{1/2}), as reported by the fluorescence of Hans-LanM's two Trp residues (FIG. 34) —is also 2-fold greater than for Mex-LanM complexes (FIG. 4a, Table 11, FIG. 54). Furthermore, the R100K mutation significantly destabilizes Hans-LanM's La^{III} complex against citrate, whereas it only slightly affects the Nd^{III} complex and does not affect the Dy^{III} complex. This result confirms that dimerization selectively stabilizes Hans-LanM's LRE complexes (and especially the La^{III} complex), a factor abrogated by the R100K mutation. Using malonate, a weaker chelator than citrate, Dy^{III} can be readily desorbed from both Hans-LanM and R100K with 10-100 mM chelator without significant Nd^{III} desorption, suggesting conditions for Nd^{III}/Dy^{III} separation (FIG. 4b).

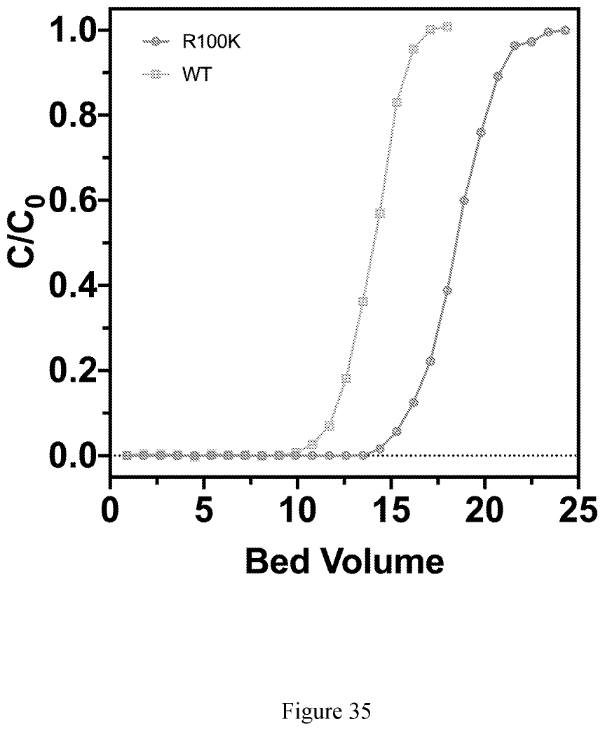

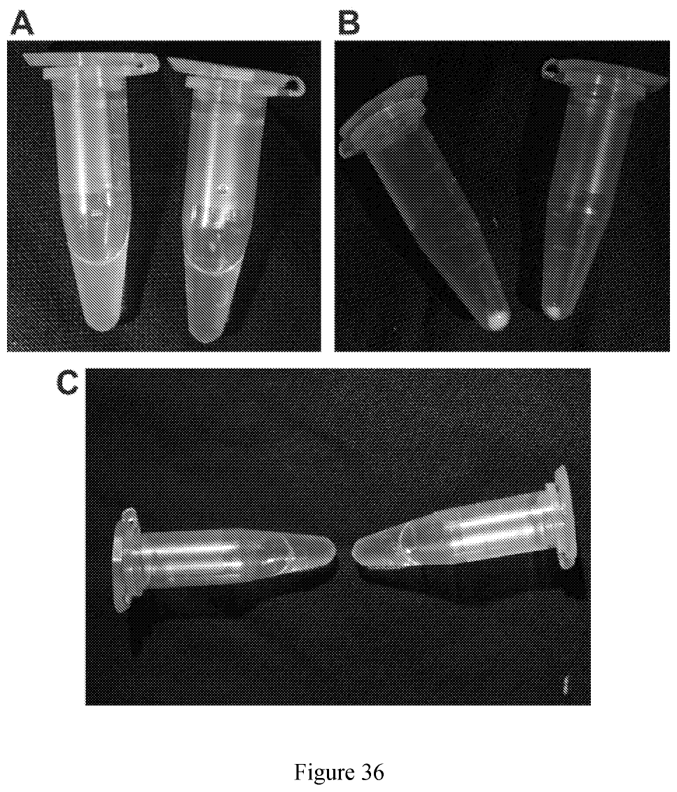

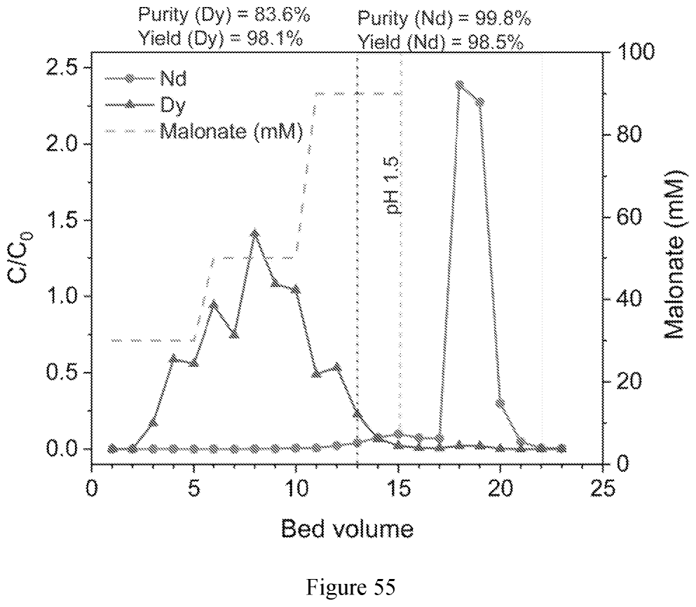

Although a 2-fold modulation of RE/RE selectivity by dimerization may seem small, such differences provide opportunity to decrease the number of separation stages, increasing efficiency of a separation process. Therefore, Hans-LanM and the R100K variant were immobilized via a C-terminal Cys residue on maleimide-functionalized agarose beads as described and tested for Nd^{III}/Dy^{III} separation. Immobilized Hans-LanM bound ˜1 equiv. RE, unlike in solution and compared with 2 equiv. for Mex-LanM and R100K-Hans-LanM (FIG. 35). Hans-LanM and R100K displayed similar separation ability in the La-Gd range—though R100K exhibits greater separation Gd-Dy—as determined by the on-column distribution ratios (D) of a mixed RE solution at equilibrium (Tables 12-14, Table 19, FIG. 4c). These Nd/Dy separation factors nearly double (Hans-LanM) and triple (R100K-Hans-LanM) that of Mex-LanM (Table 19). Immobilized Hans-LanM was loaded to 90% of breakthrough capacity with a model electronic waste mixture of 5% dysprosium and 95% neodymium and, guided by FIG. 4b, eluted with a short, stepwise malonate gradient, followed by complete desorption using pH 1.5 HCl. In a single purification stage, Dy was upgraded from 5% to 83% purity and Nd was recovered at 99.8% purity (both >98% yield) (FIG. 55). This significantly outperformed the comparable Mex-LanM-based process, which only achieved 50% purity in a first separation stage and required a second stage to obtain >98% purity. The immobilized R100K variant performed even better, achieving baseline separation of Dy^{III} and Nd^{III} to >98% purity and >99% yield in a single stage (FIG. 4d). The R100K variant's better performance was unexpected and may point to the unlikelihood of functional dimers on the column at this immobilization density (see FIG. 55 for a discussion). Thus, despite substantially improved performance versus Mex-LanM enabled by characterization of Hans-LanM's mechanism of dimerization, fully exploiting the dimerization phenomenon on-column may require tethering of two monomers on a single polypeptide chain, which is under investigation.

Conclusion. Biochemical and structural characterization of Hans-LanM's mechanism of metal-sensitive dimerization provides a new, allosteric mechanism for LRE/HRE selectivity in biology, extending concepts in dimer-dependent metal recognition recently emerging from synthetic lanthanide complexes and engineered transition metal-binding proteins and showing these principles are hard-wired into Nature. This disclosure shows that dimerization strength, and thus metal selectivity, can be rationally modulated. Hans-LanM evolved LRE-selective dimerization at physiological protein concentrations closer to those in biochemical assays (10-20 μM) disclosure herein rather than those on the column (˜3 mM). Therefore, leveraging dimerization in a separation process would be assisted by shifting dimerization sensitivity to the higher concentration regime, such as by tuning hydrophobic interactions at the dimerization interface. Furthermore, these studies establish that lanmodulins with as low as 33% identity have useful differences in metal selectivity. Finally, the solvent-excluded coordination spheres of Hans-LanM should outperform Mex-LanM in RE/actinide separation, luminescence-based sensing, and stabilization of hydrolysis-prone ions. Continued characterization of the coordination and supramolecular principles of biological f-element recognition will inspire design of ligands with higher RE/RE selectivities and their implementation in novel RE separation processes.

Bioinformatics methods. a) Protein and genome sequence data. The sequence of LanM from M. extorquens AMI was used as a query to conduct PSI-BLAST searches against the National Center for Biotechnology Information (NCBI) non-redundant protein sequence (nr) and metagenomic protein (env_nr) databases until convergence. The resulting 3,047 protein sequences were then manually curated for those that are less than 200 residues long, have at least one pair of EF-hands separated by less than 14 residues, and have four EF-hands. Signal peptides of LanM sequences were predicted using SignalP (v6.0), then removed before further analysis of the sequences. b) Construction of sequence similarity networks. The Enzyme Function Initiative-Enzyme Similarity Tool was used to calculate the similarities between all peptide sequence pairs with an E-value threshold of 1×10⁵. The resulting sequence similarity network (SSN) of 696 nodes and 241,853 edges was then constructed and explored using the organic layout via Cytoscape (v3.9.1) and visualized in R (v4.1.0). The edge percent identity threshold was gradually increased from 40% to 90% to yield distinct clusters. c) Multiple sequence alignment and phylogenetic analysis. LanM sequences were aligned using MUSCLE (v5.1) with default parameters. The model used for phylogeny construction was selected using ModelFinder in IQ-TREE (v2.2.0.3) with --mset set to beast2. Bayesian phylogeny was generated based on these results using BEAST (v2.6.7). The resulting phylogeny was evaluated using 10⁷ generations and discarding a burn-in of 25%, then visualized using ggtree (v3.2.1).

Expression and purification of Hans-LanM and its R100K variant. The gene encoding Hans-LanM, codon optimized for expression in E. coli without its native 23-residue signal peptide (see Table 15), was obtained from Twist Bioscience inserted into pET-29b(+) using the restriction sites NdeI/XhoI. Hans-LanM was overexpressed on 2-L scale and purified using the established protocol for Mex-LanM, with one modification: after the final size-exclusion chromatography (SEC) step, the protein was concentrated to 5 mL and dialyzed against 5 g Chelex-100 in 500 mL of 30 mM HEPES, 100 mM KCl, 5% glycerol, pH 8.4, to remove Ca^{II} and trace metal contaminants. This procedure resulted in approximately 15 mL of 550 μM protein, which was not concentrated further. The final yield was 45 mg protein per L culture. Protein concentrations were calculated using an extinction coefficient of 11000 M⁻¹ cm⁻¹, based on the ExPASy ProtParam tool. R100K-Hans-LanM was purified using the same procedure, yielding 30 mg protein per L culture.

Circular dichroism (CD) spectroscopy. CD spectra of Hans-LanM were collected as described, at 15 μM (monomer concentration) in Chelex-treated Buffer A (20 mM acetate, 100 mM KCl, pH 5.0), unless otherwise indicated. Buffered metal solutions were prepared as described. Additional details are described herein.

Preparation of protein samples for SEC-MALS and SAXS. Samples of wild-type Hans-LanM were prepared by adding 3.0 equivalents of metal slowly (0.5 equivalent at a time followed by mixing) to 1.0 mL of concentrated stock of Hans-LanM (550 μM). At these protein concentrations, slight precipitation was observed for LRE samples (e.g., La^{III}) whereas significant precipitation was observed for HRE samples (e.g., Dy^{III}). Samples were centrifuged at 12000×g for 2 min to remove precipitate and then purified using gel filtration chromatography (HiLoad 10/300 Superdex 75 μg, 1 mL loop, 0.8 mL/min) in Buffer B (30 mM MOPS, 100 mM KCl, 5% glycerol, pH 7.0). Hans-LanM-containing peaks (ranging from 12.0-15.0 mL elution volume) were collected to avoid the high-MW aggregate peaks, yielding 2.0 mL of metalated Hans-LanM ranging between 11 μM and 12 μM (1.37-1.53 mg/mL).

Samples of R100K-Hans-LanM do not form high molecular weight species or precipitate upon metal addition. To prepare samples of this protein, a 500 μM protein solution was diluted to 250 μM (3 mg/mL) in Buffer B containing 0.75 mM of a specific RECl₃, yielding a final solution of 3 mg/mL protein, with a 1:3 metal ratio, which was analyzed directly by SEC-MALS.

For calcium conditions, proteins were diluted to 250 μM (3 mg/mL), 5 mM CaCl₂) was added, the samples were incubated at room temperature for 1 h. The buffer used for SEC-MALS was the same as above, except that it also contained 5 mM CaCl₂).

In-line size-exclusion chromatography and multi-angle light scattering (SEC-MALS). SEC-MALS experiments were conducted using an Agilent 1260 Infinity II HPLC system equipped with an autosampler and fraction collector, and the Wyatt SEC hydrophilic column had 5 μm silica beads, a pore size of 100 Å, and dimensions of 7.8×300 mm. Wyatt Technology DAWN MALS and Wyatt Optilab Refractive Index (RI) detectors were used for analyzing the molar mass of peaks that eluted from the column. The SEC-MALS system was equilibrated for 5 h with Buffer B. The system was calibrated with bovine serum albumin (BSA, monomer MW: 66 kDa) in the same buffer and normalization and alignment of the MALS and RI detectors were carried out. A volume of 15 μL of each sample was injected at a flow rate of 0.8 mL/min with a chromatogram run time of 25 min. Data were analyzed using the ASTRA software (Wyatt). When SAXS analysis was desired, a second run was performed with 150 μL protein (˜4 mg/mL) injected, and 200 μL fractions of the main peak were collected. BioSAXS data were subsequently collected in triplicate.

Isothermal titration calorimetry. The dissociation constants for the dimers of apo, La^{III}-bound, and Dy^{III}-bound Hans-LanM were determined by dilutive additions of a concentrated protein stock, followed using ITC on a TA Instruments Low-volume Auto Affinity ITC. The syringe contained 300 μM protein (apo or 2 equiv. Dy^{III} bound) or 150 μM or 540 μM (2 equiv. La^{III} bound), and the cell contained 185 μL of a matched buffer (30 mM MOPS, 100 mM KCl, pH 7.0). Titrations were carried out at 30° C. Titrations consisted of a first 0.2 μL injection followed by 17×2 μL injections, unless otherwise noted, with stirring at 125 rpm and 180 s equilibration time between injections. The heats were fitted using NanoAnalyze using the “Dimer Dissociation” model, yielding the dimer dissociation constant (K_{dimer}), enthalpy of dissociation (ΔH), and entropy of dissociation (ΔS). All parameters are shown in Table 18.

K_{dimer} is defined as the dissociation constant for the equilibrium D⇄2M, such that K_{dimer}=[M]²/[D], where [D] is the concentration of the dimer and [M] is the concentration of the monomer, and the total protein concentration [P](as measured using the extinction coefficient for the monomer) is given by [P]=[M]+2[D]. Therefore, K_{dimer}=2[M]²/([P]−[M]) or

$${2 [ M ]}^{2} + {K}_{dimer} [ M ] - {K}_{d imer} [ P ] = 0 \qquad ( Eq . 1 )$$

This equation can be used to estimate monomer and dimer concentrations during SEC-MALS experiments, using K_{dimer} values calculated from ITC experiments and [P] from the SEC-MALS trace. This equation can also be used to estimate the maximum possible K_{dimer} for La^{III}-bound protein, given the SEC-MALS data.

BioSAXS. Small-angle X-ray scattering (BioSAXS) was collected on RE-complexed Hans-LanM, at protein concentrations given in Table 5 using equipment and under conditions described herein.

The forward scattering I(0) and the radius of gyration (R_{g}) are listed in Table 5 and were calculated using the Guinier approximation, which assumes that at very small angles (q<1.3/R_{g}) the intensity is approximated as I(q)=I(0)exp[−1/3(qR_{g})²]. In the La^{III}-, Nd^{III}-, and Dy^{III}-bound conditions, this agrees with the calculated size of 17.9 Å for the crystallographic dimer. The molecular mass was estimated using a comparison with SAXS data of a BSA standard. The data files were analyzed for Guinier R_{g}, maximum particle dimension (Dₘₐₓ), Guinier fits, Kratky plots, and pair distance distribution function using the ATSAS software. GNOM, within ATSAS, was used to calculate the pair-distance distribution function P(r), from which R_{g} and Dₘₐₓ were determined. Solvent envelopes were computed using DENSS. The theoretical scattering profiles of the constructed models were calculated and fitted to experimental scattering data using CRYSOL. OLIGOMER was used to estimate the monomer and dimer fractions.

Preparation of protein samples for crystallography. To Hans-LanM (2 mL, 1.16 mM, Buffer B), 3.0 equiv. LaCl₃ or DyCl₃ were added slowly, 0.5 equivalents at a time with mixing, to minimize precipitation. Precipitate was removed by centrifugation at 12000×g for 2 min. Any soluble aggregates were removed and the protein was exchanged into buffer lacking glycerol (Buffer C: 30 mM MOPS, 50 mM KCl, pH 7.0) by gel filtration chromatography (HiLoad 16/600 Superdex 75 μg, 1 mL loop, 0.75 mL/min). The peak in the 70-85 mL range was pooled, and the fractions were concentrated to ˜500 μL with a final concentration of ˜1.3 mM.

Mex-LanM was purified as described and was exchanged into Buffer C prior to crystallization. The protein was loaded with 3.5 equivalents of Nd^{III} (NdCl₃).

General crystallographic methods. Diffraction datasets were collected at the Life Sciences Collaborative Access Team (LS-CAT) ID-G beamline and processed with the HKL2000 package. In all structures, phase information was obtained with phenix.autosol via the single-wavelength anomalous diffraction (SAD) method, in which lanthanide ions identified with HySS were used as the anomalous scatterers. Initial models were generated with phenix.autobuild with subsequent rounds of manual modification and refinement in Coot and phenix.refine. In the final stages of model refinement, anisotropic displacement parameters (ADP) and occupancies were refined for all lanthanide sites. Model validation was performed with the Molprobity server. Figures were prepared using the PyMOL molecular graphics software package (Schrödinger, LLC).

La-bound Hans-LanM structure determination. Crystals were obtained by using the sitting drop vapor diffusion method, in which 1 μL of protein solution (15 mg/mL) was mixed with 1 μL 10 mM tri-sodium citrate, pH 7.0, and 27% (w/v) PEG 6000 in a 24-well plate from Hampton Research (cat. no. HR1-002) at room temperature. Thin plate-shaped crystals appeared in three days. Crystals suitable for data collection were mounted on rayon loops, soaked briefly in a cryoprotectant solution consisting of the well solution supplemented with 10% ethylene glycol, and flash-frozen in liquid N2.

La^{III}-loaded Hans-LanM crystallized in the P2₁ space group (β=90.024°) with 4 monomers in the asymmetric unit. The initial figure of merit (FOM) and Bayesian CC were 0.563 and 0.56, respectively. The final model consists of residues 24-133 in each chain, 12 La^{III} ions (3 per chain in the first, second, and third EF-hands), 4 Na^{I} ions (1 per chain in the fourth EF-hand), 273 water molecules, and 2 molecules of citrate. Of the residues modeled, 100% are in allowed or preferred regions as indicated by Ramachandran statistical analysis.

Dy-bound Hans-LanM structure determination. Crystals were obtained by using the sitting drop vapor diffusion method, in which 1 μL of protein solution (15 mg/mL) was mixed with 1 μL 25 μM tri-sodium citrate, pH 7.0, and 27% (w/v) PEG 6000 in a 24-well plate from Hampton Research (cat. no. HR1-002) at room temperature. Thin plate-shaped crystals appeared within 1 month. Crystals suitable for data collection were mounted on rayon loops, soaked briefly in a cryoprotectant solution consisting of the well solution supplemented with perfluoropolyether cryo oil from Hampton Research (cat. no. HR2-814), and flash-frozen in liquid N2.

Dy^{III}-loaded Hans-LanM crystallized in the P21 space group (β=93.567°) with 4 monomers in the asymmetric unit. The initial FOM and Bayesian CC were 0.748 and 0.58, respectively. The final model consists of residues 24-133 in each chain (except for chain D, for which residues 34-38 cannot be modeled), 14 Dy^{III} ions (4 in chains A and D, 3 in the second, third, and fourth EF-hands of chains B and C), and 656 water molecules. Of the residues modeled, 100% are in allowed or preferred regions as indicated by Ramachandran statistical analysis. Collection of anomalous datasets is described herein.

Nd-bound Mex-LanM structure determination. Crystals were obtained by using the sitting drop vapor diffusion method, in which 1 μL of protein solution (35 mg/mL) was mixed with 1 μL 0.1 M ammonium sulfate, 0.1 M Tris pH 7.5, and 20% (w/v) PEG 1500 in a 24-well plate from Hampton Research (cat. no. HR1-002) at room temperature. Thin plate-shaped crystals appeared within six months. Crystals suitable for data collection were mounted on rayon loops, soaked briefly in a cryoprotectant solution consisting of the well solution supplemented with perfluoropolyether cryo oil from Hampton Research (cat. no. HR2-814), and flash-frozen in liquid N₂.

Nd^{III}-loaded Mex-LanM crystallized in the P2₁2₁2₁ space group with one monomer in the ASU. The initial FOM and Bayesian CC were 0.799 and 0.56, respectively. The final model consists of residues 29-133, 4 Nd^{III} ions, and 171 water molecules. Of the residues modeled, 100% are in allowed or preferred regions as indicated by Ramachandran statistical analysis.

Fluorescence spectroscopy. All fluorescence data were collected using a Fluorolog-QM fluorometer in configuration 75-21-C(Horiba Scientific) equipped with a double monochromator on the excitation arm and single monochromator on the emission arm. A 75 W Xenon lamp was used as the light source for steady-state measurements and a pulsed Xenon lamp was used for time-resolved measurements. 10-mm quartz spectrofluorometry cuvettes (Starna Cells, 18F-Q-10-GL14-S) were used to collect data at 90° relative to the excitation path.

Fluorescence lifetime measurements were carried out using established methods. In short, a solution of Hans-LanM with 2 equiv. Eu^{III} added, totaling 4.5 mL, was prepared in 100% H₂O matrix (Buffer: 25 mM HEPES, 75 mM KCl, pH 7.0). Half of this initial protein mixture (2.25 mL) was retained for future use while the remainder was exchanged to D20 through lyophilization to remove H₂O and resuspension in 99.9% D20 two times. The resulting protein solutions (in 100% H₂O and ˜99% D20) were mixed in varying ratios to produce D20 contents of 0%, 25%, 50%, and 75%. The protein concentration was 20 μM. For each sample, the luminescence decay time constant (τ) was measured (λₑₓ=394 nm, λₑₘ=615 nm) with 5000 shots over a time span of 2500 μs. τ was determined using the FelixFL Powerfit-10 software (Horiba Scientific) using a single exponential fit. 1/τ was plotted against percent composition of D20 and the slope of the resulting line (m) was determined. The q value was determined using the following equation (Eq. 2):

$$q = 1.11 [ {τ}_{H 2 O}^{- 1} - {τ}_{D 2 O}^{- 1} - 0.31 + 0.45 {n}_{OH} + 0.99 {n}_{NH} + 0.075 {n}_{O - CNH} ] \qquad ( Eq . 2 )$$

where τ⁻¹_{H2O} and τ⁻¹_{D2O} are the inverses of the time constants in 100% H₂O and D₂O, respectively (the latter extrapolated using the equation of the fitted line), in ms⁻¹; and n_{OH}=0, n_{NH}=0, and n_{O-CNH}=1 (resulting from the metal-coordinated Asn residues), based on the Hans-LanM crystal structures. This equation simplifies to Eq. 3:

$$q = 1.11 [ - m - 0.31 + 0.075 ] \qquad ( Eq . 3 )$$

For fluorescence competition experiments, a solution of 20 μM Hans-LanM or the R100K variant was prepared in Buffer A (pH 5.0) with 2 equiv. of metal (4 μM). Fluorescence emission spectra were collected with settings: λₑₓ=278 nm, λₑₘ 300-420 nm, integration time=0.5 sec, step size=1 nm. Titrations were carried out through addition of at least 0.6 μL of titrant (from concentrated stock solutions of 10 mM-1 M citrate or malonate, pH 5.0). Spectra were corrected for dilution. Each experiment was performed in triplicate.

Purification of Cys-containing variants. R100K-Hans-LanM-Cys was expressed and purified as described for Mex-LanM-Cys, with a final yield of 50 mg protein/L culture. For Hans-LanM-Cys, the protein was purified by incorporating the same modifications from above, minus the dialysis step, to previously described Mex-LanM-Cys purification, except that the SEC step was run using a reducing buffer (30 mM MOPS, 100 mM KCl, 5 mM TCEP, pH 7.0) with 5 mM EDTA, and frozen under liquid N2 prior to immobilization.

Maleimide functionalization of agarose beads. The maleimide functionalization of amine-functionalized agarose beads was described previously.

Immobilization of Hans-LanM and the R100K variant. R100K-Hans-LanM immobilization was performed using a thiol-maleimide conjugation reaction as previously described. In the case of Hans-LanM, a final protein concentration of ˜0.4 mM (8 mL) was combined with 1 mL of maleimide-microbeads and the conjugation reaction was carried out for 16 h at room temperature. Unconjugated Hans-LanM was removed by washing with coupling buffer and the Hans-LanM microbeads were stored in coupling buffer for subsequent tests. To quantify Hans-LanM immobilization yield, Pierce™ BCA Protein Assay (ThermoFisher Scientific) was used to determine the LanM concentration in the reaction solution before and after the conjugation reaction as previously described.

Batch experiment to determine separation factors. LanM-immobilized microbeads were washed with DI-water. Feed solution (5 mL, equimolar REs La-Dy, 3 mM total, pH 5.0) was added to 1 mL microbeads and incubated for 2 h. The liquid at equilibrium was collected and RE concentrations were determined by ICP-MS as [M]_{ad}. Then 4 mL 0.1 M HCl was used to desorb REs from the microbeads, and concentrations were measured by ICP-MS as [M]_{de}.

The RE distribution factor (D) between the LanM phase and the solution phase was calculated as:

$$D = {[ M ]}_{L a n M} / {[ M ]}_{L i q u i d} \qquad ( Eq . 4 )$$

where [M]_{LanM} and [M]_{Liquid} are the molar concentrations of each metal ion in the LanM phase and the solution phase at equilibrium, respectively. To account for the free liquid that absorbed on the agarose microbeads, the following correction was applied: [M]_{Liquid}=[M]_{ad}; [M]_{LanM}=(4×[M]_{de}−[M]_{ad})/4.

The separation factor (SF) is defined as:

$$SF = {D}_{RE 1} / {D}_{RE 2} \qquad ( Eq . 5 )$$

where D_{RE1} and D_{RE2} are distribution factors of RE1 and RE2, respectively.

Breakthrough column experiments. Columns were filled and run, and metal concentrations analyzed, as described in our previous work; details are contained herein.

For the RE pair separation experiments, the metal ion purity and yield are defined as:

$$Purity ( RE 1 ) = {C}_{RE 1} / ( {C}_{RE 1} + {C}_{RE 2} ) \qquad ( Eq . 6 )$$

$$Yield ( RE 1 ) = ( RE 1 recovered ) / ( Total RE 1 loaded ) \qquad ( Eq . 7 )$$

where C_{RE1} and C_{RE2} are the molar concentrations of RE1 and RE2, respectively.

Table 17. Characterization of Hans- and Mex-LanM metal complexes using SEC-MALS. The concentrations of the protein samples loaded to the column were: 1.2-1.5 mg/mL for apo- and RE-bound Hans-LanM, 3 mg/mL for Ca^{II}-bound Hans-LanM, and 3 mg/mL for Mex-LanM. For RE-containing samples, protein was pre-incubated with 3 equiv. of the appropriate RE^{III} ion. In the case of Ca^{II}, 5 mM CaCl₂) was added to the running buffer. The apoprotein elutes in two peaks, the first being a minor contribution (10% of protein, 56.5 kDa) and the second being the major peak (90% of protein, 12.9 kDa). See Materials and Methods for full details of sample preparation. The values for Hans-LanM are plotted in FIG. 2a; raw data for La, Nd, and Dy are shown in FIG. 9.

| | | | | | |
|---|---|---|---|---|---|
| | | Molecular | | | Average protein |
| | Peak retention | weight | | Hydrodynamic | concentration in |
| Metal ion | time (min) | (kDa) | Polydispersity | radius (nm) | elution peak (μM) |

Hans-LanM

| | | | | | |
|---|---|---|---|---|---|
| Apo | 7.9 | 56.5 | 1.034 | — | |
| | 10.05 | 12.9 | 1.001 | 1.11 | 14.1 |
| Ca^{II} | 9.7 | 14.7 | 1.000 | 1.48 | |
| La^{III} | 10.05 | 24.5 | 1.084 | 1.55 | 18.4 |
| Nd^{III} | 10.1 | 22.1 | 1.048 | 1.39 | |
| Gd^{III} | 9.9 | 24.6 | 1.003 | 1.28 | |
| Tb^{III} | 9.75 | 21.6 | 1.037 | 1.25 | |
| Dy^{III} | 10.3 | 15.5 | 1.038 | 1.23 | 18.9 |
| Ho^{III} | 9.9 | 15.2 | 1.006 | 1.43 | |

Mex-LanM

| | | | | |
|---|---|---|---|---|
| Apo | 8.8 | 11.1 | 1.031 | 1.52 |
| Ca^{II} | 10.1 | 11.4 | 1.008 | 1.21 |
| La^{III} | 10.5 | 11.5 | 1.002 | 1.22 |
| Nd^{III} | 10.5 | 11.8 | 1.003 | 1.21 |
| Dy^{III} | 10.6 | 11.4 | 1.008 | 1.28 |

Table 18. Thermodynamic parameters for apo- and Dy^{III}-bound Hans-LanM, obtained by fitting ITC thermograms to the dimer dissociation model. These values cannot be determined for the La^{III}-bound protein because no changes in the measured heats are observed during the titration experiment (FIG. 12). Values are reported as the mean with standard deviation from three independent titrations.

| | K_{dimer} (μM) | ΔH (kcal/mol) | ΔG (kcal/mol) | ΔS (cal/mol/K) |
|---|---|---|---|---|
| Apo | 117(21) | 25.4(6) | 5.5(1) | 66(2) |
| Dy^{III} | 60(30) | −5.3(6) | 5.9(3) | −37(4) |

Table 19. Distribution factors (D) and separation factors (SF) for equilibration of a binary Nd/Dy solution using a Hans-LanM column or R100K-Hans-LanM column. The volumes of the Hans-LanM column and R100K-Hans-LanM column were 0.9 mL and 0.7 mL, respectively. The feed solution for this experiment was 5.0 mL with a composition of 1.42(4) mM Nd and 1.62(32) mM Dy, as determined by ICP-MS analysis. The pH was 5.0. This experiment confirms that the immobilized R100K variant exhibits better on-column separation properties than wild-type Hans-LanM. See Table S12 legend for details on uncertainty values in parentheses.

| | |
|---|---|
| D | SF |

Hans-LanM

| | | |
|---|---|---|
| Nd^{III} | 1.64(8) | 8.12(40) |
| Dy^{III} | 0.20(1) | |

R100K-Hans-LanM

| | | |
|---|---|---|
| Nd^{III} | 2.63(20) | 12.7(1.3) |
| Dy^{III} | 0.21(2) | |

General considerations. Chemical reagents were obtained, at the highest purity available, from Millipore Sigma, unless noted otherwise. Chemically competent E. coli BL21 (DE3) cells were obtained from NEB. Biochemical experiments and column-based experiments were performed using RE chloride salts and buffers obtained at a minimum purity of 99.9% from Millipore Sigma. Anion exchange chromatography was performed using Q Sepharose Fast Flow resin obtained from Millipore Sigma. Automated protein chromatography was carried out on a GE Healthcare Biosciences Akta Pure fast protein liquid chromatography (FPLC) system using either a HiLoad Superdex 75 pg 16/600 column for preparative scale or a Superdex 75 pg Increase 10/300 GL column for analytical scale. REs were quantified using inductively coupled plasma mass spectrometry (ICP-MS; Thermo Scientific iCAP RQ) with He in KED mode. The ICP-MS is housed in the Laboratory for Isotopes and Metals in the Environment (LIME), in the Earth and Environmental Systems Institute at the Pennsylvania State University. For protein immobilization, amine-functionalized agarose beads were purchased from Nanocs Inc. N-Succinimidyl 4-(maleimidomethyl) cyclohexane-1-carboxylate (SMCC) was purchased from Chem-Impex International, Inc. and used without further purification.

Circular dichroism (CD) spectroscopy. CD spectra of Hans-LanM were collected as previously described. In short, a Jasco J-1500 CD spectrometer was used to scan samples from 195 to 255 nm using the following settings: 1 nm bandwidth, 0.5 nm data pitch, 50 nm/min scan rate, 4 s average time. For all buffered metal and stoichiometric titrations, the cuvette contained 15 μM protein (monomer concentration). For stoichiometric titrations, the protein was diluted into Chelex-treated Buffer A (20 mM acetate, 100 mM KCl, pH 5.0) and titrated with 0.5-4.0 equiv. of each metal ion from a 1.5 mM solution in the same buffer.

Buffered metal solutions were prepared as previously described. EDTA was used as the chelator for titrations with Ca^{II}, La^{III}, and Nd^{III}; EGTA was used for Dy^{III}. Protein was added to 1 μM in the respective “high” and “low” metal solutions for each metal ion. These solutions were then combined to a final volume of 200 μL, in various ratios, to produce a range of free metal concentrations in the presence of Hans-LanM. These solutions were incubated overnight at 4° C. CD spectra were collected and the CD signal at 222 nm was plotted against the free metal concentration, yielding binding curves that were fitted using the Hill equation.

BioSAXS data acquisition. Data were collected at a wavelength of 1.54 Å on the home source at the Penn State X-Ray Crystallography Facility, with X-rays generated by a Rigaku MM007 rotating anode housed with the BioSAXS2000^{nano} Kratky camera system. The system includes OptiSAXS confocal max-flux optics that are designed specifically for SAXS and a HyPix-3000 Hybrid Photon Counting detector. The sample capillary-to-detector distance was 495.5 mm and was calibrated using silver behenate powder (The Gem Dugout, State College, PA). The useful q-space range (4π sin θ/λ, where 2θ is the scattering angle) was generally from qₘᵢₙ=0.008 Å⁻¹ to qₘₐₓ=0.3 Å⁻¹. The energy of the X-ray beam was 1.2 keV, with the Kratky block attenuation of 22% and a beam diameter of ˜100 m.

Protein samples were loaded using the autosampler onto a quartz capillary flow cell, mounted on a stage maintained at 22° C. and aligned in the X-ray beam. The sample cell and full X-ray flight path, including beam stop, were kept in vacuo (<1×10⁻³ torr) to eliminate air scatter. The Rigaku SAXSLAB software was programmed for automated data collection of each protein sample and matched buffers, with rigorous cleaning with 1 M NaOH prior to the start of the run and with water and ethanol between samples. Data reduction, including image integration and normalization, and background buffer data subtraction were also carried out using the SAXSLAB software. Six 10-min images from protein and buffer samples were collected and averaged after ensuring that no X-ray radiation damage had occurred. SAXS data overlays showed that there was no radiation decay over the 60 min of data collection. This was followed by reference buffer subtraction to obtain the raw SAXS scattering curve from only the protein.

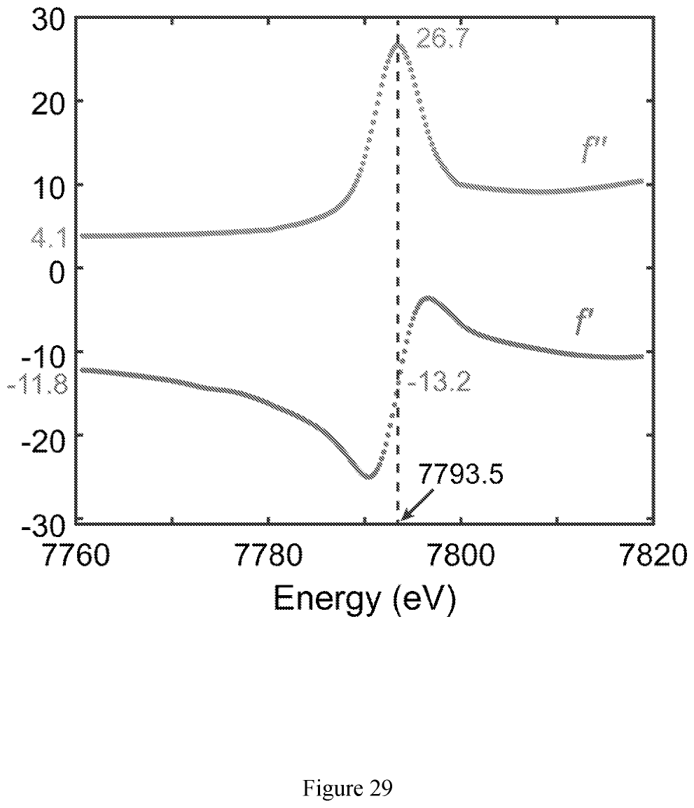

Dy-bound Hans-LanM structure determination. Anomalous scattering datasets were collected at National Cancer Institute Structural Biology Facility (GM/CA) beamline ID-D at the Advanced Photon Source (Argonne National Laboratory, Argonne, IL). The X-ray absorption spectrum was first measured (FIG. 29), and two energies, 7793.5 (L_{III} edge) and 7760 (pre-edge) eV, were chosen accordingly to maximize the differences in absorption (f″). The diffraction data at the two energies were collected alternately using inverse-beam geometry with 30-degree wedges to minimize radiation damage and subsequently processed with HKL2000.

The coordinates determined from high-resolution data were further refined against the new dataset collected at 7793.5 eV using phenix.refine to account for batch variations. As opposed to the high-resolution structure, no heavy element can be found in EF1 of chain D, while the remaining 13 dysprosium ions were retained. No other visible structural changes were observed. The anomalous difference map was prepared with phenix.maps, and the corresponding peak intensities (Table 10) were inspected via Coot. The large differences in anomalous peak intensities determined at the pre-edge and on-edge energies corresponding to Dy strongly indicate that the metals bound at EF hands are indeed Dy.

Maleimide functionalization of agarose beads. The maleimide functionalization of amine-functionalized agarose beads was described previously. Briefly, agarose microbeads (1.2 mL) were aliquoted into 5 mL Eppendorf tubes and preconditioned with pH 7.4 phosphate-buffered saline (PBS) and resuspended at a final volume of ˜1.7 mL (1.2 mL microbeads and 0.5 mL PBS supernatant). SMCC (0.15 g) powder was dissolved in 3.4 mL DMSO and combined with the microbeads. After 2.5 h incubation on a rocker mixer at room temperature, the modified agarose microbeads were washed with DMSO three times to remove unreacted SMCC and then washed with coupling buffer (50 mM HEPES, 50 mM KCl, pH 7.0) three times to remove DMSO. The maleimide-microbeads were then used for LanM immobilization within 2 h.

Breakthrough column experiments. Econo-Column glass chromatography columns (Bio-Rad; 5 cm×0.5 cm) were filled with MilliQ water (18.2 MΩ cm⁻¹) and LanM-microbeads were added gravimetrically. Columns were washed with 25 mM HCl, MilliQ water, and conditioned with Buffer D (10 mM homopiperazine-1,4-bis(2-ethanesulfonic acid), pH 5.0) before conducting breakthrough experiments. RE stock solutions were prepared by dissolving individual RE chloride salts in 1 mM HCl. The stock solutions were diluted in Buffer D. The RE solutions were pumped at 0.5 mL/min unless otherwise specified and the column effluent was collected in 1.0 mL aliquots. A washing step with 5 bed volumes of MilliQ water was included before performing desorption experiments with the stated concentrations of chelator or HCl. For single RE ion solutions, RE ion concentrations were quantified using the Arsenazo III assay. Specifically, 40 μL of sample was combined with 40 μL of 12.5 wt. % trichloroacetic acid (TCA) and then added to 120 μL of filtered 0.1 wt. % Arsenazo in 6.25 wt. % TCA. Absorbance at 652 nm was measured and compared to standards to determine the RE metal ion concentrations. The accuracy of the colorimetric assay was also confirmed by ICP-MS. For experiments with RE mixtures, the metal ion concentrations were determined by ICP-MS (Table 19, Table 16).

Table 1. ICP-MS analysis of metalated Hans-LanM samples used for SAXS or crystallography. Samples for SAXS analysis were incubated with 3 equiv. metal ion, and soluble protein was run on a S75 SEC column to remove aggregates. Preparation of samples for crystallography is described herein. The lower metal loading for the samples used for crystallography may relate to the additional spin concentration step and the third equivalent binding to a relatively weak site. The data are expressed as mean with s.d. from 3 technical replicates.

| | | Protein | Metal | |
|---|---|---|---|---|
| | Metal ion | concentration (μM) | concentration (μM) | Equivalents |
| SAXS | La | 120 | 336(8) | 2.8 |
| | Nd | 114 | 349(2) | 2.9 |
| | Dy | 128 | 269(16) | 2.4 |
| Crystallography | La | 1360 | 2310(60) | 1.7 |
| | Dy | 1450 | 2300(60) | 1.6 |

Table 2. Summary of fitted parameters for CD titrations of Hans-LanM with La^{III}, Nd^{III}, Dy^{III}, and Ca^{II}. Apparent K_{d} (K_{d,app}) values, Hill coefficients (n), and changes in molar ellipticity at 222 nm (Δ[Θ]) are reported as mean±s.e.m. for fits of data from 3 independent titrations. Conditions: 15 μM monomer concentration, 25° C., 20 mM acetate, 100 mM KCl, pH 5.0.

| | | | |
|---|---|---|---|
| | | | Δ[Θ] (deg cm² |
| Metal ion | K_{d, app} | n | dmol⁻¹ × 10⁻³) |

| | | | | |
|---|---|---|---|---|
| La^{III} | 68(7) | pM | 1.8(6) | −728(57) |
| Nd^{III} | 91(6) | pM | 2.0(2) | −722(20) |
| Dy^{III} | 2600(700) | pM | 1.3(4) | −449(56) |
| Ca^{II} | 60(10) | μM | 1.0(2) | −545(41) |

Table 3. Apparent molecular weights of RE^{III}-Hans-LanM complexes from analytical SEC (Superdex S75). Apparent MW values were derived from FIG. 8 and also plotted in FIG. 2a. Compare with SEC-MALS data in Table 17. Ionic radii are given for the 10-coordinate (La) and 9-coordinate (Nd-Ho) complexes previously described. See FIG. 8 legend for experimental details.

| | Ionic | Apparent MW |
|---|---|---|
| RE^{III} ion | radius (Å) | (SEC, kDa) |
| La | 1.16^{a} | 27.8 |
| Nd | 1.11^{b} | 27.8 |
| Sm | 1.08^{b} | 26.1 |
| Eu | 1.07^{b} | 25.5 |
| Gd | 1.05^{b} | 23.0 |
| Tb | 1.04^{b} | 21.3 |
| Dy | 1.03^{b} | 20.0 |
| Ho | 1.02^{b} | 17.9 |

^{a}CN = 10.
^{b}CN = 9.

Table 4. Data collection and refinement statistics for the X-ray structures of La- and Dy-Hans-LanM and Nd-Mex LanM. Statistics for the highest resolution shell are shown in parentheses.

| | La-Hans-LanM | Dy-Hans-LanM | Nd-Mex-LanM |
|---|---|---|---|
| Wavelength (Å) | 0.97857 | 0.97857 | 0.97857 |
| Resolution range | 40.11-1.8 | 24.84-1.4 | 23.13-1.01 |
| | (1.86-1.8) | (1.45-1.4) | (1.046-1.01) |
| Space group | P 2₁ | P 2₁ | P 2₁ 2₁ 2₁ |
| Unit cell | 40.107 108.658 44.697 | 56 54.234 65.275 | 24.099 44.398 82.316 |
| | 90 90.024 90 | 90 93.567 90 | 90 90 90 |
| Total reflections | 184147 | 566280 | 544124 |
| | (8072) | (43337) | (7577) |
| Non-anomalous | 27161 | 75504 | 43881 |
| unique reflections | (1945) | (6019) | (2296) |
| Multiplicity | 6.8 | 7.5 | 12.4 |
| | (4.2) | (7.2) | (3.3) |
| Completeness (%) | 72.2 | 97.5 | 91.0 |
| | (29.8) | (78.3) | (41.0) |
| Mean I/sigma(I) | 12.9 | 28.0 | 26.7 |
| | (1.3) | (2.3) | (2.4) |
| Wilson B-factor | 17.53 | 12.72 | 16.20 |
| R-meas | 0.133 | 0.069 | 0.076 |
| | (1.13) | (1.08) | (0.56) |
| R-pim | 0.052 | 0.025 | 0.021 |
| | (0.53) | (0.40) | (0.29) |
| CC½ | 0.995 | 0.997 | 0.986 |
| | (0.608) | (0.740) | (0.752) |
| Reflections used in refinement | 25533 | 75499 | 43022 |
| Reflections used for R-free | 1286 | 1985 | 1949 |
| R-work | 0.1898 | 0.1614 | 0.1453 |
| R-free | 0.2088 | 0.1809 | 0.1524 |
| Number of non-hydrogen | 3641 | 3979 | 964 |
| atoms | | | |
| macromolecules | 3336 | 3342 | 793 |
| ligands | 42 | 14 | 4 |
| solvent | 263 | 623 | 167 |
| Protein residues | 440 | 435 | 105 |
| RMS(bonds) | 0.003 | 0.007 | 0.006 |
| RMS(angles) | 0.48 | 0.80 | 0.81 |
| Ramachandran favored (%) | 96.53 | 99.06 | 100 |
| Ramachandran allowed (%) | 3.47 | 0.94 | 0.00 |
| Ramachandran outliers (%) | 0.00 | 0.00 | 0.00 |
| Rotamer outliers (%) | 0.00 | 0.57 | 0.00 |
| Clashscore | 0.60 | 2.23 | 3.79 |
| Average B-factor | 26.04 | 25.86 | 24.49 |
| macromolecules | 25.45 | 24.09 | 22.62 |
| ligands | 46.04 | 21.95 | 19.42 |
| solvent | 30.33 | 35.47 | 33.49 |

Table 5. SAXS structural parameters for Hans-LanM in presence of different metal ions. Data were collected on an in-house Rigaku BioSAXS2000^{nano}. Buffer conditions: 30 mM MOPS, 100 mM KCl, 500 glycerol, pH 7.0. The Porod volume analysis supports the conclusion that La and Nd complexes of Hans-LanM are largely dimeric and the Dy complex, only ˜2/3 the volume of the La and Nd complexes, is an equilibrium mixture of monomer and dimer. Note that there is less difference between the R_{g} values for the Dy and the La/Nd complexes than might be expected based on SEC-MALS (Table 17 suggests a ˜2-3 Å difference in hydrodynamic radius for La/Nd vs. Dy), because the protein concentrations used for SAXS are 5-fold higher than those for SEC-MALS, and therefore the population of Dy-bound dimer is substantially larger in the SAXS experiment. Nevertheless, these RE-dependent differences are within the uncertainty of the SAXS R_{g} values (see FIG. 15).

| | La-Hans- | Nd-Hans- | Dy-Hans- |
|---|---|---|---|
| | LanM | LanM | LanM |
| Concentration | 1.2 | 1.6 | 1.4 |
| prior to SEC- | | | |
| MALS (mg/mL) | | | |
| Guinier analysis | | | |
| I(0) (cm⁻¹) | 2.46 | 2.67 | 2.62 |
| R_{g} (Å) | 18.5 | 18.7 | 17.8 |
| q min (Å⁻¹) | 0.01 | 0.01 | 0.01 |
| q max (Å⁻¹) | 0.07 | 0.07 | 0.07 |
| P(r) analysis | | | |
| I(0) (cm⁻¹) | 2.46 | 2.67 | 2.62 |
| R_{g} (Å) | 18.5 | 18.7 | 17.8 |
| Dₘₐₓ (Å) | 59.0 | 60.6 | 55.6 |
| q range (Å⁻¹) | .01-.43 | .01-.42 | .01-.44 |
| Porod volume | 33261 | 29999 | 22599 |
| (Å³) | | | |

Table 6. Analyses using OLIGOMER and CRYSOL software suggest that the La and Nd complexes of Hans-LanM are nearly completely dimeric, but the Dy complex is a mixture of monomer and dimer. The OLIGOMER program fits an experimental SAXS scattering curve from a multicomponent mixture of proteins to find the volume fractions of each component in the mixture. CRYSOL evaluates the solution scattering from macromolecules with known atomic structure and fits it to experimental scattering curves. Both analyses were done with two possible states: the La-Hans-LanM crystallographic dimer and a crystallographic monomer. The analysis indicates that the volume fractions for the La- and Nd-Hans-LanM complexes are preferentially dimeric, likely close to the true distributions because of the availability of the crystallographic dimer model. OLIGOMER and CRYSOL analyses for Dy condition suggests an equilibrium between dimer and monomer.

| | La-Hans- | Nd-Hans- | Dy-Hans- |
|---|---|---|---|
| | LanM | LanM | LanM |
| OLIGOMER: | 0.94(1) | 0.88(1) | 0.55(5) |
| Volume fraction of | | | |
| dimer | | | |
| OLIGOMER: | 0.06(1) | 0.12(1) | 0.45(3) |
| Volume fraction of | | | |
| monomer | | | |
| CRYSOL: | 1.1 | 1.3 | 3.8 |
| Chi² fit to dimer | | | |
| CRYSOL: | 22.6 | 16.5 | 8.7 |
| Chi² fit to monomer | | | |

Table 7. Summary of fitted parameters for CD titrations of R100K-Hans-LanM with La^{III}, Nd^{III} and Dy^{III}. Apparent K_{d} (K_{d,app}) values, Hill coefficients (n), and changes in molar ellipticity at 222 nm (Δ[Θ]) are reported as mean±s.e.m. for fits of data from 2 (Nd, Dy) or 3 (La) independent titrations. Conditions: 15 μM monomer concentration, 25° C., 20 mM acetate, 100 mM KCl, pH 5.0.

| | | | Δ[Θ] (deg cm² |
|---|---|---|---|
| Metal ion | K_{d, app} (pM) | n | dmol⁻¹ × 10⁻³) |
| La | 120(10) | 1.9(2) | −829(28) |
| Nd | 99(7) | 2.8(5) | −697(32) |
| Dy | 2700(400) | 1.3(2) | −525(29) |

Table 8. SEC-MALS analysis of R100K-Hans LanM. The concentrations of the protein samples loaded to the column were 3 mg/mL (3.5 times more concentrated than wild-type Hans-LanM samples).

| | Peak retention | Molecular | | Hydrodynamic |
|---|---|---|---|---|
| | time (min) | weight (kDa) | Polydispersity | radius (nm) |
| Apo | 9.55 | 13.1 | 1.043 | 1.32 |
| | 10.1 | 12.4 | 1.000 | N.D.^{a} |
| La | 10.4 | 10.9 | 1.010 | 1.25 |
| Dy | 10.3 | 9.9 | 1.005 | 1.27 |

^{a}N.D., not determined

Table 9. Data collection statistics for the anomalous diffraction datasets collected on Dy-Hans-LanM at 7760.0 (pre-edge) and 7793.5 eV (L_{III} edge of Dy). Statistics for the highest resolution shell are shown in parentheses.

| | Dy-Hans LanM anomalous | |
|---|---|---|
| Wavelength (Å) | 1.59774 | 1.59087 |
| Resolution range | 43.56-2.22 | 43.56-2.21 |
| | (2.30-2.22) | (2.29-2.21) |
| Space group | P 2₁ | P 2₁ |
| Unit cell | 55.514 54.758 64.423 | 55.525 54.748 64.421 |
| | 90 94.2 90 | 90 94.2 90 |
| Total reflections | 228251 | 233868 |
| | (17496) | (18403) |
| Anomalous | 36946 | 37716 |
| unique reflections | (3392) | (3694) |
| Multiplicity | 6.2 | 6.2 |
| | (5.2) | (5.0) |
| Anomalous | 97.8 | 99.4 |
| completeness (%) | (90.4) | (96.8) |
| Mean I/sigma(I) | 21.9 | 20.7 |
| | (10.6) | (9.6) |
| Wilson B-factor | 26.33 | 25.89 |
| R-meas | 0.117 | 0.118 |
| | (0.252) | (0.240) |
| R-pim | 0.033 | 0.033 |
| | (0.081) | (0.076) |
| CC_{1/2} | 0.958 | 0.994 |
| | (0.608) | (0.981) |

Table 10. Anomalous peak heights for Dy-Hans-LanM at 7760.0 (pre-edge) and 7793.5 eV (L_{III} edge of Dy) in units of e/3. Interestingly, EF2 and EF3 exhibit the largest anomalous difference map peaks, perhaps reflecting the biochemical observation of only two high-affinity sites in this complex (FIG. 1d, FIG. 6). N/A: not applicable. *No heavy elements are found in EF1 of chain D in this crystal.

| Crystal 1 | Chain A | | Chain B | | Chain C | | Chain D | |
|---|---|---|---|---|---|---|---|---|
| Energy (eV) | 7793.5 | 7760.0 | 7793.5 | 7760.0 | 7793.5 | 7760.0 | 7793.5 | 7760.0 |
| EF1 | 0.699 | 0.109 | N/A | N/A | N/A | N/A | N/A* | N/A* |
| EF2 | 1.513 | 0.224 | 1.737 | 0.269 | 1.692 | 0.265 | 1.564 | 0.226 |
| EF3 | 1.532 | 0.246 | 1.526 | 0.246 | 1.711 | 0.275 | 1.846 | 0.278 |
| EF4 | 0.564 | 0.124 | 0.379 | 0.108 | 0.583 | 0.146 | 0.962 | 0.176 |

Table 11. Concentrations of citrate and malonate required for 50% desorption of La^{III}, Nd^{III}, Dy^{III} from Hans-LanM and R100K-Hans LanM. See FIG. 4a and FIG. 54. Trp emission intensity changes in Hans-LanM were monitored at 333 nm. Initial conditions: 20 μM protein, 4 μM RE, 20 mM acetate, 100 mM KCl, pH 5.0, into which increasing concentrations of citrate or malonate were titrated. Data represent mean s.e.m. (in parentheses) for fits of data from 3 independent titrations.

| | |
|---|---|
| [citrate]_{1/2} (mM) | [malonate]_{1/2} (mM) |

Hans-LanM

| | | |
|---|---|---|
| La | 18(3) | — |
| Nd | 7.5(8) | >350^{a} |
| Dy | 1.0(1) | 144(17) |

R100K-Hans-LanM

| | | |
|---|---|---|
| La | 12(1) | — |
| Nd | 5.6(6) | >350 |
| Dy | 1.0(1) | 128(11) |

^{a}350 mM was the highest concentration tested. See FIG. 4b.

Table 12. Distribution factors (D, in bold) and separation factors (SF) for selected REs for immobilized Mex-LanM. D values represent distribution of a particular metal ion between LanM and the solution in the multi-element equilibration experiment described in the main text and Materials and Methods (“Batch experiment to determine separation factors”), in which a 5 mL feed solution of equimolar REs from La to Dy (3 mM total REs, 15 μmol total, pH 5.0) reaches equilibrium with 1 mL immobilized LanM microbeads (capacity: 5.8 μmol for Mex-LanM, 4.1 μmol for Hans-LanM, 4.7 μmol for R100K-Hans-LanM). Larger D values indicate preferential adsorption to LanM. Separation factors as a function of RE identity were calculated as Dₘₑₜₐₗ₍ₜₒₚ₎/D_{metal(left)}. An SF of 1.0 indicates no selectivity for intra-RE separation, while SFs much greater (or less) than 1 indicate preference for a particular ion over another. See FIG. 4c for a plot of the common logarithm of D values for each metal ion. Uncertainties are shown in parentheses, where the number is parentheses is the uncertainty in the significant digits immediately preceding: e.g., 0.86(1) means 0.86±0.01, and 2.15(32) means 2.15±0.32. The uncertainties are larger for HREs like Dy, especially with Hans-LanM and R100K-Hans-LanM, because the weaker binding to LanM means quantities of REs adsorbed to the column are very low compared to the LREs. Uncertainties for D values are standard deviations from three independent column runs. Uncertainties for SF values were calculated by error propagation of the corresponding D values.

| | | La | Ce | Pr | Nd | Sm | Eu | Gd | Tb | Dy |
|---|---|---|---|---|---|---|---|---|---|---|
| | D | 0.86(1) | 1.52(3) | 1.90(4) | 2.15(32) | 2.19(5) | 1.68(2) | 0.93(2) | 0.65(1) | 0.41(1) |
| La | 0.86(1) | 1 | 1.77(5) | 2.21(6) | 2.50(37) | 2.55(8) | 1.95(4) | 1.08(3) | 0.75(2) | 0.48(1) |
| Ce | 1.52(3) | 0.57(2) | 1 | 1.25(4) | 1.42(21) | 1.44(5) | 1.10(3) | 0.61(2) | 0.43(1) | 0.27(1) |
| Pr | 1.90(4) | 0.45(1) | 0.80(2) | 1 | 1.13(17) | 1.15(4) | 0.88(2) | 0.49(1) | 0.34(1) | 0.22(1) |
| Nd | 2.15(32) | 0.40(6) | 0.71(11) | 0.88(13) | 1 | 1.02(15) | 0.78(12) | 0.43(6) | 0.30(4) | 0.19(3) |
| Sm | 2.19(5) | 0.39(1) | 0.69(2) | 0.87(3) | 0.98(15) | 1 | 0.76(2) | 0.42(1) | 0.30(1) | 0.19(1) |
| Eu | 1.68(2) | 0.51(1) | 0.91(2) | 1.13(3) | 1.28(19) | 1.31(4) | 1 | 0.55(1) | 0.39(1) | 0.25(1) |
| Gd | 0.93(2) | 0.93(3) | 1.64(5) | 2.05(6) | 2.32(35) | 2.37(8) | 1.81(4) | 1 | 0.70(2) | 0.44(1) |
| Tb | 0.65(1) | 1.33(4) | 2.34(7) | 2.93(8) | 3.31(49) | 3.38(11) | 2.58(6) | 1.43(4) | 1 | 0.63(2) |
| Dy | 0.41(1) | 2.09(6) | 3.70(12) | 4.63(14) | 5.23(78) | 5.33(19) | 4.08(12) | 2.25(7) | 1.58(5) | 1 |

Table 13. Distribution factors (D) and separation factors (SFs) for selected REs for immobilized Hans-LanM. See legend for Table 12 for full details.

| | | La | Ce | Pr | Nd | Sm | Eu | Gd | Tb | Dy |
|---|---|---|---|---|---|---|---|---|---|---|
| | D | 0.86(3) | 1.54(4) | 1.71(5) | 1.44(4) | 0.99(4) | 0.64(3) | 0.34(2) | 0.22(2) | 0.15(2) |
| La | 0.86(3) | 1 | 1.79(9) | 1.98(10) | 1.67(8) | 1.14(6) | 0.75(5) | 0.39(3) | 0.25(2) | 0.17(2) |
| Ce | 1.54(4) | 0.56(3) | 1 | 1.11(5) | 0.93(4) | 0.64(3) | 0.42(2) | 0.22(1) | 0.14(1) | 0.10(1) |
| Pr | 1.71(5) | 0.51(3) | 0.90(4) | 1 | 0.84(4) | 0.58(3) | 0.38(2) | 0.20(1) | 0.13(1) | 0.09(1) |
| Nd | 1.44(4) | 0.60(3) | 1.07(5) | 1.19(5) | 1 | 0.69(3) | 0.45(3) | 0.23(2) | 0.15(1) | 0.10(1) |
| Sm | 0.99(4) | 0.87(5) | 1.56(7) | 1.73(8) | 1.46(7) | 1 | 0.65(4) | 0.34(2) | 0.22(2) | 0.15(2) |
| Eu | 0.64(3) | 1.34(9) | 2.39(14) | 2.65(16) | 2.23(13) | 1.53(9) | 1 | 0.52(4) | 0.34(3) | 0.23(3) |
| Gd | 0.34(2) | 2.56(19) | 4.58(31) | 5.07(35) | 4.26(30) | 2.93(21) | 1.91(15) | 1 | 0.65(7) | 0.44(6) |
| Tb | 0.22(2) | 3.93(38) | 7.02(65) | 7.78(73) | 6.54(61) | 4.50(43) | 2.94(30) | 1.53(17) | 1 | 0.67(10) |
| Dy | 0.15(2) | 5.86(70) | 10.49(1.21) | 11.61(1.34) | 9.77(1.13) | 6.71(79) | 4.39(54) | 2.29(29) | 1.49(21) | 1 |

Table 14. Distribution factors (D) and separation factors (SFs) for selected REs for immobilized R100K-Hans-LanM. See legend for Table 12 for full details.

| | | La | Ce | Pr | Nd | Sm | Eu | Gd | Tb | Dy |
|---|---|---|---|---|---|---|---|---|---|---|
| | D | 1.18(4) | 2.14(8) | 2.38(9) | 1.92(8) | 1.22(8) | 0.88(8) | 0.41(4) | 0.25(3) | 0.14(2) |
| La | 1.18(4) | 1 | 1.82(1) | 2.02(11) | 1.63(9) | 1.04(8) | 0.75(7) | 0.35(4) | 0.21(3) | 0.12(2) |
| Ce | 2.14(8) | 0.55(3) | 1 | 1.11(6) | 0.90(5) | 0.57(4) | 0.41(4) | 0.19(2) | 0.12(2) | 0.07(1) |
| Pr | 2.38(9) | 0.49(3) | 0.90(5) | 1 | 0.81(5) | 0.52(4) | 0.37(4) | 0.17(2) | 0.10(1) | 0.06(1) |
| Nd | 1.92(8) | 0.61(3) | 1.12(6) | 1.24(7) | 1 | 0.64(5) | 0.46(4) | 0.22(2) | 0.13(2) | 0.07(1) |
| Sm | 1.22(8) | 0.96(7) | 1.75(13) | 1.94(14) | 1.56(12) | 1 | 0.72(8) | 0.34(4) | 0.20(3) | 0.12(2) |
| Eu | 0.88(8) | 1.34(13) | 2.43(23) | 2.70(26) | 2.17(21) | 1.39(15) | 1 | 0.47(6) | 0.28(5) | 0.16(3) |
| Gd | 0.41(4) | 2.84(31) | 5.16(56) | 5.73(62) | 4.62(51) | 2.95(35) | 2.12(28) | 1 | 0.60(10) | 0.34(7) |
| Tb | 0.25(3) | 4.71(65) | 8.57(1.19) | 9.52(1.32) | 7.68(1.07) | 4.91(72) | 3.53(56) | 1.66(28) | 1 | 0.57(12) |
| Dy | 0.14(2) | 8.34(1.50) | 15.16(2.74) | 16.84(3.04) | 13.58(2.46) | 8.68(1.62) | 6.24(1.23) | 2.94(60) | 1.77(39) | 1 |

Table 15. Amino acid and DNA sequences of constructs used in this study. The first residue of the cytosolically expressed Hans-LanM proteins (after cleavage of the N-terminal Met) is A24, the predicted signal peptide cleavage site according to SignalP 6.0; all residues are numbered based on the full-length sequence.

| Construct | Protein or DNA sequence |
|---|---|
| Hans-LanM (full sequence, | MKLSLKAGAA ITAFVFAASP VLAASGADAL KALNKDNDDS |
| signal peptide underlined) | LEIAEVIHAG ATTFTAINPD GDTTLESGET KGRLTEKDWA |
| | RANKDGDQTL EMDEWLKILR TRFKRADANK DGKLTAAELD |
| | SKAGQGVLVM IMK (SEQ ID NO:1) |
| Hans-LanM (lacking signal | MASGADAL KALNKDNDDS LEIAEVIHAG ATTFTAINPD |
| peptide, as expressed in this | GDTTLESGET KGRLTEKDWA RANKDGDQTL EMDEWLKILR |
| study) | TRFKRADANK DGKLTAAELD SKAGQGVLVM IMK (SEQ ID |
| | NO:2) |
| R100K-Hans-LanM (mutation | MASGADAL KALNKDNDDS LEIAEVIHAG ATTFTAINPD |
| underlined) | GDTTLESGET KGRLTEKDWA RANKDGDQTL EMDEWLKILK |
| | TRFKRADANK DGKLTAAELD SKAGQGVLVM IMK |
| | (SEQ ID NO : 4) |
| Hans-LanM-Cys (for | MASGADAL KALNKDNDDS LEIAEVIHAG ATTFTAINPD |
| immobilization) | GDTTLESGET KGRLTEKDWA RANKDGDQTL EMDEWLKILR |
| | TRFKRADANK DGKLTAAELD SKAGQGVLVM IMKGSGC |
| | (SEQ ID NO : 5) |
| R100K-Hans-LanM-Cys (for | MASGADAL KALNKDNDDS LEIAEVIHAG ATTFTAINPD |
| immobilization) | GDTTLESGET KGRLTEKDWA RANKDGDQTL EMDEWLKILK |
| | TRFKRADANK DGKLTAAELD SKAGQGVLVM IMKGSGC |
| | (SEQ ID NO : 6) |
| Hans-LanM codon-optimized | ATGGCAAGTGGCGCGGATGCTTTGAAGGCGCTTAACAAAGACAAT |
| DNA sequence for this study | GACGATTCGCTGGAAATTGCAGAGGTAATCCACGCAGGCGCAACT |
| (EF-hands underlined) | ACGTTCACGGCAATCAACCCGGACGGAGACACAACTTTGGAGAGC |
| | GGAGAGACGAAAGGACGCTTGACAGAAAAGGATTGGGCTAGAGCT |
| | AATAAAGACGGGGACCAGACGTTGGAAATGGACGAATGGCTGAAG |
| | ATCCTGCGTACTAGATTTAAAAGAGCCGATGCTAATAAGGATGGC |
| | AAATTAACGGCTGCGGAGTTGGATTCCAAAGCGGGGCAAGGGGTA |
| | TTGGTCATGATCATGAAATGA (SEQ ID NO : 45) |
| R100K-Hans-LanM DNA | ATGGCAAGTGGCGCGGATGCTTTGAAGGCGCTTAACAAAGACAAT |
| sequence (mutation underlined) | GACGATTCGCTGGAAATTGCAGAGGTAATCCACGCAGGCGCAACT |
| | ACGTTCACGGCAATCAACCCGGACGGAGACACAACTTTGGAGAGC |
| | GGAGAGACGAAAGGACGCTTGACAGAAAAGGATTGGGCTAGAGCT |
| | AATAAAGACGGGGACCAGACGTTGGAAATGGACGAATGGCTGAAG |
| | ATCCTGAAAACTAGATTTAAAAGAGCCGATGCTAATAAGGATGGC |
| | AAATTAACGGCTGCGGAGTTGGATTCCAAAGCGGGGCAAGGGGTA |
| | TTGGTCATGATCATGAAATGA (SEQ ID NO: 46) |
| Hans-LanM-Cys DNA sequence | ATGGCAAGTGGCGCGGATGCTTTGAAGGCGCTTAACAAAGACAAT |
| (GSGC underlined) | GACGATTCGCTGGAAATTGCAGAGGTAATCCACGCAGGCGCAACT |
| | ACGTTCACGGCAATCAACCCGGACGGAGACACAACTTTGGAGAGC |
| | GGAGAGACGAAAGGACGCTTGACAGAAAAGGATTGGGCTAGAGCT |
| | AATAAAGACGGGGACCAGACGTTGGAAATGGACGAATGGCTGAAG |
| | ATCCTGCGTACTAGATTTAAAAGAGCCGATGCTAATAAGGATGGC |
| | AAATTAACGGCTGCGGAGTTGGATTCCAAAGCGGGGCAAGGGGTA |
| | TTGGTCATGATCATGAAAGGCAGCGGCTGCTGA (SEQ ID NO: 47) |
| R100K-Hans-LanM-Cys DNA | ATGGCAAGTGGCGCGGATGCTTTGAAGGCGCTTAACAAAGACAAT |
| sequence | GACGATTCGCTGGAAATTGCAGAGGTAATCCACGCAGGCGCAACT |
| | ACGTTCACGGCAATCAACCCGGACGGAGACACAACTTTGGAGAGC |
| | GGAGAGACGAAAGGACGCTTGACAGAAAAGGATTGGGCTAGAGCT |
| | AATAAAGACGGGGACCAGACGTTGGAAATGGACGAATGGCTGAAG |
| | ATCCTGAAAACTAGATTTAAAAGAGCCGATGCTAATAAGGATGGC |
| | AAATTAACGGCTGCGGAGTTGGATTCCAAAGCGGGGCAAGGGGTA |
| | TTGGTCATGATCATGAAAGGCAGCGGCTGCTGA (SEQ IDNO: 48) |

Table 16. Metal ion concentrations in the synthetic feed solution for on-column Nd/Dy separation in FIG. 4d and FIG. 55. Concentrations were determined by ICP-MS analysis.

| | Mean | Standard | Coefficient of |
|---|---|---|---|
| | concentration (μM) | deviation | variation (%) |
| Dy | 18.5 | 0.9 | 4.4 |
| Nd | 344.2 | 4.4 | 5.0 |

### Example 2

This Example describes use of the peptides/proteins of the present disclosure.

The structures of Hans- and Mex-LanM, in addition to the appreciation for the mechanism of dimerization of Hans-LanM, suggest several routes for increasing metal-binding selectivity, stoichiometry, and cooperativity, as well as modulating K_{dimer} overall such that the dimerization equilibria are poised in the higher concentration regime more suited to an industrial process rather than in the cell.

Increased selectivity would take advantage of the additional stabilization of the metal-protein complexes imparted by the dimerization by identifying points in the lanthanide series where there are large changes in dimerization propensity—where the complex with one lanthanide(III) ion (e.g. Tb) stabilizes the dimer to a substantially greater extent than the next lanthanide ion (e.g. Dy). Because the position of the dimerization equilibria will depend on the concentration of the monomers, it is important to be able to tune K_{dimer} to reflect the protein concentration in the intended use condition (in most cases, to weaken dimerization affinity).

Increased stoichiometry would make for more economical REE binding (better atom economy), while increased cooperativity would allow for sharper desorption profiles and thus potentially better separations.

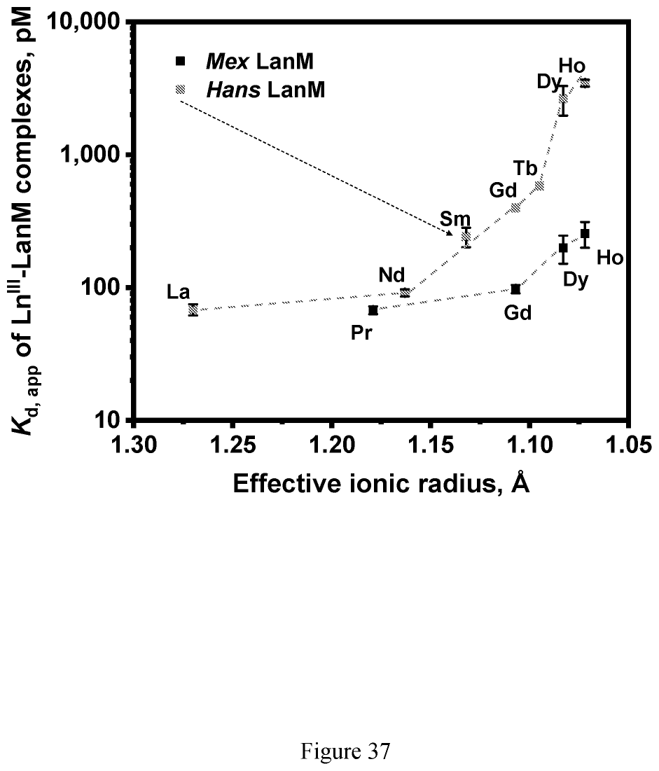

Increasing separation potential using Hans-LanM. The response of Hans-LanM to Sm(III), Gd(III), Tb(III), and Ho(III) was determined using CD spectroscopy to fill out the selectivity plot in FIG. 1e and Table 2 (FIG. 37, Table 20). The experimental conditions were the same as described above (15 μM Hans-LanM, in 30 mM acetate, 100 mM KCl, 10 mM EGTA, 0-10 mM Ln(III), pH 5.0).

Table 20. Expansion of Table 2 to include Sm, Gd, Tb, and Ho. Values reported with uncertainties were performed at least twice.

| | | | |
|---|---|---|---|
| | | | Δ[Θ] (deg cm² |
| Metal ion | K_{d, app} | n | dmol⁻¹ × 10⁻³) |

| | | | | |
|---|---|---|---|---|
| La^{III} | 68(7) | pM | 1.8(6) | −728(57) |
| Nd^{III} | 91(6) | pM | 2.0(2) | −722(20) |
| Sm^{III} | 240 (40) | pM | 1.3(2) | −575(89) |
| Gd^{III} | 397 | pM | 1.97 | −512 |
| Tb^{III} | 583 | pM | 1.39 | −476 |
| Dy^{III} | 2600(700) | pM | 1.3(4) | −449(56) |
| Ho^{III} | 3500(210) | pM | 1.2(1) | −481(76) |
| Ca^{II} | 60(10) | μM | 1.0(2) | −545(41) |

These data follow the general trend noted above that the magnitude of the protein's response to Dy(III) (Δ[Θ]) is smaller than to La(III) and Nd(III); this decrease in magnitude occurs roughly at Sm(III). This also correlates with a sudden drop in apparent K_{d} (2.5-3 fold) that occurs between Nd(III) and Sm(III). A second sudden dropoff in apparent K_{d} (5-fold) occurs between Tb(III) and Dy(III), which would be an industrially important separation. It is not clear at present whether the Hill coefficients of ˜2 for La(III)- and Nd(III)-bound Hans-LanM (Table 2) reflect metal-binding to EF3 and dimerization of two monomers or EF2/3 from a single monomer. One interpretation of the lower cooperativity of Dy(III) (and other MREE-HREE) binding is that indeed it is the metal-dependent dimerization that leads to cooperativity. These results motivate exploration of the use of dimerization equilibria of variants of this protein to carry out this separation.

In order to do this, it is important to understand the interactions contributing to dimer stabilization. The crystal structures of Hans-LanM suggest that the R100-centered hydrogen bonding network is a key contributor, but other interactions at the interface may also play a role (e.g., M92, 143, 147, L96). This suggestion is supported by ITC analysis of the R100K variant (Table 21). While the presence of glycerol in these experiments may somewhat affect the quantitative thermodynamic values, the data show that, in R100K, the K_{dimer} for the La(III) complex has increased substantially, from <0.4 μM to 14 μM, whereas the K_{dimer} values for apo and Dy(III)-bound R100K are similar to the wild-type protein. This further clarifies that R100-dependent dimerization selectively enhances the stability of the La-Nd complexes.

Table 21. Thermodynamics parameters for WT and R100K Hans-LanM, obtained by fitting ITC thermograms to the dimer dissociation model. These experiments were carried out similarly to those shown in Table 18, except that 5% glycerol was present in the buffer.

| | Apo | | La | | Dy | |
|---|---|---|---|---|---|---|
| | wt | R100K | wt | R100K | wt | R100K |
| Kd | 100(60) | 82(23) | nd | 14(4) | 40(1) | 54(4) |
| ΔH | 24(2) | 16(0.2) | nd | −19(3) | −5(1) | −16(0.3) |
| ΔG | 6.3(1.6) | 6(0.2) | nd | 7(0.2) | 6.1(0.1) | 5.9(0.1) |
| ΔS | 61(9) | 37(5) | nd | −84(11) | −36(2) | −73(1) |

Thus, for dimerization equilibria to come into play at millimolar protein concentrations (desirable for separations), some of the other interactions contributing to dimerization should be removed. To accomplish this, some of the following variants (individually and potentially in combination) will be pursued:
M92L, M92A, or M92D: A leucine is present at this position in LanM_012, which does not appear to dimerize significantly in the absence of REEs (see below). These mutations could make the middle of the dimer interface less hydrophobic and therefore decrease residual part of Hans-LanM's dimerization evident in the ITC and other studies.
A44N, A44S, or A44T: These mutations are hypothesized to add a hydrogen bond between this residue in one monomer and, for example, the T7 residue in either EF2 or EF3 of the adjacent monomer. If the interaction occurs with EF2, this could allow for specific interactions with both EF2 and EF3 in the dimer, tying dimerization to occupancy of both EF2 and EF3, and perhaps enhancing cooperativity and the affinity drop-offs observed in FIG. 37.
D93N, D93A, or D93E: D93N or D93A mutations would likely weaken the interaction of R100 with the adjacent monomer, such that only/primarily hydrogen bonding to the metal ligands contributes to dimerization in this region of the protein. D93E may alter the selectivity of the REE-induced dimerization by altering the hydrogen bonding network such that the REE for which dimerization is tightest is changed.

For the sake of being able to immobilize a version of Hans-LanM that is capable of dimerization on-column, two monomers can be connected using a linker of appropriate length and flexibility, such as a polypeptide sequence. Examples of potential constructs are below (the first is likely too short to support intramolecular dimerization; the linker sequences are underlined, and the rest of the sequence is the Hans-LanM sequence):

>HansR100K-L1
(SEQ ID NO: 16)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSASGADALKALNKDNDDSL
EIAEVIHAGATTFTAINPDGDTTLESGETKGRLTEKDWARANKDGDQTL
EMDEWLKILKTRFKRADANKDGKLTAAELDSKAGQGVLVMIMK
>HansR100K-L2
(SEQ ID NO: 17)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSASGADA
LKALNKDNDDSLEIAEVIHAGATTFTAINPDGDTTLESGETKGRLTEKD
WARANKDGDQTLEMDEWLKILKTRFKRADANKDGKLTAAELDSKAGQGV
LVMIMK
>HansR100K-L3
(SEQ ID NO: 18)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSGGSGGS
ASGADALKALNKDNDDSLEIAEVIHAGATTFTAINPDGDTTLESGETKG
RLTEKDWARANKDGDQTLEMDEWLKILKTRFKRADANKDGKLTAAELDS
KAGQGVLVMIMK
>HansR100K-L4
(SEQ ID NO: 19)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGGSGGSGGSGGSGGSGGSGGSGGSGGSGGSGGSGGS
GGSGGSASGADALKALNKDNDDSLEIAEVIHAGATTFTAINPDGDTTLE
SGETKGRLTEKDWARANKDGDQTLEMDEWLKILKTRFKRADANKDGKLT
AAELDSKAGQGVLVMIMK
>HansR100K-L5
(SEQ ID NO: 20)
MASGADALKALNKDNDDSLEIAEVIHAGATTFTAINPDGDTTLESGETK
GRLTEKDWARANKDGDQTLEMDEWLKILKTRFKRADANKDGKLTAAELD
SKAGQGVLVMIMKGSGGSGAEAAAKEAAAKAGGSGGSAEAAAKEAAAKA
GSGGSGASGADALKALNKDNDDSLEIAEVIHAGATTFTAINPDGDTTLE
SGETKGRLTEKDWARANKDGDQTLEMDEWLKILKTRFKRADANKDGKLT
AAELDSKAGQGVLVMIMK.

The dimerization variants and the linker variants could be combined for separation experiments on column or in solution at higher protein concentrations.

Metal-binding stoichiometry of M. extorquens LanM. Previous work on M extorquens LanM (Featherston et al. JACS 2021; Mattocks et al. Chem. Sci. 2022; Dong et al., ACS Cent. Sci. 2021) showed that the third binding equivalent, assigned to EF1, is kinetically more labile than the prior two, which manifests as a binding stoichiometry of 2 at low pH and on-column conditions. For example, at pH 5, the apparent K_{d} of the conformational change of EF-hands 2 and 3 in response to Nd³⁺ is 21 μM, while that of EF1 is 4.1 nM (Mattocks et al., Chem. Sci. 2022). Therefore, we sought an approach to bring the affinity of EF1 in line with EF2/3, thereby increasing the stoichiometry to (at least) 3. The general approach is to install unique hydrogen bonds in the folded state of the protein between EF hands, which could enhance affinity.

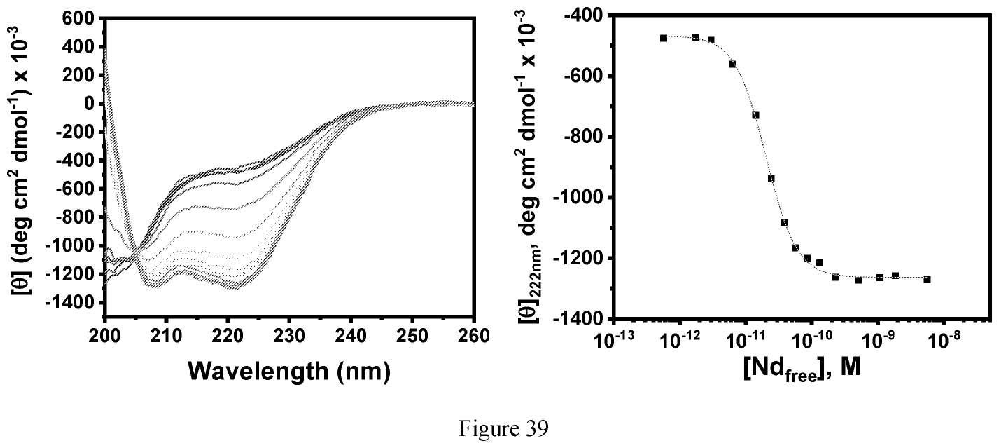

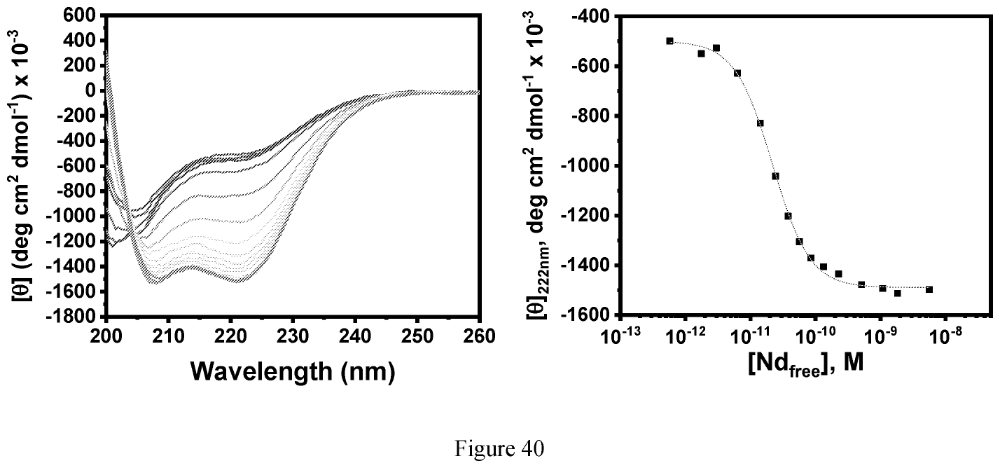

The crystal structure of Nd-Mex-LanM shows a hydrogen bond between D56 (residue at the “−3” position of EF2, with the minus sign denoting its being prior to the loop) and K93 (10^{th} residue of EF3). Meanwhile, the equivalent interaction for the EF1/4 pair (A32, −3 of EF1, and A117, 10^{th} residue of EF4) is absent. We hypothesized that installing the equivalent hydrogen bond in this pair might contribute to stabilization of EF1. The substitutions A117K and A117R were made in the background of A32D, and these two proteins (A32D/A117K and A32D/A177R) were expressed and purified from E. coli as for wild-type Mex-LanM. The proteins' responses to Nd³⁺ were characterized by CD at pH 5.0 for direct comparison with the wild-type data (Mattocks et al., Chem. Sci. 2022) (FIGS. 39 and 40).

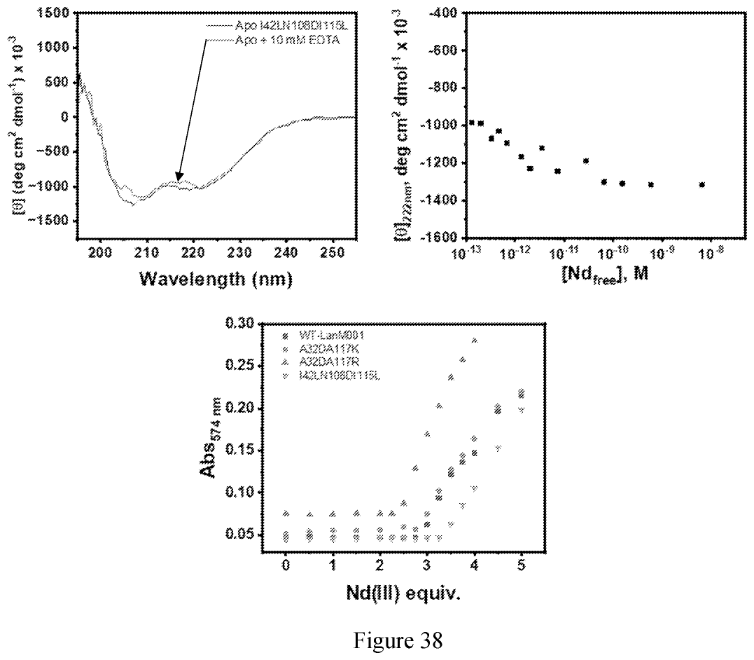

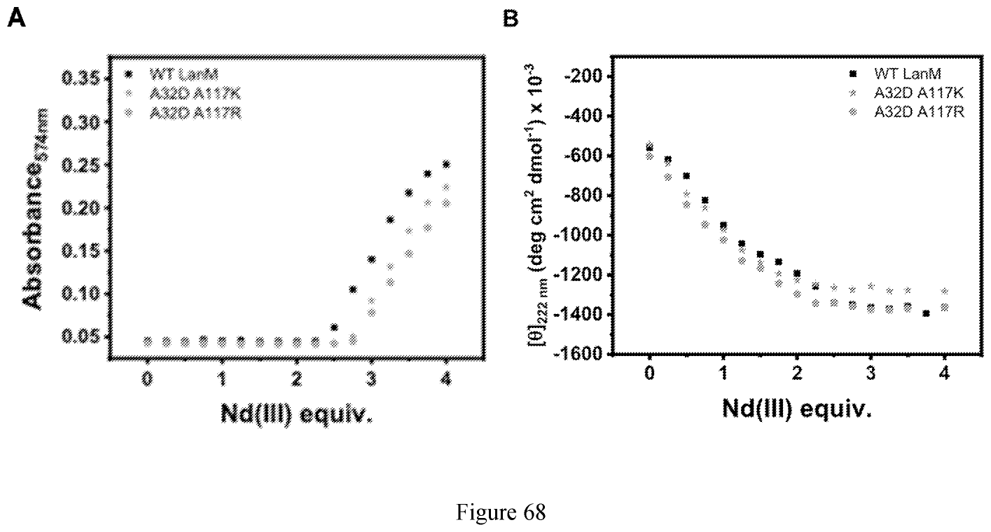

While both sets of substitutions decrease the apparent K_{d}, the A32D/A117K variant appears to be better behaved. In A32D/A117R, the overall response (FIG. 40, right) is slightly asymmetric, suggesting that EF1 exhibits an improved response versus wild-type (see FIG. 2), but not as similar to EF2/3 as in A32D/A117K. In A32D/A117K, it is likely that EF1 is not cooperatively responding with EF2/3, based on the value of the Hill coefficient; it is not yet clear whether the improved response will be sufficient to increase binding stoichiometry, or whether this site will still be kinetically less stable than EF2/3. While initial xylenol orange competition experiments suggested that A32D/A117R is somewhat destabilizing on overall equivalents of metal binding, whereas A32D/A117K is similar to WT and I42L/N108D/I115L is slightly stabilizing relative to WT (FIG. 38, bottom), replicates of these results suggest that A32D/A117R and A32D/A117K perform similarly, and somewhat better than WT (see below and FIG. 68). The binding stoichiometry will be tested at different pHs using spin columns to separate bound vs. unbound metal ions, and it will also be tested using a Cys-modified version immobilized to agarose beads, as previously described. The combination of the best variants (e.g., A32D/A117K and I42L/N108D/I115L) will also be tested similarly.

The observation of EF4 occupied with Nd³⁺ in the crystal structure with nearly identical EF-loop structure (except for the different hydrogen bonding pattern induced by the N1 residue) suggests that the low stability of this site may result from the lack of a conformational change induced by metal binding. The structure reveals that the helix between EF3 and EF4 is slightly unwound starting at A98, which may result from occupancy of EF4. Adding a helical break in this region (i.e., substituting A98, A99, V100, or possibly A102 with Gly) could both optimize the structure of EF4 and disconnect it from EF3, perhaps providing enough conformational change to increase affinity. Similarly, there is a small break in the helix between EF1 and EF2 at G51, which may explain why EF1 behaves largely independently of EF2. A G51 Å substitution may link EF1 to EF2/3, possibly increasing the Hill coefficient to 3. A method to test the effects of these mutations is the fluorescence of Y96 at pH around 5, which decreases with addition of the first two binding equivalents but then increases upon addition of the third, as if the third equivalent is slightly destabilizing to the folded structure of the protein. A mutation that retains the lower Tyr fluorescence emission upon addition of the third equivalent will be explored.

The combination of these mutations to these two helices, along with the A32D/A117K and/or I42L/N108D/I115L variants described above, may enable a tight-binding stoichiometry of up to 4, as well as connection of all 4 EF hands into a single cooperative unit (n up to 4), which may significantly improve separation of adjacent REs. If this is not possible, activation of EF4 and cooperative binding with EF1 may yield two halves of the protein, each with n=2, which would still be a significant improvement in protein performance.

Conceptually related to linking EF1 and EF4 together via a hydrogen bond is use of a disulfide. As described in FIG. 27 above, natural LanMs exist with this property, and the metal-binding properties of these proteins will be investigated. An example of such a protein has the following sequence (after cleavage of the putative signal sequence; the EF hands are underlined):

>LanM 011
(SEQ ID NO: 49)
GHHNCKAEMAYLNPDHDGTIDWREARRAAVRLFHKLDPDHDGTLDMKEV
RGRVGILSFARFNPDRDGKLDKHEWLALVKHRFHRANPDKDGTIDCREL
HSLAGRKLERVLM

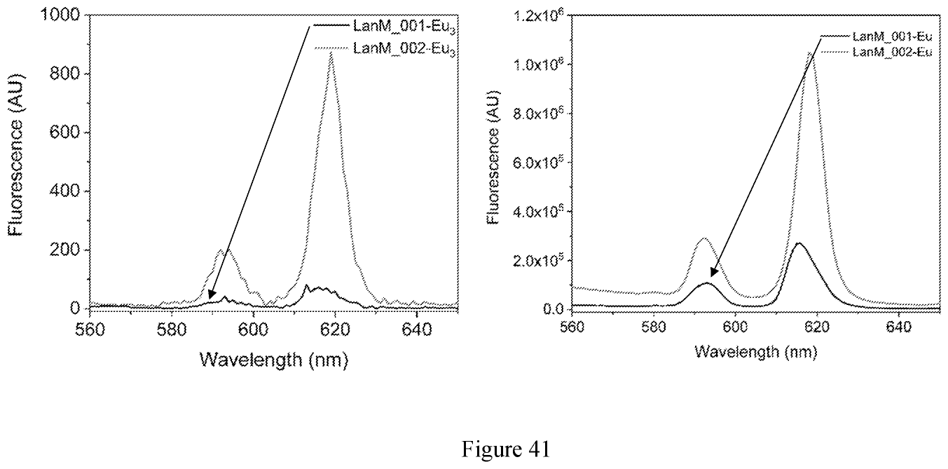

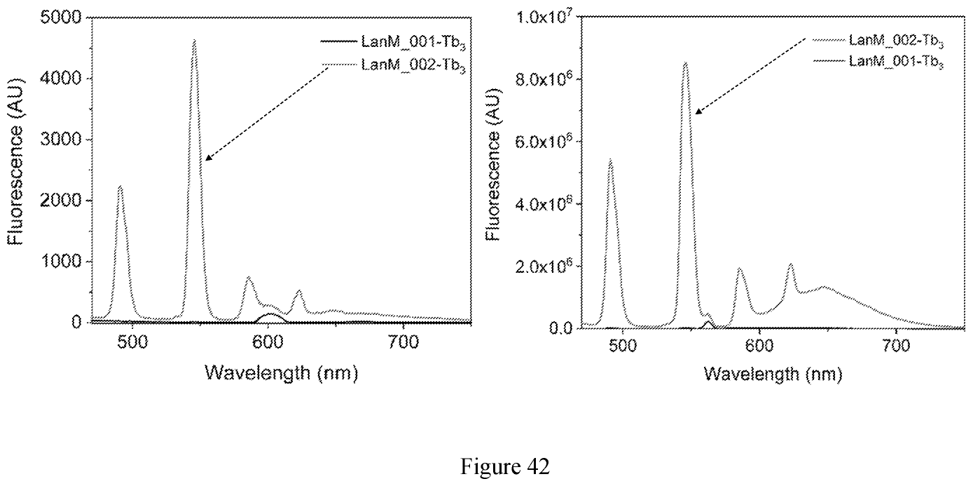

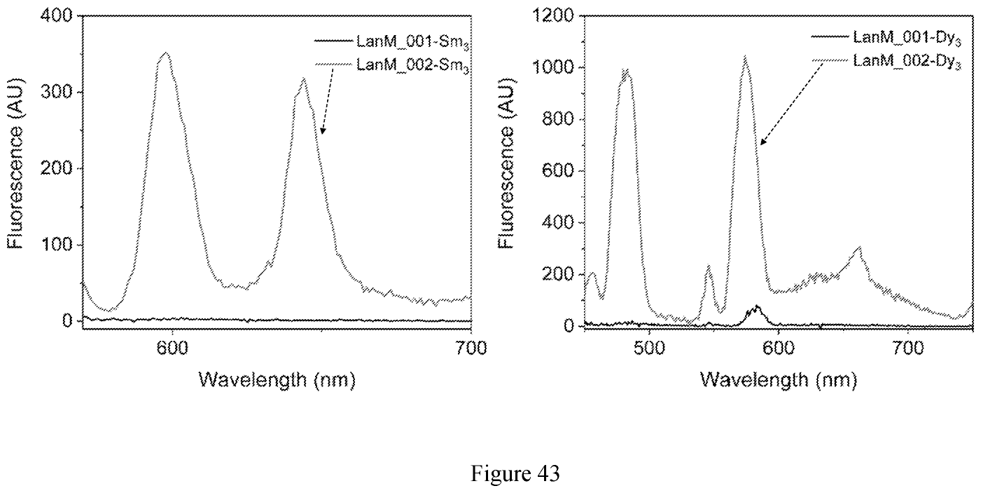

LanM_002 is a stronger sensitizer for Sm^{III}, Eu^{III}, Tb^{III}, and Dy^{III} luminescence than LanM_001. The observation that Hans-LanM (LanM_002) has zero coordinated water molecules, based on Eu^{III} luminescence lifetime experiments and confirmed by the X-ray structure, combined with the presence of two Trp residues in the vicinity of EF2/3, suggested that it may be able to sensitize luminescence of not only Eu^{III} but also ions with shorter luminescence lifetimes, such as Sm^{III} and Dy^{III} (FIGS. 41-43). As shown in FIG. 41 and FIG. 42, Hans-LanM can sensitize Eu(III) and Tb(III) under time-resolved and steady-state conditions in which Mex-LanM performs much worse. FIG. 43 shows that the luminescence of Sm(III) and Dy(III) bound to Mex-LanM is extremely poor, whereas the signal is detectable for Hans-LanM. This could allow for sensitive detection of these metals in complex environments (analogous to Tb detection using Trp-LanM) or even within cells.

Characterization of Xanthobacter flavus LanM (LanM 012). The LanM sequence alignment combined with the structure led us to seek LanM proteins that might exhibit additional hydrogen bonding interactions between other EF hands, which might amplify protein's sensitivity among lanthanides for dimerization, alter dimerization strength, and/or allow for increased cooperativity because more EF hands would be interconnected. In the crystal structure, EF3 in one monomer appears to primarily linked with occupancy of EF3 in the other monomer (and EF4, but it is occupied by Na⁺). H25 (3 residues after EF1) and T40 (6^{th} residue of EF2) are interacting via a hydrophobic interaction between the His imidazole ring and the methyl group of the Thr. Conversion of this interaction into a hydrogen bond may be more stabilizing. In the X. flavus sequence, H25 is an Asp and T40 is a Lys, suggesting that they may hydrogen bond. The T29 equivalent is a Lys, which could also be involved in hydrogen bonding. The peptide sequence of LanM_012 for cytosolic expression (signal peptide removed, the equivalent residues to H25 and T40 in Hans-LanM, are underlined) is:

>LanM_012
(SEQ ID NO: 7)
MLTGKEFLRKYNKDKDSTVEIVEAIDLGTKVFKAINPDKDKTLEA
AETKGRLSDEDWAQENKDGDKTLELDEWLIIVRKRENDADANKDG
KLTEAELDAPAGQQLILLIAK*.

Gene sequence of LanM_012, inserted into pET29a as for Hans-LanM.

(SEQ ID NO: 50)
ATGCTCACAGGTAAAGAGTTTCTCCGGAAATATAATAAAGATAAGG
ACAGTACCGTCGAGATCGTCGAAGCGATCGATTTGGGTACCAAAG
TTTTTAAGGCGATCAACCCAGATAAAGATAAGACCCTCGAGGCTG
CCGAGACAAAGGGTCGACTTAGTGACGAAGATTGGGCACAGTTCA
ACAAGGATGGGGATAAAACATTAGAACTGGATGAATGGCTGATTA
TTGTTAGAAAAAGATTTAACGATGCTGATGCAAATAAGGATGGTA
AACTGACGGAAGCGGAATTGGATGCCCCAGCCGGACAGCAGCTGA
TCCTGCTGATCGCGAAGTGA

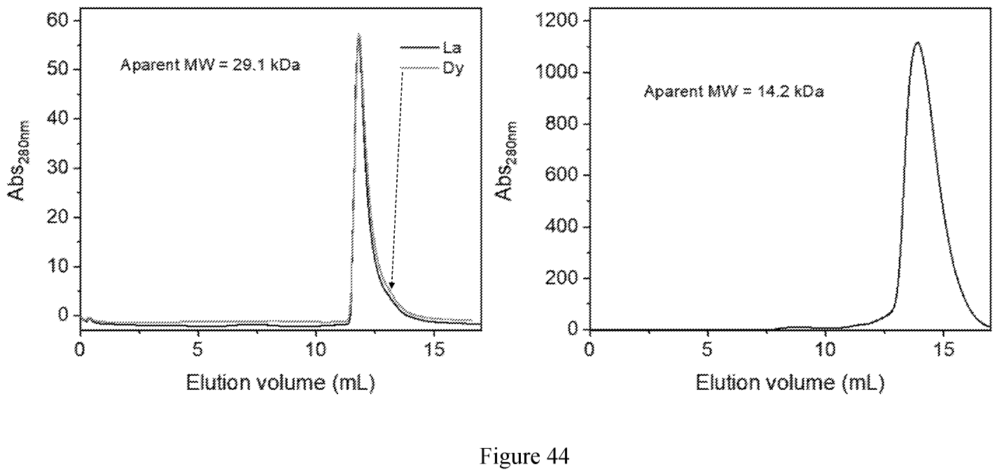

LanM_012 was expressed and purified as for Mex-LanM, yielding ˜10 mg/L. Adding an Ala as the first residue after the N-terminal Met may improve expression because of the N-end rule. FIG. 44 shows that, while the apoprotein elutes from an SEC column at a retention volume consistent with a monomer, while the La and Dy complexes elute as apparent dimers. This protein's propensity for dimerization shows that the presence of an R100 equivalent residue is very likely predictive of the ability of a LanM to dimerize, although other interactions not clear at the moment are responsible for overall strength of dimerization and difference in dimerization propensity for apo- vs. REE-bound proteins.

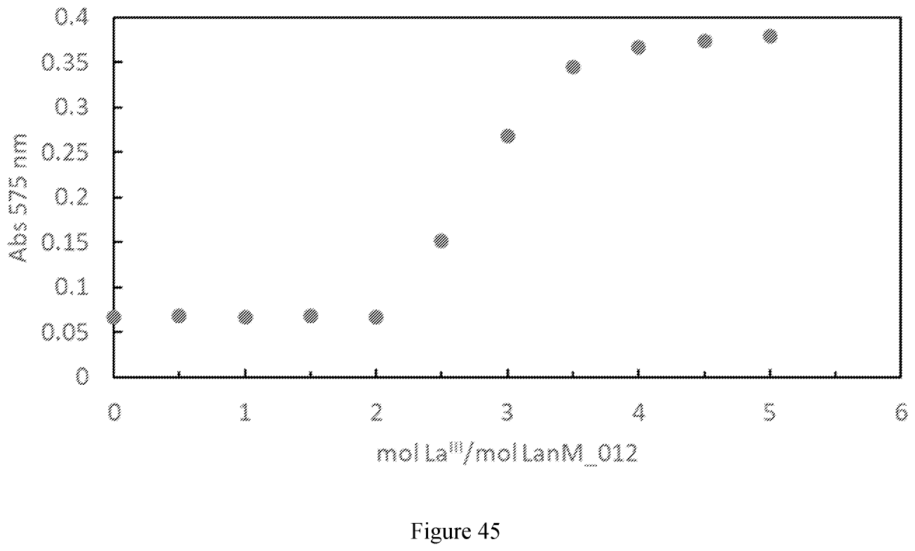

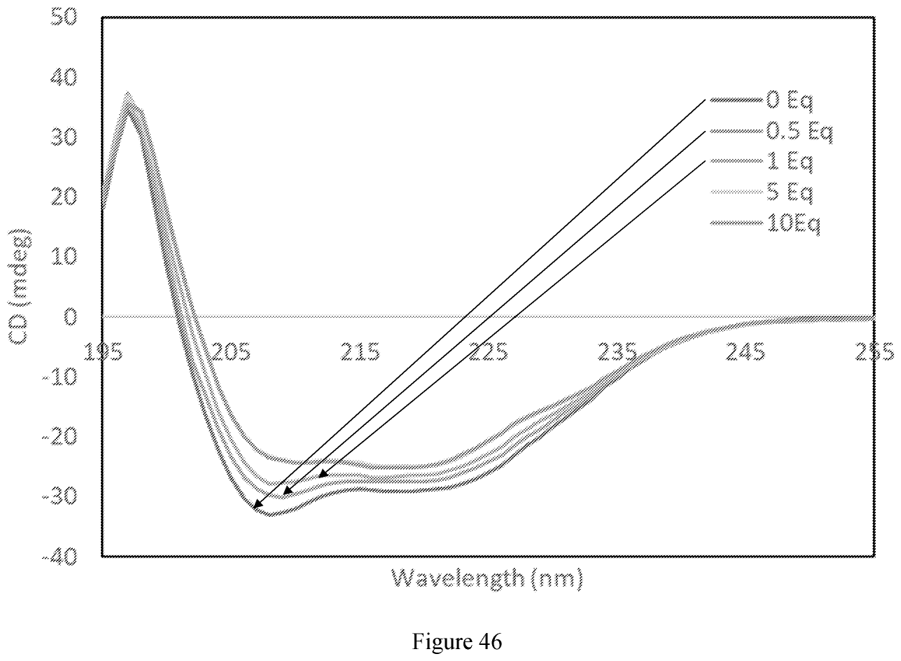

Like Hans-LanM, the third binding equivalent in LanM_012 is relatively weak, and is not able to outcompete xylenol orange, which has roughly micromolar affinity for REs (FIG. 45). However, we found that, at room temperature, apo-LanM_012 had helical character comparable to the LRE-bound states of Hans- and Mex-LanMs (FIG. 46).

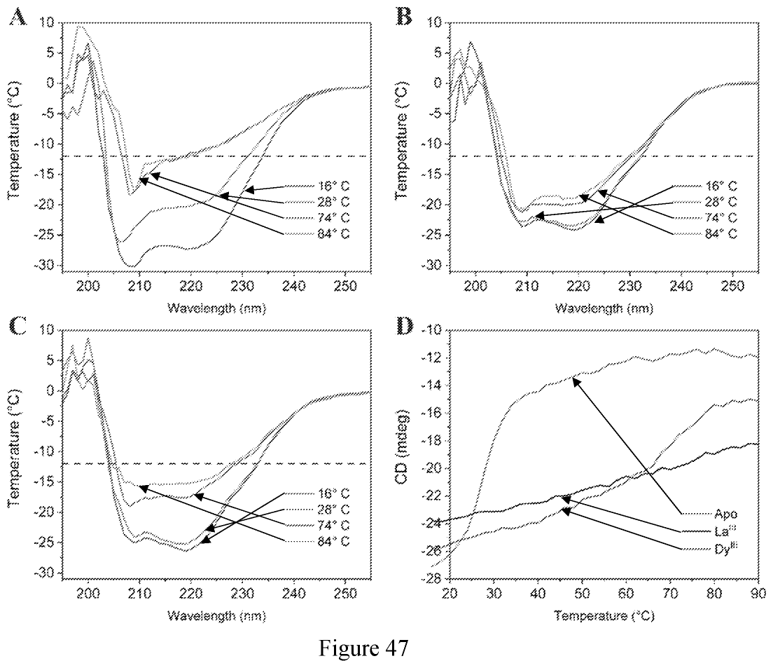

Although at room temperature the apoprotein has significant secondary structure, a temperature-dependence CD experiment shows that the stability of the helices is strongly dependent on temperature, as well as on the presence of metal, with the denaturation temperature increasing in the order apo<Dy<La, indicating increased stability of the LREE complex (FIG. 47). This suggests that high temperature could perhaps be used to selectively desorb HREEs from the protein.

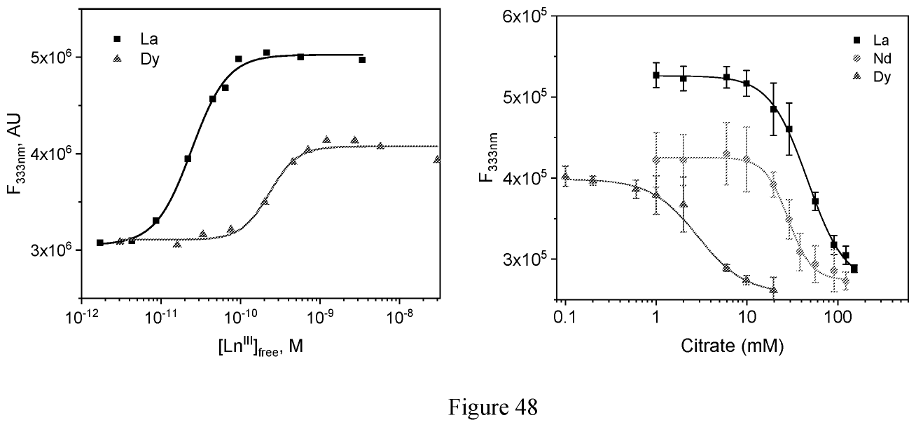

Titrations of the protein with buffered metal solutions and competitive titrations with citrate (FIG. 48, left and right) indicate that LanM_012 binds REEs more tightly than does Hans-LanM. Interestingly, as shown in the fitting parameters associated with these data (Tables 22 and 23), binding of both La(III) and Dy(III) are cooperative (n 2), which may result from the full dimerization in the presence of both ions (FIG. 44; compared to Hans-LanM, which is predominantly monomeric under these experimental conditions). However, it is noteworthy that the ΔF values for Nd and Dy are the same, and different from La, suggesting a difference in conformation between the La- and Nd-bound proteins, which might be possible to leverage for separation of LREEs (Table 23).

Table 22. Fitting parameters for FIG. 48 (left)

| Metal ion | K_{d, app} (pM) | n | Δ[F] |
|---|---|---|---|
| La | 24(2) | 1.9(2) | 2 · 10⁶ |
| Dy | 230(3) | 2.4(7) | 9 · 10⁵ |

Table 23. Fitting parameters for FIG. 48 (right)

| Metal ion | Citrate_{1/2} (mM) | n | Δ[F] |
|---|---|---|---|
| La | 47(3) | 2.1(2) | 2.6 · 10⁵ |
| Nd | 29(3) | 3.3(5) | 1.5 · 10⁵ |
| Dy | 2.9(5) | 1.7(2) | 1.4 · 10⁵ |

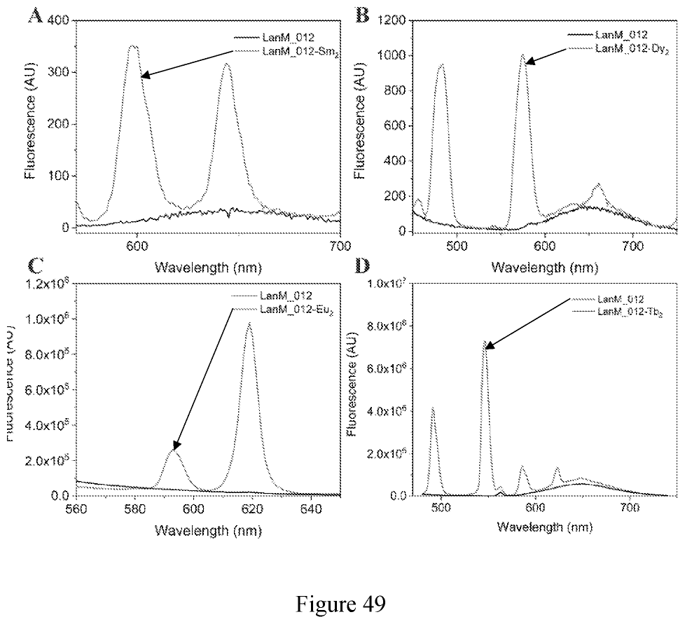

Finally, the similarity between the EF hand sequences of LanM_012 and Hans-LanM suggested that LanM_012 might also not have coordinated solvent, which would result in stronger luminescence of REE complexes, but also with a higher affinity that would be desirable for sensing applications. This expectation is validated by the results shown in FIG. 49. In turn, these data predict that the presence of a Glu at the 9^{th} position of the EF hands (perhaps in combination with an Asn at the 1^{st} position) can result in REE complexes without coordinated solvent.

Sequences. (additional sequences are given in Table 15)

>Hans-LanM (full protein sequence, signal peptide underlined)
(SEQ ID NO: 1)
MKLSLKAGAA ITAFVFAASP VLAASGADAL KALNKDNDDS LEIAEVIHAG
ATTFTAINPD GDTTLESGET KGRLTEKDWA RANKDGDQTL EMDEWLKILR
TRFKRADANK DGKLTAAELD SKAGQGVLVM IMK
>Hans-LanM (with signal peptide removed, as
expressed in this study)
(SEQ ID NO: 2)
MASGADAL KALNKDNDDS LEIAEVIHAG ATTFTAINPD GDTTLESGET
KGRLTEKDWA RANKDGDQTL EMDEWLKILR TRFKRADANK DGKLTAAELD
SKAGQGVLVM IMK
>Hans-LanM(R100K) (with signal peptide removed,
as expressed in this study, substitution
underlined)
(SEQ ID NO: 4)
MASGADAL KALNKDNDDS LEIAEVIHAG ATTFTAINPD GDTTLESGET
KGRLTEKDWA RANKDGDQTL EMDEWLKILK TRFKRADANK DGKLTAAELD
SKAGQGVLVM IMK
>Hans-LanM-Cys (for immobilization)
(SEQ ID NO: 5)
MASGADAL KALNKDNDDS LEIAEVIHAG ATTFTAINPD GDTTLESGET
KGRLTEKDWA RANKDGDQTL EMDEWLKILR TRFKRADANK DGKLTAAELD
SKAGQGVLVM IMKGSGC
>Hans-LanM(R100K)-Cys (for immobilization)
(SEQ ID NO: 6)
MASGADAL KALNKDNDDS LEIAEVIHAG ATTFTAINPD GDTTLESGET
KGRLTEKDWA RANKDGDQTL EMDEWLKILK TRFKRADANK DGKLTAAELD
SKAGQGVLVM IMKGSGC

FIG. 27 contains a non-exhaustive list of sequences likely to dimerize (containing an R100 equivalent residue, position 87 in the figure, in addition to the residues in positions 72, 78, and 80 as shown in that figure—corresponding to D85, E91, D93 in the main text figures). These sequences are compared to sequences, like M. extorquens, unlikely to dimerize as well as a few other specific sequences discussed in the legend. These include the following:

>LanM_011 (Hyphomicrobium, with signal
peptide removed)
(SEQ ID NO: 14)
MGHHNCKAEMAYINPDHDGTIDWREARRAAVRLFHKLDPDHDG
TLDMKEVRGRVGILSFARENPDRDGKLDKHEWLALVKHRFHRA
NPDKDGTIDCRELHSLAGRKLLRVLM
>LanM_012 (Xanthobacter flavus, with signal
peptide removed)
(SEQ ID NO: 7)
MLTGKEFLRKYNKDKDSTVEIVEAIDLGTKVFKAINPDKDKTL
EAAETKGRLSDEDWAQFNKDGDKTLELDEWLIIVRKRFNDADA
NKDGKLTEAELDAPAGOQLILLIAK
>Unclassified Hyphomicrobium (signal peptide
removed; appears to only have 3
functional EF hands)
(SEQ ID NO: 15)
MGHRSAKAHPSCPALNAIDPDGDGAMTLGEAKRAAIKTEMKLN
KDGDITLELDELGGRMSAAAFAQADLIKGRGISLGEYLIEVRR
RFKWANPDKDHTIECDELHSKYGRLLARLLK

A non-limiting set of variants is the following:
Constructs with two Hans-LanM monomers connected by different amino acid linkers (Hans-linker constructs):
HansR100K-L1
HansR100K-L2
HansR100K-L3
HansR100K-L4
HansR100K-L5
Constructs with Hans-LanM interfacial residues mutagenized to alter dimerization affinity (all in the Hans-LanM background):
I143A, I43V
A44N, A44S, or A44T
I47A, I47V
M92L, M92A, or M92D
D93N, D93A, D93E
Combinations of successful Hans-linker constructs with the Hans-LanM interfacial residue variants
Mex-LanM variants to potentially alter metal binding stoichiometry/affinity:
A32D/A117K
A32D/A117R
I42L/N108D/I115L
G51A
A98G
A99G
V100G
A102G
N108D
A124G
A127G
Combinations of the above

Features important for RE-dependent protein dimerization. It is anticipated based on results that the following properties are sufficient for dimerization by the mechanism described: 1) A LanM-like sequence. First, most have four EF-hand motifs, though proteins with 2 or 3 EF-hands that possess the second and third properties listed below are possible (however, such proteins may or may not dimerize via a distinct mechanism/interface). Second, except in the cases of the 3 EF-hand proteins (in which one of the EF-hands has been corrupted such that few carboxylate residues are present) the EF-hands are separated by roughly 12-13 residues (12-13 residues between the terminal amino acid, usually Glu, of one EF hand, and the first amino acid, usually Asp or Asn, of the next. This spacing is, to the best of our knowledge, unique among EF-hand proteins and may itself be diagnostic of a LanM. Third, at least one EF hand contains proline at the second position (in Mex-LanM, all four EF hands feature P2 residues). Using the first two criteria and a sequence length of <200 residues, we identified 696 putative LanMs. 2) The following pattern of residues, at the sequence positions aligning with Hans-LanM D85, E91, D93, and R100, where the D85, E91, and D93 equivalents are at the 3^{rd}, 9^{th}, and 11^{th} positions of EF-hand 3, and D93 and R100K being the residues that appear to be unique in enabling dimerization that is sensitive to the identity of the RE bound to the protein. Structures show that other interactions are also involved in stabilizing the dimer, but the above residues are conserved in many LanM proteins and appear to be key for RE-sensitive dimerization.

Note that, in principle, the full protein sequence may not be necessary for RE-dependent dimerization, e.g., not all of the residues in or in the vicinity of EF1 and EF4 in primary sequence may be required. In other words, shorter (more atom-economical) LanM-like proteins also capable of RE-sensitive dimerization can be envisioned.

Additionally, the trend in selectivity between REEs, the steepness of the inter-REE selectivity, and the overall strength of dimerization can be modulated (decreased or increased) by mutagenesis of interfacial polar and non-polar residues as described herein.

Finally, other conceptually related mechanisms of dimerization may be possible in other lanmodulins (e.g., the use of different dimerization interfaces by exploiting a specific interaction between EF2 ligands), and indeed in other lanthanide-binding proteins.

### Example 3

This Example describes use of the peptides/proteins of the present disclosure.

LanM_013 was of interest because EF2 is similar to a Hans-LanM EF hand (Ni residue and E9 residue, with a Gly at position 4) whereas EF3 is similar to a Mex-LanM EF hand, and all EF hands contained Pro residues. It was envisioned that the structure of EF2 could be very sensitive to RE identity (as Hans-LanM EF hands are), but the protein was not expected to dimerize, so it was possible that it might show increased selectivity within the lanthanide series within a single LanM unit.
>LanM_013 (expressed construct; signal peptide removed and N-terminal Met added EF hands underlined):

(SEQ ID NO: 8)
MGKAADAIQALDPDKDGTIDLNEAKAGAKAVFEKINPDGDGTLE
VKELKGRLTKKELDAADPDNDGTLDMQEYEAVVTKQFELANPDN
DGTVDEKELKTKEGKKLLKLIY

>LanM_013 (full-length sequence from Methyloligella sp. GL2, with signal peptide, underlined):

(SEQ ID NO: 9)
MAAILTIAGAVTVAAGGAAFAGKAADAIQALDPDKDGTIDLNEA
KAGAKAVFEKINPDGDGTLEVKELKGRLTKKELDAADPDNDGTL
DMQEYEAVVTKQFELANPDNDGTVDEKELKTKEGKKLLKLIY

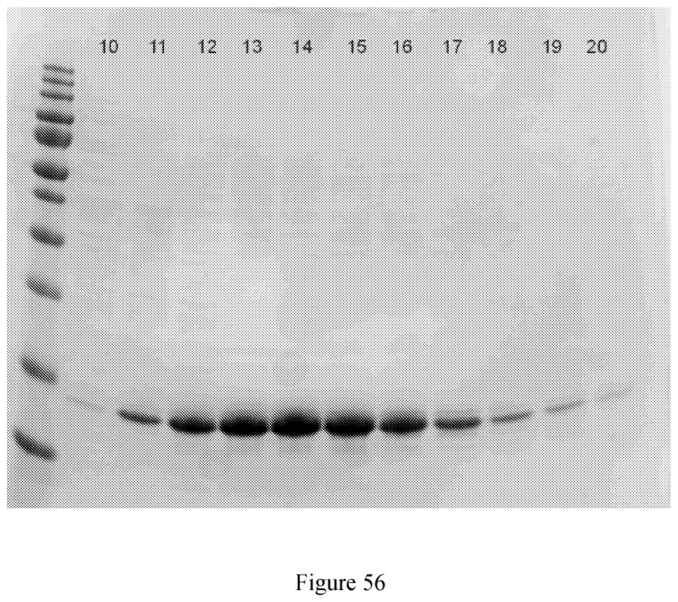

The procedures used for expression in E. coli and purification of LanM_013 were identical to those used for Mex-LanM, except that no Ca(II) was included in the gel filtration step. FIG. 56 shows the high purity of this protein after this purification procedure. The elution volume of the protein from the S75 column was consistent with a monomer. The purification yielded 80 mg LanM_013 from 2 L culture (40 mg/L). The protein possesses two Tyr residues; an extinction coefficient at 280 nm of 2980 M⁻¹ cm⁻¹ was used.

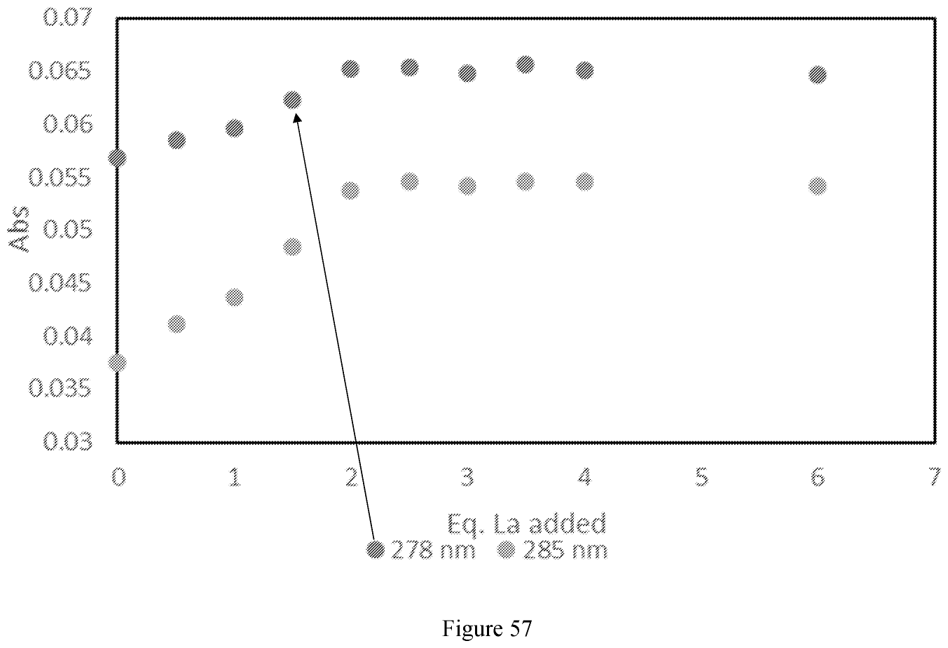

FIGS. 57-59 address the binding stoichiometry of LanM_013. In FIG. 57, peak absorbances are followed at both 278 nm and 285 nm are shown for clarity. Both of these peaks reach a maximum after addition of 2.0 equivalents of La(III). Note that this assay specifically reports on the environment of the tyrosine residues in the protein, which are in the vicinity of EF hands 2 and 3. The total number of equivalents of metal that bind to the protein may be higher.

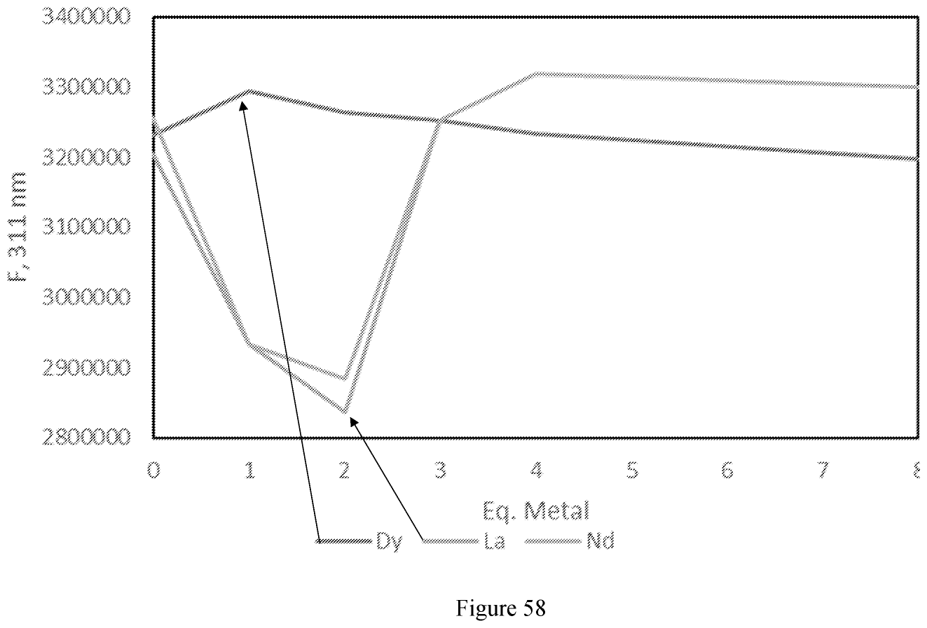

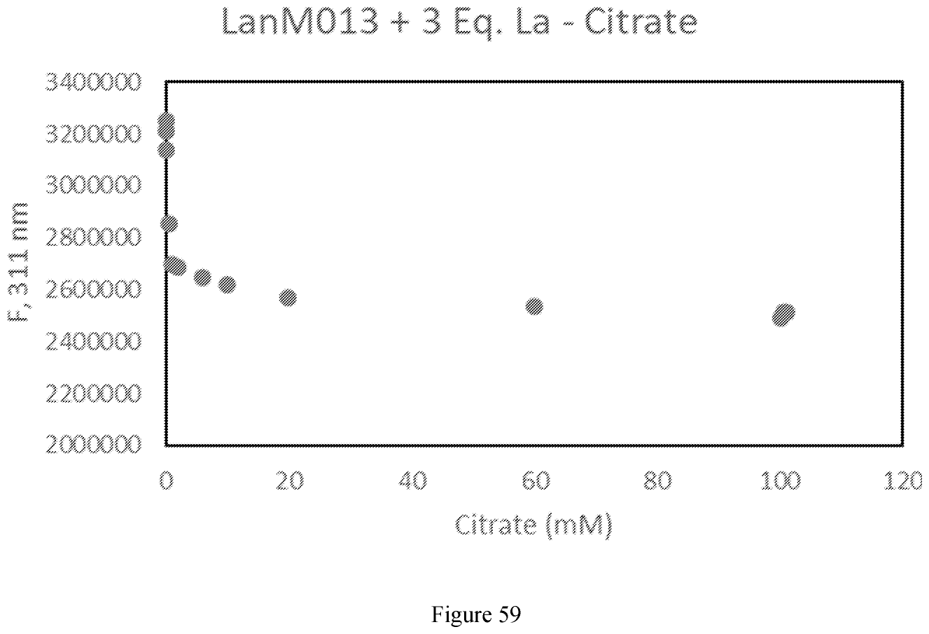

FIG. 58 shows fluorescence emission of the Tyr residues in LanM_013 in titrations with La(III), Nd(III), and Dy(III). An initial drop in fluorescence for the first two added equivalents of La(III) and Nd(III) is followed by an increase for the next ˜1.0 equivalent, similar to Mex-LanM, showing that under these conditions, the protein can bind at least 3 equiv. La(III) and Nd(III). Interestingly, this was not observed for dysprosium, indicating that there is a structural difference in the vicinity of one or both of the protein's Tyr residues when LanM_013 is bound to certain lanthanides. FIG. 59 uses a competitive titration of the La(III)-bound LanM_013 (3 equiv. La(III)) with citrate, again monitored by the intrinsic tyrosine fluorescence, to estimate how tightly La(III) is bound to the protein at its 3 highest-affinity sites. The initial loss of fluorescence is attributed to a weaker binding third equivalent of metal (see FIG. 57). Even 100 mM citrate was not able to recover tyrosine fluorescence to that of the apoprotein, indicating that the remaining two equivalents of metal, putatively bound to EF2 and EF3, are tightly bound, perhaps more tightly than to Mex-LanM. For comparison, the tightest two equivalents of La(III) are desorbed ˜50% at ˜20 mM citrate (see FIG. 4a of the Hans-LanM work). Slower equilibration kinetics might also play a role in the LanM_013 system.

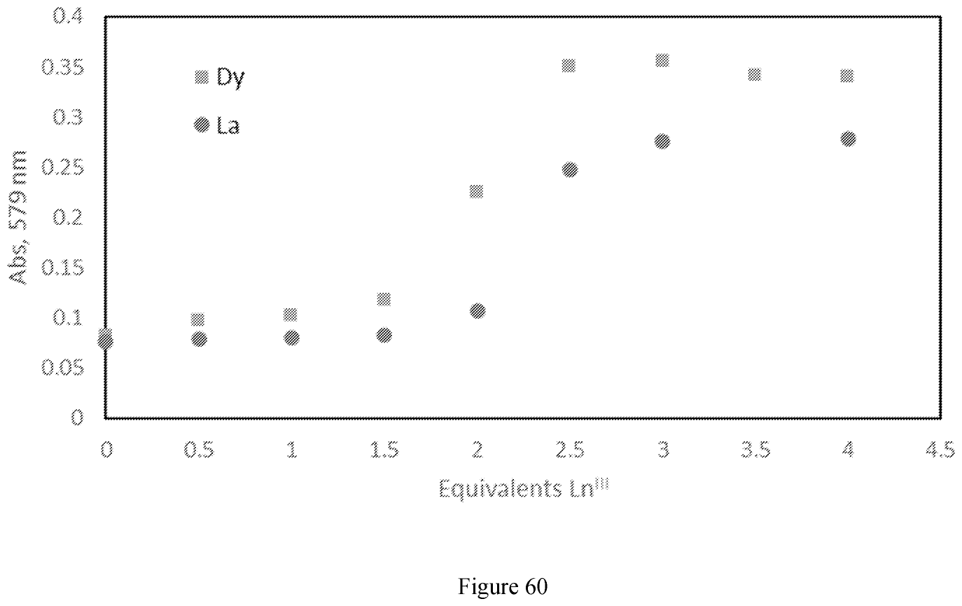

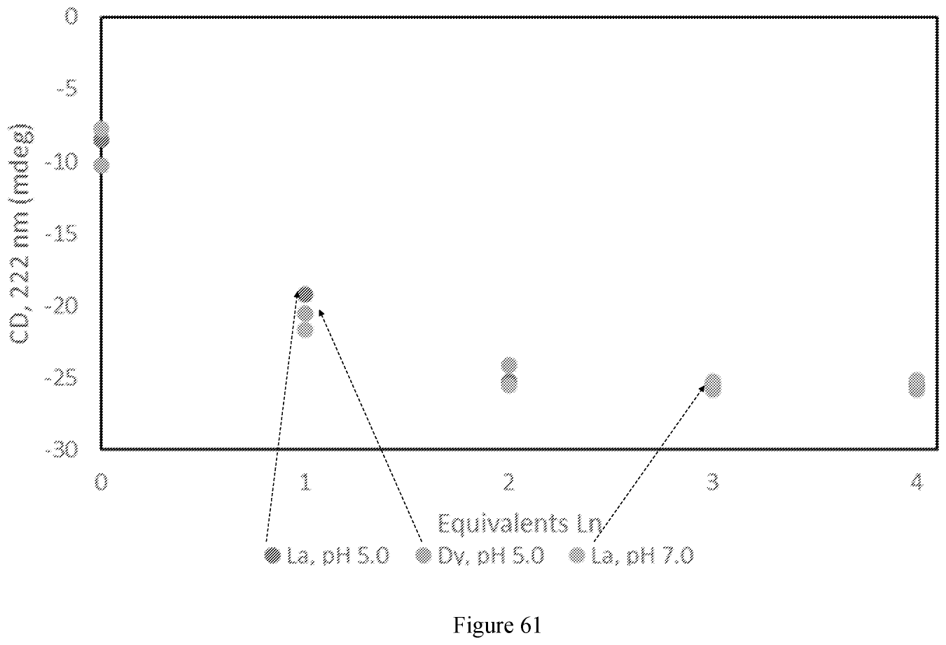

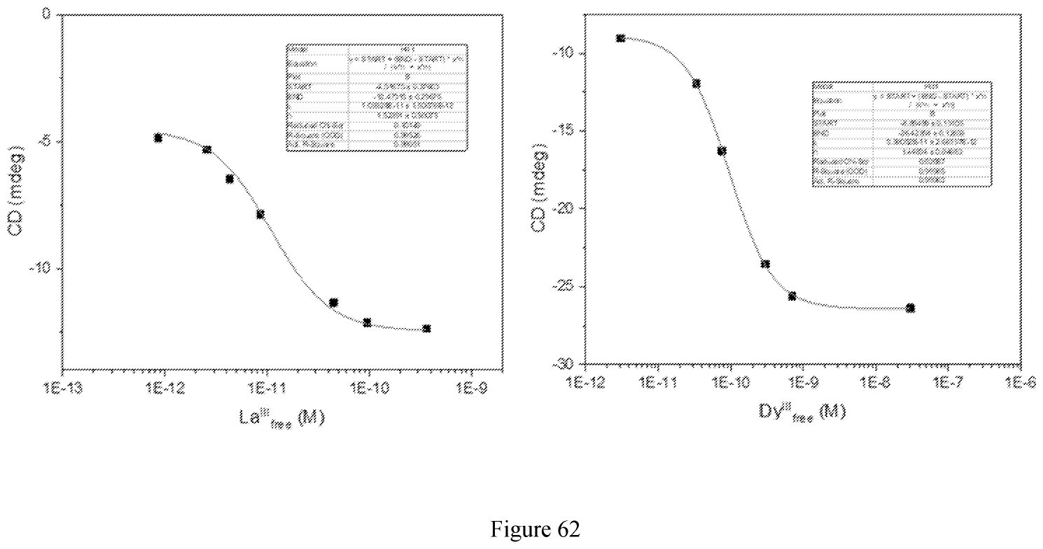

FIGS. 60 and 61 address the stoichiometry and affinity of LanM_013. In FIG. 60, xylenol orange was used as a colorimetric competitor for La(III) and Dy(III) binding. Results show a small increase in signal throughout the Dy(III) titration, with significant increase occurring between 1 and 2 equivalents of added metal, indicating relatively weaker binding to Dy(III) compared to La(III). La(III) clearly shows ˜2.0 equivalents of binding, which corresponds well with the other experiments above. The third equivalent of La(III) binding evident in the fluorescence experiment is presumably weak; it would not be observed in the xylenol orange assay if it is weaker than ˜1-10 μM. In FIG. 61, CD spectroscopy is used to assess the extent of LanM_013's conformational changes due to lanthanide binding. A clear titration endpoint was seen for both La(III) conditions (pH 5 and pH 7), whereas a broader response was observed for Dy(III). This suggests that the affinity of at least one site in the Dy(III)-LanM_013 complex is likely weaker than the La(III)-LanM_013 complex. In FIG. 62, CD spectroscopy is used to preliminarily estimate that the apparent K_{d} values for LanM_013's response to certain lanthanides are 11 μM (La) and 96 μM (Dy). Both Hill coefficients are ˜1.5.

These experiments allowed conclusions to be drawn, even in the absence of more detailed affinity determinations. LanM_013 has several intriguing properties that might make it well suited for recovery of only LREs or LRE/HRE separations. Metal binding to the tightest two binding sites (which we posit are EF2/3 based on the other characterized LanMs) appears to lead to a different enough structure in the vicinity of the Tyr immediately following (Y73) that that residue's fluorescence emission quenches in the presence of La(III) and Nd(III) but not Dy(III). The other biophysical data also point to a larger affinity difference of at least one of those sites between heavy and light REs, though we need to assess where in the lanthanide series the affinity “breakpoint” occurs. In this regard, this protein may have achieved an effect like in Hans-LanM but within a single polypeptide. The two tightest sites also appear to be tighter for LRE binding than Mex-LanM.

The insights from the crystallographic characterization of the Mex-LanM and Hans-LanM systems enabled predictions about the origins of the key properties of this new protein, and therefore potential extensions to proteins with similar sequence characteristics that have not yet been biochemically characterized. First, regarding the apparent structural differences between Dy- and La/Nd-bound LanM_013: the E9 residue in EF2 (E44) may undergo a change from bidentate (with LREs) to monodentate (with HREs) coordination, which may be transduced in some way to EF3 such that pairing of EF2 and EF3 can only occur when a LRE is in each EF hand. The presence of a second Gly in EF2 (G39) may also play a role in making HREs bound to EF2 produce a conformation incompatible with interaction with EF3. The Asn as first residue in EF2 may also contribute. Second, the apparent higher affinity of the protein for La(III) than Mex-LanM, based on the citrate data, a potential source of higher affinity could be additional hydrogen bonding interactions specific to the LRE-bound form of the protein. Based on comparison to the Mex-LanM structure, the residue E74 (Glu just after EF3) may be positioned well to be engaged in a hydrogen bond with K29 and/or K25. Such interactions may also contribute to a difference in affinity for LRE vs. HRE complexes with the protein.

### Example 4

This Example describes use of the peptides/proteins of the present disclosure.

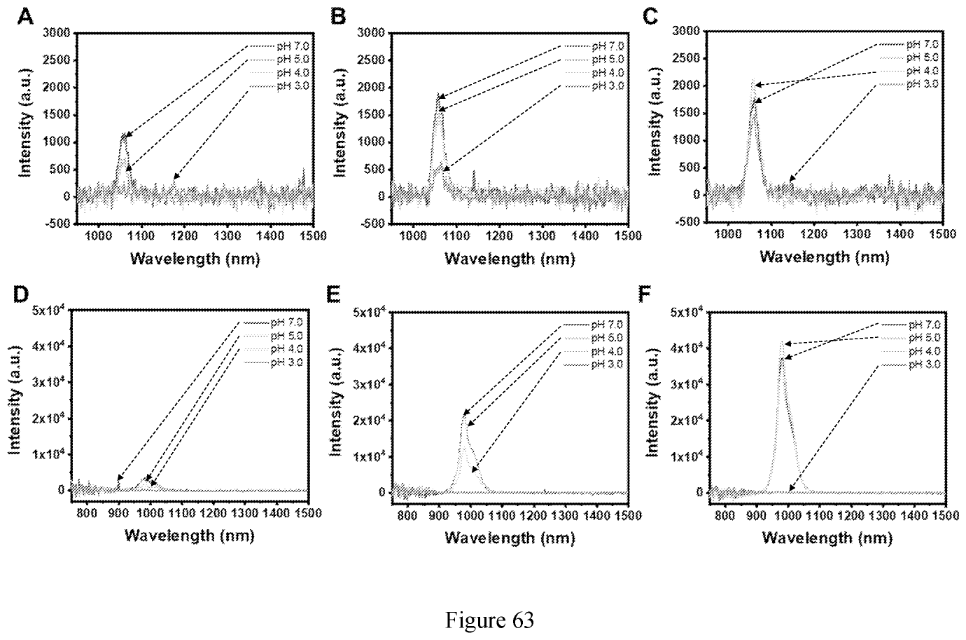

Nd and Yb sensing using LanM_012. The ability of Mex-LanM(T90W), Hans-LanM(R100K), and LanM_012 to sensitize the near-infrared (NIR) luminescence of Nd(III) and Yb(III) was tested. The data are presented in FIG. 63. The data show that all three sensors can sensitize Nd(III) and Yb(III) above pH 4.0. Only LanM_012 exhibits considerable intensity for Nd(III) at pH 3.0, and none of the sensors were able to sensitize Yb(III) at pH 3.0, potentially due to the low binding affinity of LanMs for later REEs under low pH conditions. Overall, LanM_012 is the best performer overall for both ions, but especially for Yb(III), which exhibits very high luminescence intensity. Therefore, from all the work contained herein, LanM_012 is an especially promising protein-based sensor for several individual REEs—Tb, Eu, Dy, Sm, Nd, and Yb—and may be useful for in vitro REE studies and in-cell REE uptake studies.

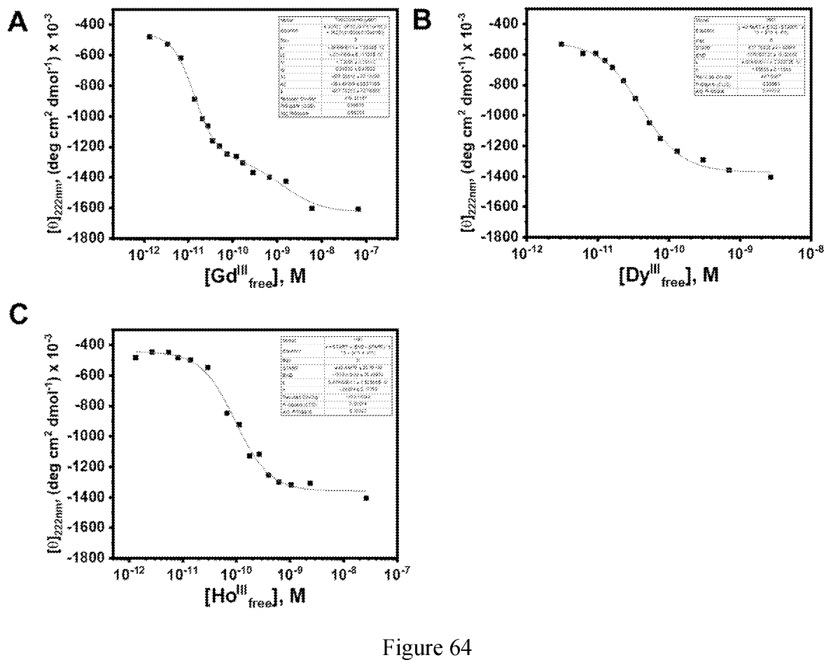

Determination of dissociation constants (K_{d}s) of LanM_013. The apparent dissociation constants of LanM_013 for three middle-late REEs with similar ionic radius were determined at pH 5.0 (Table 24 for summary, FIG. 64 for plots). LanM_013 has a preference for lighter lanthanides such as Gd(III) over heavier ones like Dy(III) and Ho(III). In these titrations with single REEs, there is rather steep affinity trend over a narrow range of atomic numbers: ˜7-fold difference between Gd and Ho (only Tb and Dy are between them). The apparent K_{d} of LanM_013 for Gd is ˜3-fold tighter than for Dy, and Dy is ˜2.5-fold tighter than for Ho. Additionally, there appears to be a trend that binding is more cooperative for light lanthanides than for heavy lanthanides. LanM_013 displays a bi-phasic response to Gd(III), with a cooperative major phase and non-cooperative minor phase (FIG. 64A). A similar curve could potentially be observed for Dy(III) and Ho(III) if measured at higher metal concentrations—because this potential second phase was not considered in the Dy and Ho fitting, the apparent Kds may vary slightly from the numbers reported here. It is not known at present whether the weaker phase is EF1, as it seems to be for other LanMs that have been studied. It should be noted that these Kas are slightly tighter than those reported previously, likely because higher protein concentrations were used previously (20 μM vs. 10 μM), which slightly impacted the free metal concentration in the titrations such that the calculated free metal concentration value was slightly overestimated.

Table 24. Summary of fitted parameters for CD titrations of LanM_013 with Gd^{III}, Dy^{III}, and Ho^{III}.

| Metal ion | K_{d, app} (pM) | n |
|---|---|---|
| Gd | K_{d1}: 13.7 | n₁ 1.7 |
| | K_{d2}: 1230 | n₂ 1.0 |
| Dy | 40.0 | 1.5 |
| Ho | 95.4 | 1.4 |

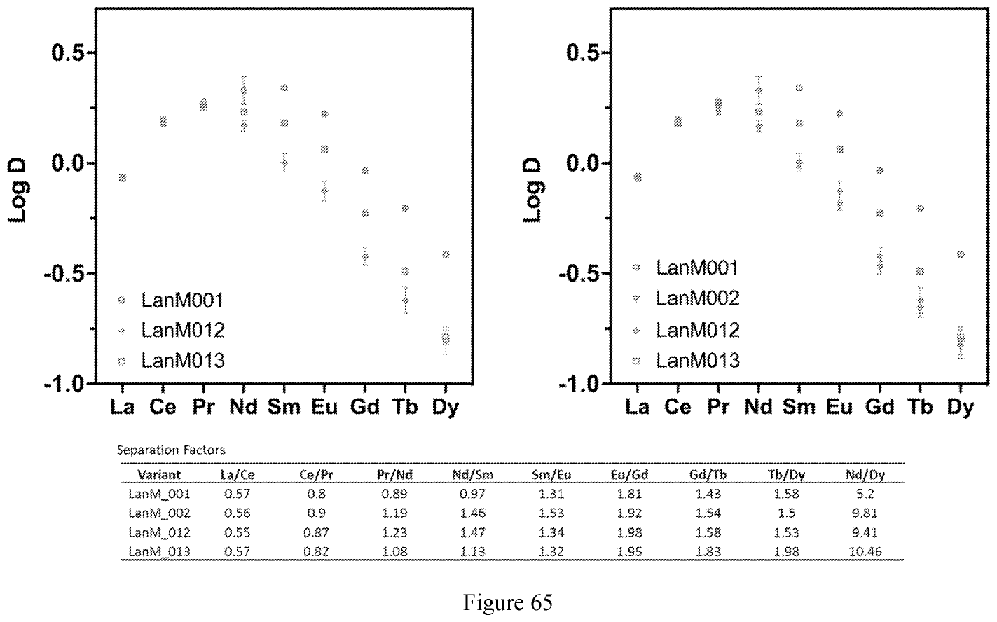

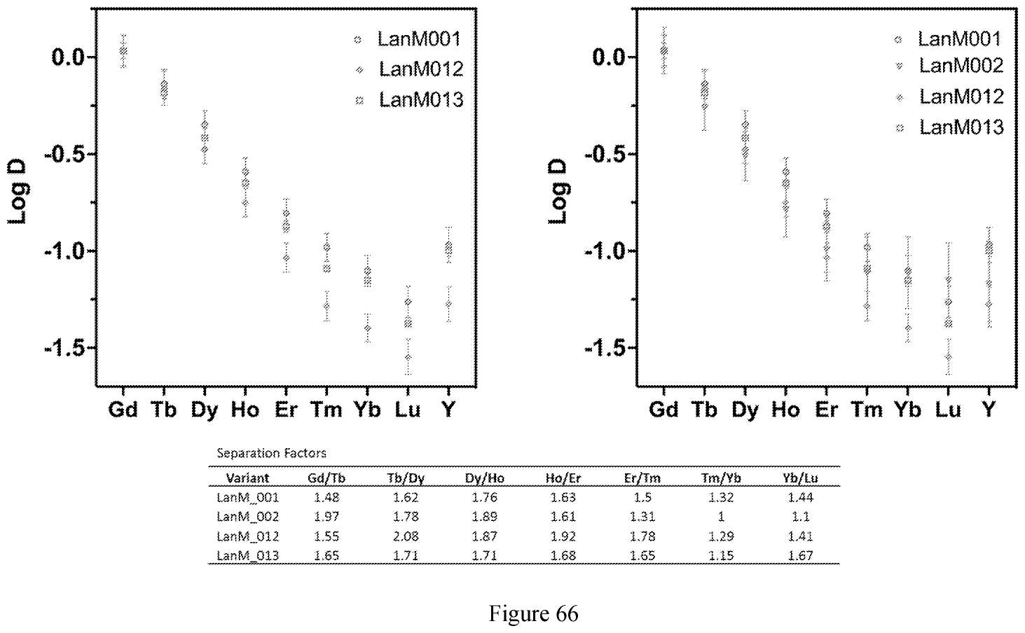

On-column intra-REE selectivity of LanM_013 compared to other orthologs. To quantitatively determine the intra-REE selectivity of different lanmodulins, equilibrium binding was performed with immobilized protein using separate light (La-Dy—FIG. 65) or heavy (Gd-Lu with Y—FIG. 66) REE mixtures, where each metal ion was added at an equimolar concentration (pH 5). Each solution was circulated over the LanM column for two hours. Distribution values (D) were determined by quantifying the distribution of REEs between the solid (i.e., LanM bound) and the aqueous phases. The D values were used to determine separation factors by taking the ratio of distribution values between two REEs of interest. For LanM_001, the highest selectivity was observed for the L- and MREEs Ce, Pr, Nd, Sm, and Eu, which formed a selectivity plateau exhibiting minimal differences between D values. LanM_002 and LanM_012 exhibited a narrower selectivity plateau centered around Pr and had substantially enhanced selectivity between LREEs and HREEs. LanM_013 exhibited an intermediate selectivity trend relative to LanM_001 and LanM_002/LanM_012, which included preferential binding to Ce-Sm. Interestingly, LanM_013 exhibited the steepest selectivity among the variants for Eu through Dy, yielding separation factors close to 2 for Eu/Gd, Gd/Tb, and Tb/Dy. Compared to LanM_001, these variants are poised to enable more effective separations of adjacent REEs from Pr through Dy. It is worth noting that, in the Gd-Lu,Y results for LanM_013 (FIG. 66), the selectivity trend looks much closer to LanM_001 than would be expected from the La-Dy data (FIG. 65). It is possible that the presence of LREEs enhances the selectivity among HREEs, perhaps through a mixed binding scheme.

Experimental details: Batch experiment to determine distribution values and separation factors. LanM immobilized microbeads were washed with DI-water. 5 mL of feed solution (total 3 mM REEs, equimolar at pH 5) was added to 1 mL microbeads and incubated for 2 h. The liquid at equilibrium was collected and REE concentrations were determined as [M] adsorption. Then 4 mL of 0.1 M HCl was used to desorb REE in which concentrations were measured as [M]desorption. REE distribution values (D) between the LanM phase and solution phase were calculated as:

$$D = \frac{{[ M ]}_{LanM}}{{[ M ]}_{Liquid}} \qquad ( 1 )$$

where [M]_{LanM} and [M]_{Liquid} are the metal ion concentrations in the LanM solid phase and unbound solution phase at equilibrium, respectively. To account for the free liquid that absorbed within the agarose microbead, the following correction was carried out [M]_{liquid}=[M]_{adsorption}; [M]_{LanM}=([M]_{desorption}*4−[M] adsorption*1)/4. Separation factor (SF) is defined as:

$$S F = \frac{{D}_{REE 1}}{{D}_{REE 2}} \qquad ( 2 )$$

where D_{REE1} and D_{REE2} are distribution values of REE1 and REE2, respectively.

Improving the binding capacity of Mex-LanM by stabilizing metal binding to EF1 and EF4. The target sites to enhance binding stoichiometry are EF1 and EF4. EF4 demonstrates very weak metal binding, whereas the affinity of EF1 is slightly lower than that of EF2 and EF3 at neutral pH, but it becomes much lower as pH decreases. It is hypothesized that the increased lability of EF1 is due in part to its pairing with EF4, which does not bind to a metal ion tightly and is consequently more pH-sensitive. Furthermore, the low affinity of EF4 might be related to preorganization (i.e., its adjacent helices are stabilized by metal binding to EF2 and EF3).

To achieve tighter REE binding to EF1 and EF4, site-directed mutagenesis was employed with three different strategies. First, the metal binding loops in EF1 and EF4 by was optimized replicating the amino acid sequences found in EF2 and EF3. Notably, there is a difference in EF4, where Asn occupies the first position (from here on, the subscript number denotes the position within an EF-hand) instead of the Asp₁ found in the other three EF-hands. The nonbinding sidechain NH₂ of Asn₁ is unable to form a hydrogen bond with the backbone NH of Gly₆, inducing a different conformation of the loop. Perhaps for this reason, the 8^{th} position of EF1 and EF4 are Ile, instead of Leu₈, as they are in EF2 and EF3. Hence, mutations changing Asn₁ to Asp in EF4 and altering Iles to Leu in EF1 and EF4 might optimize the binding geometry for lanthanides. (Variant group 1: N108D and I42L N108D I115L).

Additionally, the introduction of hydrogen bonds between EF1 and EF4 could enhance the stability of the complex in a metal-dependent fashion. In particular, the crystal structure of Nd(III)-bound Mex-LanM revealed a hydrogen bond linking the −3^{rd} (D56) position of EF2 with the 10^{th} (K93) position of EF3. On the other hand, Ala residues occupy the equivalent positions in EF1 and EF4. Therefore, mutating these Ala residues in a manner analogous to EF2 and EF3 could establish a stabilizing hydrogen bond (Variant group 2: A32D/A117K and A32D/A117R).

It is postulated that EF4 is pre-organized in a manner that results in less energy being released during metal-induced conformational changes. As such, it was hypothesized improved stoichiometry can be achieved by destabilizing the apo binding loops. This would activate EF4 for metal binding and, in turn, stabilize the metal-bound conformation of EF1. In wt-Mex-LanM, EF2 and EF3 occupancy leads to the majority of α-helical content of the protein. It is proposed that the α-helix (α2) between EF3 and EF4 is entirely or largely formed upon REE binding to EF2/3; therefore, inhibiting the complete formation of α2 during EF2 and EF3 metal chelation could leave some stabilization to occur upon EF4 occupancy. Thus, three Ala residues, which tend to stabilize helices, roughly in the middle of α2 have been selected for mutations to the more flexible Gly, which tends to destabilize helices (Variant group 3: A98G, A99G, and A102G).

The binding stoichiometry of the variants was examined using the xylenol orange (XO) competition assay and circular dichroism (CD) spectroscopy. The buffer used for the XO competition assay was 20 mM acetate, 20 mM MES, 100 mM KCl, pH 6.1. CD spectroscopy was employed to observe the conformational changes of variants upon Nd titration. The buffer for CD was 20 mM acetate, 100 mM KCl, pH 5.0.

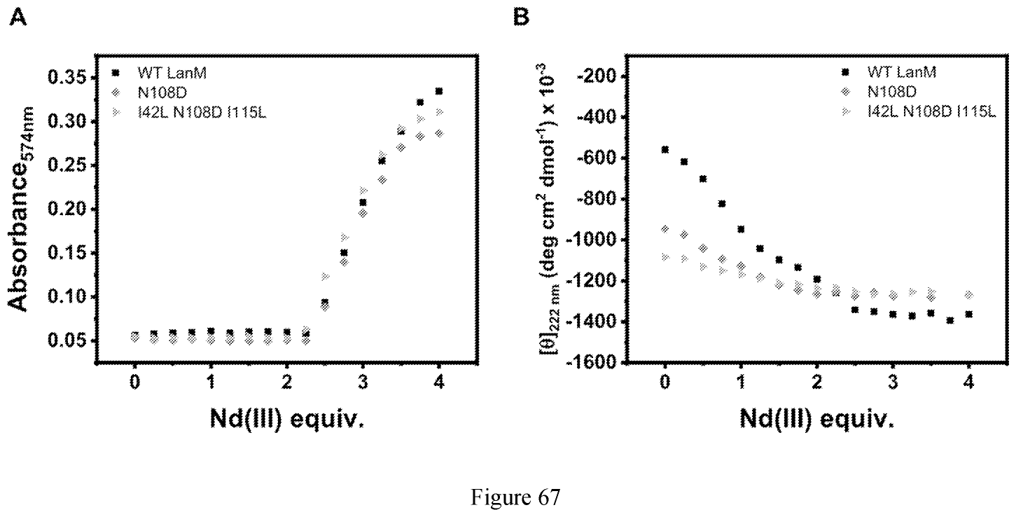

Variant group 1: None of the variants demonstrated improved binding stoichiometry. However, it was found that the apo N108D possesses significant α-helical content at pH 5.0—nearly half of the maximum helicity (FIG. 67B). When there is an Asp at this position in EF4, it might form a hydrogen bond with the backbone NH of the Gly at the 6^{th} position, leading to the generation of α-helices around EF4. Presumably, these generated α-helices could be located between EF3 and EF4 and after EF4 to the C-terminus. The mutations I42L and I115L seem to further stabilize the apo structure in conjunction with N108D, but the effect is small.

Variant group 2: The A32D/A117R and A32D/A117K variants displayed an improvement in binding stoichiometry by ˜0.5 equivalents at pH 6.1, as determined by the XO competition assay (FIG. 68A). This same effect was not observed from the CD titration, although it should be noted that the CD titration was conducted at pH 5.0, so this may reflect pH dependence of the hydrogen bond strength (FIG. 68B). The secondary structure of these variants is similar to that of the wt LanM. The additional hydrogen bond might contribute to stabilization the EF1 and EF4 complex, but it could potentially require additional mutation(s) (e.g., stabilization of metal-bound EF4) for full effect.

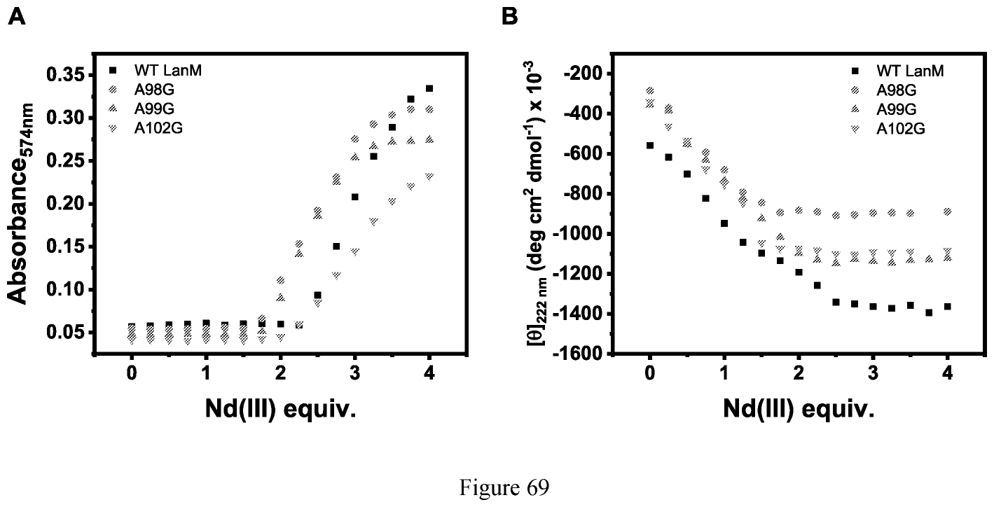

Variant group 3: None of the variants displayed improved binding stoichiometry. A98G and A99G seem to destabilize EF1 binding (and possibly EF2/3), whereas the effect of A102G on stoichiometry is minimal (FIG. 69A). However, all A to G variants exhibit both reduced α-helicity in their apo state and less helicity than wt in the metal-bound state (FIG. 69B). The reduced helicity of the apo state in these variants suggests that α2 is partially ordered in the wt apoprotein, or it is helping to stabilize another portion of the protein, such as the α-helix after EF4 to the C-terminus.

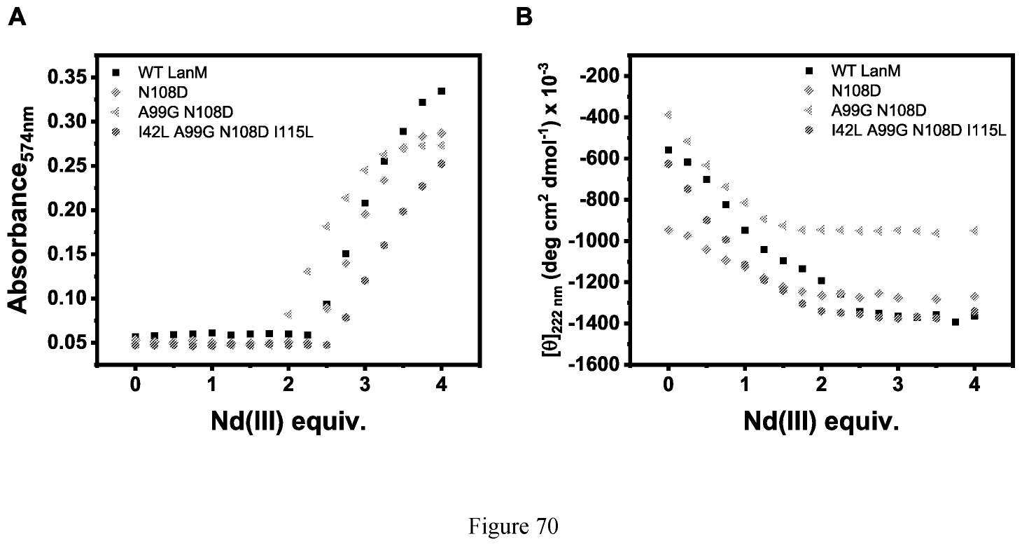

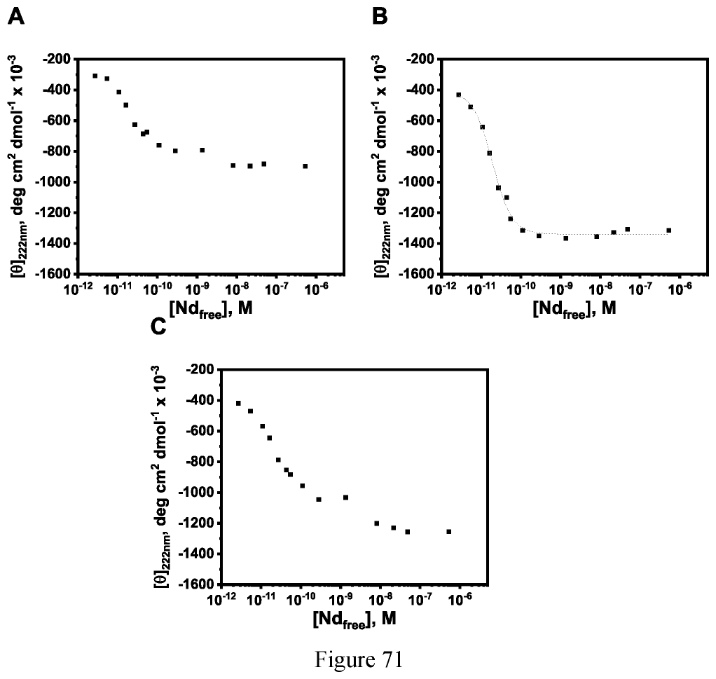

“Combo” variant group: A variant group called “combo,” which combines mutations from variant groups 1 and 3 (FIG. 70), was prepared. Among the options, A99G was chosen because the CD-titration curve seemed to display an improved second binding event (FIG. 71B). Interestingly, the combination of A99G and N108D mutations reduced the α-helicity of the apo N108D back to near-wt levels. This observation suggests that giving the residue 108 the capacity to hydrogen bond with the G113 backbone NH directly or indirectly leads to communication with and stabilization of the α2 helix, which also would communicate with the C-terminal helix. On the other hand, since the A99G-containing variant exhibited neither a complete conformational change nor improved binding stoichiometry, it may not be an appropriate position for mutation. The variant I42L A99G N108D I115L exhibited a minor improvement in Nd binding stoichiometry in the XO competition assay, but the CD results indicated decreased binding stoichiometry at pH 5.0.

Therefore, to stabilize metal binding to EF1 and EF4, the results suggested the following. First, A32D with a basic residue (most likely R) at residue 117 slightly stabilizes EF1, but not enough to confer stabilization at pH 5 and below. Upon further stabilization of EF4 metal binding, it is believed that the A32D/A117R mutation can be combined with the EF4-stabilizing mutations to fully stabilize EF1 as well. Second, it is believed that N108D is essential for forming an optimized EF4 binding loop, but it must be combined with mutations that destabilize helices in the apoprotein. Introducing Gly residues strategically to effect this destabilization appears to be important, but A99G may not be the right choice. Retaining the N108D mutation, an A to G mutation will be introduced in the region after EF4. The two Ala residues after Pro123 have been selected: variants N108D A124G and N108D A127G will be expressed, purified, and subsequently characterized in a similar manner. The variant N108D A102G may also be examined. Finally, because Hans-LanM and LanM_012 feature EF hands with Asn at the first position (and the position of the Gly residues in the loop is different), partial loop sequences from these proteins may also be useful to substitute into EF4.

>Mex-LanM-N108D/A124G (mutations underlined)
(SEQ ID NO: 39)
MAPTTTTKVDIAAFDPDKDGTIDLKEALAAGSAAFDKLDPDKDGT
LDAKELKGRVSEADLKKLDPDNDGTLDKKEYLAAVEAQFKAADPD
NDGTIDARELASPGGSALVNLIR
>Mex-LanM-N108D/A127G (mutations underlined)
(SEQ ID NO: 40)
MAPTTTTKVDIAAFDPDKDGTIDLKEALAAGSAAFDKLDPDKDGT
LDAKELKGRVSEADLKKLDPDNDGTLDKKEYLAAVEAQFKAADPD
NDGTIDARELASPAGSGLVNLIR
>Mex-LanM-N108D/A102G (mutations underlined)
(SEQ ID NO: 41)
MAPTTTTKVDIAAFDPDKDGTIDLKEALAAGSAAFDKLDPDKDGT
LDAKELKGRVSEADLKKLDPDNDGTLDKKEYLAAVEGQFKAADPD
NDGTIDARELASPAGSALVNLIR

Although the present disclosure has been described with respect to one or more particular embodiments and/or examples, it will be understood that other embodiments and/or examples of the present disclosure may be made without departing from the scope of the present disclosure.

1. A protein capable of binding a metal and/or metal ion, comprising a first EF hand motif, a second EF hand motif, a third EF hand motif, and a fourth EF hand motif, each EF hand motif comprising 11, 12, or 14 amino acid residues, wherein when the first EF hand motif, the second EF hand motif, the third EF hand motif, and the fourth EF hand motif have 12 amino acid residues, each EF hand motif has the following sequence: 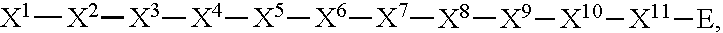
wherein,
i) for the first EF hand motif, the second EF hand motif, and the fourth EF hand motif:
each X¹ is independently D or N;
each X² is independently any canonical amino acid;
each X³ is independently D, N, or E;
each X⁴ is independently any canonical amino acid;
each X⁵ is independently D, N, or E;
each X⁶ is independently any canonical amino acid;
each X⁷ is independently any canonical amino acid;
each X⁸ is independently a hydrophobic residue;
each X⁹ is independently a D, E, or T;
each X¹⁰ is independently a hydrophobic residue; and
each X¹¹ is independently any canonical amino acid;
ii) for the third EF hand motif:
X¹ is N;
X² is any canonical amino acid;
X³ is D;
X⁴ is G or A;
X⁵ is D or N;
X⁶ is any canonical amino acid;
X⁷ is T or S;
X⁸ is a hydrophobic residue;
X⁹ is E;
X¹⁰ is a hydrophobic residue; and
X¹¹ is D; and
iii) the EF hand motifs are connected by 12 or 13 amino acid residue linkers and each amino acid residue of the linkers is a canonical amino acid, except for the third EF hand motif and the fourth EF hand motif, which are connect by the following sequence: (X)₅-R-(X)₆, wherein each X is independent a canonical amino acid and at least one amino acid of any of the linkers are hydrophobic.

2. The protein according to claim 1, wherein X⁷ of the first EF hand motif, the second EF hand motif, and/or the fourth EF hand motif is independently T or S.

3. The protein according to claim 1, wherein X⁴ of the third EF hand motif is A.

4. The protein according to claim 1, wherein X⁸ of the third EF hand motif is L.

5. The protein according to claim 1, wherein X¹⁰ of the third EF hand motif is L, I, or M.

6. The protein according to claim 1, wherein the protein comprises the sequence:
(SEQ ID NO: 1)
MKLSLKAGAA ITAFVFAASP VLAASGADAL KALNKDNDDS
LEIAEVIHAG ATTFTAINPD GDTTLESGET KGRLTEKDWA
RANKDGDQTL EMDEWLKILR TRFKRADANK DGKLTAAELD
SKAGQGVLVM IMK
or
(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK
or a protein having 70% identity to either SEQ ID NO:1 or SEQ ID NO:2.

7. The protein according to claim 1, wherein the protein comprises the following sequence:
(SEQ ID NO: 7)
MLTGKEFLRKYNKDKDSTVEIVEAIDLGTKVFKAINPDKDKTL
EAAETKGRLSDEDWAQFNKDGDKTLELDEWLIIVRKRFNDADA
NKDGKLTEAELDAPAGQQLILLIAK,
or a protein having 70% identity thereto.

8. The protein according to claim 1, wherein the protein is complexed with a rare earth element.

9. The protein according to claim 1, wherein the rare earth element is a light rare earth element.

10. The protein according to claim 8, wherein the rare earth element is a heavy rare earth element.

11. The protein according to claim 1, wherein the protein comprises the following sequence:
(SEQ ID NO: 44)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK,
where X is any canonical amino acid residue other than R.

12. The protein according to claim 11, wherein the protein comprises the following sequence:
(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK

13. A protein according to claim 1, wherein the protein has the following sequence:
>Hans-LanM
(SEQ ID NO: 1)
MKLSLKAGAA ITAFVFAASP VLAASGADAL KALNKDNDDS
LEIAEVIHAG ATTFTAINPD GDTTLESGET KGRLTEKDWA
RANKDGDQTL EMDEWLKILR TRFKRADANK DGKLTAAELD
SKAGQGVLVM IMK;
>Hans-LanM
(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK;
>Hans-LanM(R100K)
(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK;
>Hans-LanM-Cys
(SEQ ID NO: 5)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMKGSGC;
>Hans-LanM(R100K)-Cys
(SEQ ID NO: 6)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMKGSGC;
>LanM_012
(SEQ ID NO: 7)
MLTGKEFLRKYNKDKDSTVEIVEAIDLGTKVFKAINPDKDKTLEA
AETKGRLSDEDWAQFNKDGDKTLELDEWLIIVRKRFNDADANKDG
KLTEAELDAPAGQQLILLIAK;
>LanM_011
(SEQ ID NO: 14)
MGHHNCKAEMAYLNPDHDGTIDWREARRAAVRLFHKLDPDHDGTL
DMKEVRGRVGILSFARFNPDRDGKLDKHEWLALVKHRFHRANPDK
DGTIDCRELHSLAGRKLLRVLM;
>HansR100K-L1
(SEQ ID NO: 16)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSASGADA
LKALNKDNDDSLEIAEVIHAGATTFTAINPDGDTTLESGETKGRL
TEKDWARANKDGDQTLEMDEWLKILKTRFKRADANKDGKLTAAEL
DSKAGQGVLVMIMK;
>HansR100K-L2
(SEQ ID NO: 17)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSASGADALKALNKDNDDSLEIAEVIHAGATTFTAINPDGD
TTLESGETKGRLTEKDWARANKDGDQTLEMDEWLKILKTRFKRAD
ANKDGKLTAAELDSKAGQGVLVMIMK;
>HansR100K-L3
(SEQ ID NO: 18)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSGGSGGSASGADALKALNKDNDDSLEIAEVIHAGATTFTA
INPDGDTTLESGETKGRLTEKDWARANKDGDQTLEMDEWLKILKT
RFKRADANKDGKLTAAELDSKAGQGVLVMIMK;
>HansR100K-L4
(SEQ ID NO: 19)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSGGSGGSGGSGGSASGADALKALNKDNDDSLEIAEVIHAG
ATTFTAINPDGDTTLESGETKGRLTEKDWARANKDGDQTLEMDEW
LKILKTRFKRADANKDGKLTAAELDSKAGQGVLVMIMK;
>HansR100K-L5
(SEQ ID NO: 20)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGSGGSGAEAAAKEAAAKAGGSGGS
AEAAAKEAAAKAGSGGSGASGADALKALNKDNDDSLEIAEVIHAG
ATTFTAINPDGDTTLESGETKGRLTEKDWARANKDGDQTLEMDEW
LKILKTRFKRADANKDGKLTAAELDSKAGQGVLVMIMK;
>Hans-LanM-I43A
(SEQ ID NO: 26)
MASGADALKALNKDNDDSLEAAEVIHAGATTFTAINPDGDTTLES
GETKGRLTEKDWARANKDGDQTLEMDEWLKILRTRFKRADANKDG
KLTAAELDSKAGQGVLVMIMK;
>Hans-LanM-I43V
(SEQ ID NO: 27)
MASGADALKALNKDNDDSLEVAEVIHAGATTFTAINPDGDTTLES
GETKGRLTEKDWARANKDGDQTLEMDEWLKILRTRFKRADANKDG
KLTAAELDSKAGQGVLVMIMK;
>Hans-LanM-A44N
(SEQ ID NO: 28)
MASGADALKALNKDNDDSLEINEVIHAGATTFTAINPDGDTTLES
GETKGRLTEKDWARANKDGDQTLEMDEWLKILRTRFKRADANKDG
KLTAAELDSKAGQGVLVMIMK;
>Hans-LanM-A44S
(SEQ ID NO: 29)
MASGADALKALNKDNDDSLEISEVIHAGATTFTAINPDGDTTLES
GETKGRLTEKDWARANKDGDQTLEMDEWLKILRTRFKRADANKDG
KLTAAELDSKAGQGVLVMIMK;
>Hans-LanM-A44T
(SEQ ID NO: 30)
MASGADALKALNKDNDDSLEITEVIHAGATTFTAINPDGDTTLES
GETKGRLTEKDWARANKDGDQTLEMDEWLKILRTRFKRADANKDG
KLTAAELDSKAGQGVLVMIMK;
>Hans-LanM-I47A
(SEQ ID NO: 31)
MASGADALKALNKDNDDSLEIAEVAHAGATTFTAINPDGDTTLES
GETKGRLTEKDWARANKDGDQTLEMDEWLKILRTRFKRADANKDG
KLTAAELDSKAGQGVLVMIMK;
>Hans-LanM-147V
(SEQ ID NO: 32)
MASGADALKALNKDNDDSLEIAEVVHAGATTFTAINPDGDTTLES
GETKGRLTEKDWARANKDGDQTLEMDEWLKILRTRFKRADANKDG
KLTAAELDSKAGQGVLVMIMK;
>Hans-LanM-M92L
(SEQ ID NO: 33)
MASGADALKALNKDNDDSLEIAEVIHAGATTFTAINPDGDTTLES
GETKGRLTEKDWARANKDGDQTLELDEWLKILRTRFKRADANKDG
KLTAAELDSKAGQGVLVMIMK;
>Hans-LanM-M92A
(SEQ ID NO: 34)
MASGADALKALNKDNDDSLEIAEVIHAGATTFTAINPDGDTTLES
GETKGRLTEKDWARANKDGDQTLEADEWLKILRTRFKRADANKDG
KLTAAELDSKAGQGVLVMIMK;
>Hans-LanM-M92D
(SEQ ID NO: 35)
MASGADALKALNKDNDDSLEIAEVIHAGATTFTAINPDGDTTLES
GETKGRLTEKDWARANKDGDQTLEDDEWLKILRTRFKRADANKDG
KLTAAELDSKAGQGVLVMIMK;
>Hans-LanM-D93A
(SEQ ID NO: 36)
MASGADALKALNKDNDDSLEIAEVIHAGATTFTAINPDGDTTLES
GETKGRLTEKDWARANKDGDQTLEMAEWLKILRTRFKRADANKDG
KLTAAELDSKAGQGVLVMIMK;
>Hans-LanM-D93N
(SEQ ID NO: 37)
MASGADALKALNKDNDDSLEIAEVIHAGATTFTAINPDGDTTLES
GETKGRLTEKDWARANKDGDQTLEMNEWLKILRTRFKRADANKDG
KLTAAELDSKAGQGVLVMIMK;
>HansR100K-L1
(SEQ ID NO: 16)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSASGADA
LKALNKDNDDSLEIAEVIHAGATTFTAINPDGDTTLESGETKGRL
TEKDWARANKDGDQTLEMDEWLKILKTRFKRADANKDGKLTAAEL
DSKAGQGVLVMIMK;
>HansR100K-L2
(SEQ ID NO: 17)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSASGADALKALNKDNDDSLEIAEVIHAGATTFTAINPDGD
TTLESGETKGRLTEKDWARANKDGDQTLEMDEWLKILKTRFKRAD
ANKDGKLTAAELDSKAGQGVLVMIMK;
>HansR100K-L3
(SEQ ID NO: 18)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSGGSGGSASGADALKALNKDNDDSLEIAEVIHAGATTFTA
INPDGDTTLESGETKGRLTEKDWARANKDGDQTLEMDEWLKILKT
RFKRADANKDGKLTAAELDSKAGQGVLVMIMK;
>HansR100K-L4
(SEQ ID NO: 19)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGGSGGSGGSGGSGGSGGSGGSGGS
GGSGGSGGSGGSGGSGGSASGADALKALNKDNDDSLEIAEVIHAG
ATTFTAINPDGDTTLESGETKGRLTEKDWARANKDGDQTLEMDEW
LKILKTRFKRADANKDGKLTAAELDSKAGQGVLVMIMK;
>HansR100K-L5
(SEQ ID NO: 20)
MASGADALKALNKDNDDSLEIAEVIHAGATTFTAINPDGDTTLES
GETKGRLTEKDWARANKDGDQTLEMDEWLKILKTRFKRADANKDG
KLTAAELDSKAGQGVLVMIMKGSGGSGAEAAAKEAAAKAGGSGGS
AEAAAKEAAAKAGSGGSGASGADALKALNKDNDDSLEIAEVIHAG
ATTFTAINPDGDTTLESGETKGRLTEKDWARANKDGDQTLEMDEW
LKILKTRFKRADANKDGKLTAAELDSKAGQGVLVMIMK;
or
>Hans-LanM(3E9Q)
(SEQ ID NO: 38)
MASGADAL KALNKDNDDS LQIAEVIHAG ATTFTAINPD
GDTTLQSGET KGRLTEKDWA RANKDGDQTL QMDEWLKILR
TRFKRADANK DGKLTAAELD SKAGQGVLVM IMK.

14. A protein according to claim 1, wherein the protein has the following sequence:
(SEQ ID NO: 1)
MKLSLKAGAA ITAFVFAASP VLAASGADAL KALNKDNDDS
LEIAEVIHAG ATTFTAINPD GDTTLESGET KGRLTEKDWA
RANKDGDQTL EMDEWLKILR TRFKRADANK DGKLTAAELD
SKAGQGVLVM IMK;
(SEQ ID NO: 2)
MASGADAL KALNKDNDDS LEIAEVIHAG ATTFTAINPD
GDTTLESGET KGRLTEKDWA RANKDGDQTL EMDEWLKILR
TRFKRADANK DGKLTAAELD SKAGQGVLVM IMK;
or
(SEQ ID NO: 4)
MASGADAL KALNKDNDDS LEIAEVIHAG ATTFTAINPD
GDTTLESGET KGRLTEKDWA RANKDGDQTL EMDEWLKILK
TRFKRADANK DGKLTAAELD SKAGQGVLVM IMK.

15. A protein having an enhanced REE/REE selectivity, comprising a first EF hand motif, a second EF hand motif, a third EF hand motif, and a fourth EF hand motif, each EF hand motif comprising 11, 12, or 14 amino acid residues, wherein when the first EF hand motif, the second EF hand motif, the third EF hand motif, and the fourth EF hand motif have 12 amino acid residues, each EF hand motif has the following sequence: 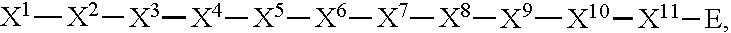
wherein,
i) for the first EF hand motif and the fourth EF hand motif:
each X¹ is independently D or N;
each X² is independently any canonical amino acid;
each X³ is independently D, N, or E;
each X⁴ is independently any canonical amino acid;
each X⁵ is independently D, N, or E;
each X⁶ is independently any canonical amino acid;
each X⁷ is independently any canonical amino acid;
each X⁸ is independently a hydrophobic residue;
each X⁹ is independently a D, E, or T;
each X¹⁰ is independently a hydrophobic residue; and
each X¹¹ is independently any canonical amino acid;
ii) for the second EF hand motif:
X¹ is N;
X² is any canonical amino acid;
X³ is D;
X⁴ is any canonical amino acid;
X⁵ is D;
X⁶ is any canonical amino acid;
X⁷ is T or S;
X⁸ is a hydrophobic residue;
X⁹ is E;
X¹⁰ is any canonical amino acid; and
X¹¹ is any canonical amino acid; and
iii) for the third EF hand motif:
X¹ is D;
X² is any canonical amino acid;
X³ is D;
X⁴ is D;
X⁵ is D;
X⁶ is G;
X⁷ is T or S;
X⁸ is a hydrophobic residue;
X⁹ is D;
X¹⁰ is any canonical amino acid; and
X¹¹ is any canonical amino acid;
iv) at least one X² of the second EF hand motif and third EF hand motif is P; and
v) the EF hand motifs are connected by 12 or 13 amino acid residue linkers, wherein each amino acid of the linkers is a canonical amino acid and at least one amino acid of any of the linkers are hydrophobic.

16. The protein of claim 15, wherein the protein has the following sequence:
>Mex-LanM-G51A
(SEQ ID NO: 21)
MAPTTTTKVDIAAFDPDKDGTIDLKEALAAASAAFDKLDPDKDGT
LDAKELKGRVSEADLKKLDPDNDGTLDKKEYLAAVEAQFKAANPD
NDGTIDARELASPAGSALVNLIR;
>Mex-LanM-A98G
(SEQ ID NO: 22)
MAPTTTTKVDIAAFDPDKDGTIDLKEALAAGSAAFDKLDPDKDGT
LDAKELKGRVSEADLKKLDPDNDGTLDKKEYLGAVEAQFKAANPD
NDGTIDARELASPAGSALVNLIR;
>Mex-LanM-A99G
(SEQ ID NO: 23)
MAPTTTTKVDIAAFDPDKDGTIDLKEALAAGSAAFDKLDPDKDGT
LDAKELKGRVSEADLKKLDPDNDGTLDKKEYLAGVEAQFKAANPD
NDGTIDARELASPAGSALVNLIR;
>Mex-LanM-V100G
(SEQ ID NO: 24)
MAPTTTTKVDIAAFDPDKDGTIDLKEALAAGSAAFDKLDPDKDGT
LDAKELKGRVSEADLKKLDPDNDGTLDKKEYLAAGEAQFKAANPD
NDGTIDARELASPAGSALVNLIR;
>Mex-LanM-A102G
(SEQ ID NO: 25)
MAPTTTTKVDIAAFDPDKDGTIDLKEALAAGSAAFDKLDPDKDGT
LDAKELKGRVSEADLKKLDPDNDGTLDKKEYLAAVEGQFKAANPD
NDGTIDARELASPAGSALVNLIR;
>LanM_013
(SEQ ID NO: 8)
MGKAADAIQALDPDKDGTIDLNEAKAGAKAVFEKINPDGDGTLEV
KELKGRLTKKELDAADPDNDGTLDMQEYEAVVTKQFELANPDNDG
TVDEKELKTKEGKKLLKLIY;
>Mex-LanM-A32D/A117R
(SEQ ID NO: 12)
MAPTTTTKVDIDAFDPDKDGTIDLKEALAAGSAAFDKLDPDKDGT
LDAKELKGRVSEADLKKLDPDNDGTLDKKEYLAAVEAQFKAANPD
NDGTIDRRELASPAGSALVNLIR;
>LanM_013:
(SEQ ID NO: 9)
MAAILTIAGAVTVAAGGAAFAGKAADAIQALDPDKDGTIDLNEAK
AGAKAVFEKINPDGDGTLEVKELKGRLTKKELDAADPDNDGTLDM
QEYEAVVTKQFELANPDNDGTVDEKELKTKEGKKLLKLIY;
>Mex-LanM-A32D/A117K
(SEQ ID NO: 11)
MAPTTTTKVDIDAFDPDKDGTIDLKEALAAGSAAFDKLDPDKDGT
LDAKELKGRVSEADLKKLDPDNDGTLDKKEYLAAVEAQFKAANPD
NDGTIDKRELASPAGSALVNLIR;
>Mex-LanM-I42L/N108D/1115L
(SEQ ID NO: 13)
MAPTTTTKVDIAAFDPDKDGTLDLKEALAAGSAAFDKLDPDKDGT
LDAKELKGRVSEADLKKLDPDNDGTLDKKEYLAAVEAQFKAADPD
NDGTLDARELASPAGSALVNLIR;
>Mex-LanM-N108D/A124G
(SEQ ID NO: 39)
MAPTTTTKVDIAAFDPDKDGTIDLKEALAAGSAAFDKLDPDKDGT
LDAKELKGRVSEADLKKLDPDNDGTLDKKEYLAAVEAQFKAADPD
NDGTIDARELASPGGSALVNLIR;
>Mex-LanM-N108D/A124G
(SEQ ID NO: 40)
MAPTTTTKVDIAAFDPDKDGTIDLKEALAAGSAAFDKLDPDKDGT
LDAKELKGRVSEADLKKLDPDNDGTLDKKEYLAAVEAQFKAADPD
NDGTIDARELASPAGSGLVNLIR;
>Mex-LanM-N108D/A102G
(SEQ ID NO: 41)
MAPTTTTKVDIAAFDPDKDGTIDLKEALAAGSAAFDKLDPDKDGT
LDAKELKGRVSEADLKKLDPDNDGTLDKKEYLAAVEGQFKAADPD
NDGTIDARELASPAGSAL VNLIR;
>Unclassified Hyphomicrobium
(SEQ ID NO: 15)
MGHRSAKAHPSCPALNAIDPDGDGAMTLGEAKRAAIKTFMKLNKD
GDITLELDELGGRMSAAAFAQADLIKGRGISLGEYLIEVRRRFKW
ANPDKDHTIECDELHSKYGRLLARLLK;
>Mex-LanM-N108D
(SEQ ID NO: 42)
MAPTTTTKVDIAAFDPDKDGTIDLKEALAAGSAAFDKLDPDKDGT
LDAKELKGRVSEADLKKLDPDNDGTLDKKEYLAAVEAQFKAADPD
NDGTIDARELASPAGSALVNLIR;
or
>Methyloligella halotolerans
(SEQ ID NO: 10)
MADAEISDTMKVVDPDMDNALTLEEAQAAGAKVFKKLNTDDDNTL
EADELKGRVSERQLKKADPDDDGSLDMAEYEALIKKRFEAANPDG
DDTIESDELETKKGKKLLELIQE.

17. The protein according to claim 15, wherein X⁸ of the second EF hand motif and/or third EF hand motif is L, I, M, or V.

18. A device comprising a protein according to claim 1 or claim 15.

19. The device according to claim 18, wherein the device is a filter, membrane, sensor, handheld detector, plate reader, fluorimeter, biosensor, or in-line monitor.

20. A kit comprising a protein according to claim 1 or claim 15 or a device comprising a protein according to claim 1 or claim 15.

21. A method of isolating a rare earth element comprising contacting a protein of claim 1 with a sample comprising a rare earth element, wherein the rare earth element binds to one or more proteins of claim 1, and removing the protein from the sample.

22. The method according to claim 21, wherein the sample is drinking water, wastewater, ground water, ash ponds, aqueous extract from contaminated soil, drainage, leachate, aqueous extract or leachate from a solid waste such as electronic waste or from an ore or mine tailings, or solid sample.

23. The method according to claim 21, wherein the one or more rare earth element is a lanthanide are chosen from La, Ce, Pr, Nd, Pm, Sm, Eu, Gd, Tb, Dy, Ho, Er, Tm, Yb, Lu, Sc, Y, and ions thereof.

24. The method according to claim 21, wherein the method further comprises detecting and quantifying the one or more rare earth element.

25. The method according to claim 21, wherein a plurality of different rare earth elements are bound to the protein.

26. The method according to claim 25, wherein each different rare earth element is separated individually from the protein.

27. A method of determining if a light rare earth element is present in a sample, comprising contacting the sample with a protein according to claim 1, and determining if the protein has formed a dimer.
